(19) United States
(12) Patent Application Publication
(10) Pub. No.: US 2004/0254162 A1
(43) Pub. Date: Dec. 16, 2004

(54) Oxazolidinone derivatives as antimicrobials

(76) Inventors: Anita Mehta, Buffalo Grove, IL (US); Sudershan R Arora, Gurgaon (IN); Bisjawit Das, New Delhi (IN); Abhijit Ray, Elkridge, MD (US); Sonali Rudra, New Delhi (IN); Ashok Rattan, New Delhi (IN)

Correspondence Address: RANBAXY INC., 600 COLLEGE ROAD EAST, SUITE 2100, PRINCETON, NJ, 08540, US

(21) Appl. No.: 10/483,904

(22) PCT Filed: Oct. 5, 2002
(86) PCT No.: PCT/IB02/01609

(30) Foreign Application Priority Data
Jul. 16, 2001 (WO) PCT/IB01/01262

(51) Int. Cl. A61K 31/55; A61K 31/496; A61K 31/454; C07D 413/14

(52) U.S. Cl. 514/217.05; 514/253.09; 514/254.02; 514/326; 540/598; 544/360; 544/369; 546/187; 546/209

(57) ABSTRACT

The present invention relates to certain substituted phenyl oxazolidinones and to processes for the synthesis of the same. This invention also relates to pharma-ceutical compositions containing the compounds of the present invention as anti-microbials. The compounds are useful antimicrobial agents, effective against a number of human and veterinary pathogens, including gram-positive aerobic bacteria such as multiply-resistant staphylococci, streptococci and enterococci as well as anaerobic organisms such as Bacterioides spp. and Clostridia spp. species, and acid fast organisms such as Mycobacterium tuberculosis, Mycobacterium avium and Mycobacterium spp.

### FIELD OF THE INVENTION

The present invention relates to certain substituted phenyl oxazolidinones and to processes for the synthesis of the same. This invention also relates to pharmaceutical compositions containing the compounds of the present invention as antimicrobials. The compounds are useful antimicrobial agents, effective against a number of human and veterinary pathogens, including gram-positive aerobic bacteria such as multiply-resistant staphylococci, streptococci and enterococci as well as anaerobic organisms such as Bacterioides spp. and Clostridia spp. species, and acid fast organisms such as Mycobacterium tuberculosis, Mycobacterium avium and Mycobacterium spp.

### BACKGROUND OF THE INVENTION

Increasing antibacterial resistance in Gram positive bacteria has presented a formidable treatment problem. The enterococci, although traditionally non virulent pathogens, have been shown, when associated with Vancomycin resistance, to have an attributable mortality of approximately 40%. Staphylococcus aureus, the traditional pathogen of post operative wounds, has been resistant to Penicillin due to production of penicillinases. This resistance was overcome by the development of various penicillinase stable β lactams. But the pathogen responded by synthesizing a modified target penicillin binding protein-2′ leading to less affinity for β lactam antibiotics and a phenotype known as Methicillin Resistant S. aureus (MRSA). These strains, till recently were susceptible to Vancomycin, which inspite of its various drawbacks, has become the drug of choice for MRSA infections. Streptococcus pneumoniae is a major pathogen causing pneumonia, sinusitis and meningitis. Until very recently it was highly susceptible to penicillin. Recently though, different PBP 2′ strains with different susceptibility to penicillin have been reported from across the globe.

Oxazolidinones are a new class of synthetic antimicrobial agents which kill gram positive pathogens by inhibiting a very early stage of protein synthesis. Oxazolidinones inhibit the formation of ribosomal initiation complex involving 30S and 50S ribosomes leading to prevention of initiation complex formation. Due to their novel mechanism of action, these compounds are active against pathogens resistant to other clinically useful antibiotics.

WO93/23384 application discloses phenyloxazolidinones containing a substituted diazine moiety and their uses as antimicrobials.

WO93/09103 application discloses substituted aryl and heteroaryl-phenyl-oxazolidinones useful as antibacterial agents

WO90/02744 application discloses 5-indolinyl-5β-amidomethyloxazolidinones, 3-(fused ring substituted) phenyl-5β-amidomethyloxazolidinones which are useful as antibacterial agents.

European Patent Publication 352,781 discloses phenyl and pyridyl substituted phenyl oxazolidinones.

European Patent Application 312,000 discloses phenylmethyl and pyridinylmethyl substituted phenyl oxazolidinones.

U.S. Pat. No. 5,254,577 discloses nitrogen heteroaromatic rings attached to phenyloxazolidinone.

U.S. Pat. Nos. 5,547,950 and 5,700,799 also disclose the phenyl piperazinyl oxazolidinones.

Other references disclosing various phenyloxazolidinones include U.S. Pat. Nos. 4,801,600 and 4,921,869; Gregory W. A., et al., J.Med.Chem., 32, 1673-81 (1989); Gregory W. A., et al., J.Med.Chem., 33, 2569-78 (1990); Wang C., et al., Tetrahedron, 45, 1323-26 (1989); Brittelli, et al., J.Med. Chem., 35, 1156 (1992); and Bio-organic and Medicinal Chemistry Letters, 9, pp. 2679-2684, 1999.

### SUMMARY OF THE INVENTION

The objective of this invention is to synthesize, identify and profile oxazolidinone molecules which have good activity against multiply resistant gram positive pathogens like MRSA, VRE and PRSP. Some of these molecules have activity against MDR-TB and MAI strains, while others have significant activity against important anaerobic bacteria.

The compounds of the present invention are related by their substituted phenyloxazolidinone ring structure in the compounds disclosed to the publications described above except that the subject compounds have a diazine moiety attached to the phenyloxazolidinone which is further substituted by heterocyclic, aryl, substituted aryl, heteroaroamatic ring therefore the compounds are unique and have superior antibacterial activity.

Another object of the present invention is to provide processes for the novel phenyloxazolidinones derivatives that exhibit significantly greater antibacterial activity, than available with the present compounds against multiply resistant gram positive pathogens like MRSA, VRE and PRSP against MDR-TB and MAI strains, in order to provide safe and effective treatment of bacterial infections.

In order to achieve the above-mentioned objectives and in accordance with the purpose of the invention as embodied and broadly described herein, there is provided a process for the synthesis of novel phenyloxazolidinone derivatives represented by Formula I

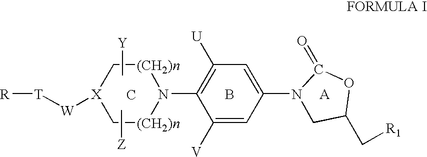

wherein

T is five to seven membered heterocyclic ring, aryl, substituted aryl, bound to the ring C with a linker W, preferred forms of T are selected from aryl and five membered heteroaryl which are further substituted by a group represented by R, wherein R is selected from the group consisting of alkyl (C₁-C₆), halogen, —CN, COR₅, COOR₅, N(R₆, R₇), CON(R₆, R₇), CH₂NO₂, NO₂, CH₂R₈, CHR₉, —CH═N—OR₁₀, —C═CH—R₅, wherein R₅ is selected from H, optionally substituted C₁-C₁₂, alkyl, C₃-₁₂, cycloalkyl, aryl, heteroaryl, R₆ and R₇, are independently selected from H, optionally substituted C₁-₁₂ alkyl, C₃-₁₂ cycloalkyl, C₁-₆ alkoxy; R₈ and R₉ are independently selected from H, C₁-₆ alkyl, F, Cl, Br, C₁-₁₂ alkyl substituted with one or more of F, Cl, Br, I, OR₄, SR₄, N(R₆, R₇) wherein R₄ is selected from H, C₁₋₁₂ alkyl, C₃₋₁₂ cycloalkyl, C₁₋₆ alkoxy, C₁₋₆ alkyl substituted with one or more F, Cl, Br, I or OH and R₆ and R₇ are the same as defined earlier, Rio is selected from H, optionally substituted C₁₋₁₂ alkyl, C₃₋₁₂ cycloalkyl, C₁₋₆ alkoxy, C₁₋₆ alkyl, aryl, heteroaryl; n is an integer in the range from 0 to 3;

X is CH, CH—S, CH—O and N;

Y and Z are independently selected from hydrogen, C₁₋₆ alkyl, C₃₋₁₂ cycloalkyl, C₀₋₃ bridging groups;

U and V are independently selected from optionally substituted C₁₋₆ alkyl, F, Cl, Br, C ₁₋₁₂ alkyl substituted with one or more of F, Cl, Br, I, preferably U and V are hydrogen or fluoro;

W is selected from the group CH₂, CO, CH₂NH, —NHCH₂, —CH₂NHCH₂, —CH₂—N(R₁₁)CH₂—, —CO—CO—, CH₂(R₁₁)N—, CH(R₁₁), S, CH₂(CO), N(R₁₁) wherein R₁₁ is hydrogen, optionally substituted C₁₋₁₂ alkyl, C₃₋₁₂ cycloalkyl, C₁₋₆ alkoxy, C₁₋₆alkyl, aryl, or heteroaryl;

R₁ is selected from the group consisting of —NHC(═O)R₂ wherein R₂ is hydrogen, C₁₋₁₂alkyl, C₃₋₁₂ cycloalkyl, C₁₋₆ alkoxy, C₁₋₆ alkyl substituted with one or more of F, Cl, Br, I or OH, N(R₃, R₄), —NR₂C(═S)R₃, —NR₂C(═S)SR₃ wherein R₂ is the same as defined above, R₃, R₄ are independently selected from hydrogen, C₁₋₁₂ alkyl, C₃₋₁₂ cycloalkyl, C₁₋₆ alkoxy, C₁₋₆ alkyl substituted with one or more of F, Cl, Br, I or OH.

Preferred compounds of Formula I have R₁ as acetamide and the most preferred compounds in this series would be prepared as the optically pure enantiomers having the (S)-configuration according to the Cahn-Ingold-Prelog notation at C₅ of the oxazolidinone ring. The (S)-enantiomer of this series of compounds is preferred since it has two times more antibacterial activity than the corresponding racemic compound. The scope of the individual isomers and mixture of enantiomers of the structural Formula I are also covered in this invention.

Still more preferred compounds of the Formula I containing D ring as furanyl, thienyl and pyrrolyl ring systems (M═O,S, NH, N—CH₃) and further substituted by substitutions Q and P is represented by Formula II

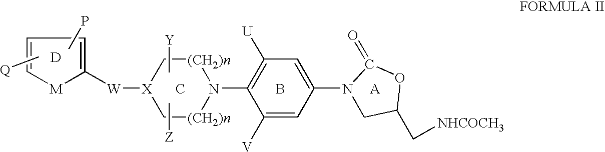

wherein

U and V are independently selected from optionally substituted C₁₋₆ alkyl, F, Cl, Br, Cl₁₋₁₂ alkyl substituted with one or more of F, Cl, Br, I, preferably U and V are hydrogen or fluoro;

X is CH, CH—S, CH—O and N;

Y and Z are independently selected from hydrogen, C₁₋₆ alkyl, C₃₋₁₂ cycloalkyl, C₀₋₃ bridging groups;

n is an integer in the range from 0 to 3;

W is selected from the group CH₂, CO, CH₂NH, —NHCH₂, —CH₂NHCH₂, —CH₂—N(R₁₁)CH₂—, —CO—CO—, CH₂(R₁₁)N—, CH(R₁₁), S, CH₂(CO), N(R₁₁) wherein

R₁₁ is hydrogen, optionally substituted C₁₋₁₂alkyl, C₃₋₁₂cycloalkyl, C₁₋₆ alkoxy, C₁₋₆ alkyl, aryl, heteroaryl.

Preferred compounds of Formula II of this invention are those when Q and P are independently selected from the group consisting of hydrogen, —CN, COR₅, COOR₅, N(R₆, R₇), CON(R₆,R₇), CH₂NO₂, N₂, CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, or heteroaryl; R₆, R₇ are independently selected from H, optionally substituted C ₁₋₁₂ alkyl, C₃₋₁₂ cycloalkyl, C₁₋₆ alkoxy, R₈, R₉ and are independently selected from the group consisting of H, C₁₋₆ alkyl, F, Cl, Br, C₁₋₁₂ alkyl substituted with one or more of F, Cl, Br, I, OR₄, SR₄, wherein R₄ is the same as defined earlier, N(R₆, R₇), R₁₀═H, optionally substituted C ₁₋₁₂ alkyl, C₃₋₁₂ cycloalkyl, C₁₋₆ alkoxy, C₁₋₆ alkyl, aryl, heteroaryl except when W=(CO), Q and P=H and M=S.

In the more preferred compounds represented by Formula II ring C may be 6-8 membered in size and the larger rings may have either two or three carbons between each nitrogen atom, for example:

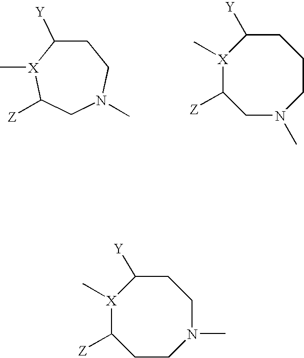

The ring C may be bridged to form a bicyclic system as shown below:

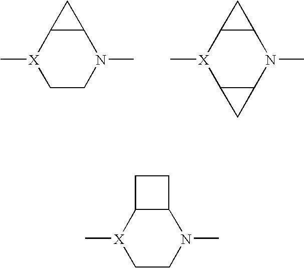

When ring C is optionally substituted at positions Y and Z with alkyl groups, cycloalkyl groups, fluoro group, carboxylic and corresponding esters, amides, substituted alkyls or bridging alkyl groups are as shown below:

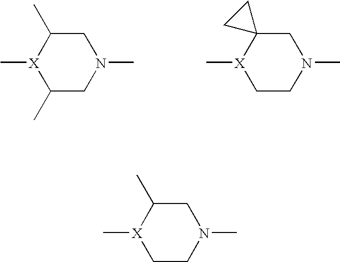

When ring C is 6 membered in size and X is —CH—N(R₁₁), the following rings are preferred ones wherein R₁₁ is the same as defined earlier.

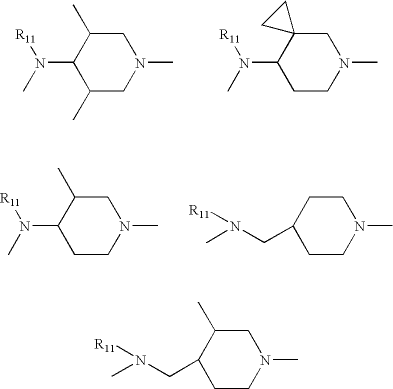

In addition to the above, ring C also includes the following structures:

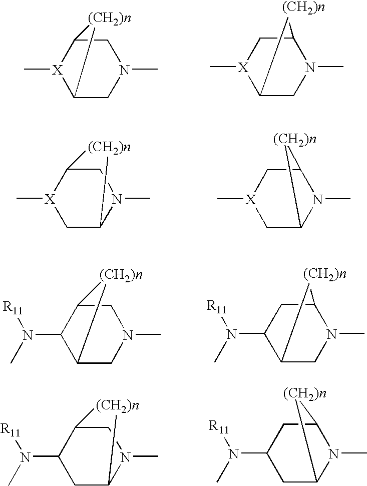

Still more preferred compounds of Formula II when M=Sulphur is represented by Formula III

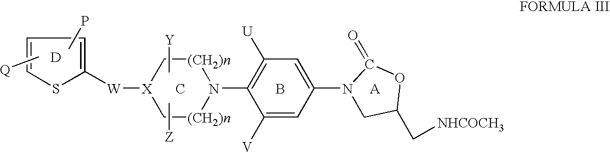

wherein

U and V are independently selected from optionally substituted C₁₋₆ alkyl, F, Cl, Br, C₁₋₁₂ alkyl substituted with one or more of F, Cl, Br, I, preferably U and V are hydrogen or fluoro;

X is CH, CH—S, CH—O and N;

Y and Z are independently selected from hydrogen, C₁₋₆ alkyl, C₃₋₁₂ cycloalkyl and C₀₋₃ bridging groups;

W is selected from the group CH₂, CO, CH₂NH, —NHCH₂, —CH₂NHCH₂, —CH₂—N(R₁₁)CH₂—, —CO—CO—, CH₂(R₁₁)N—, CH(R₁₁), S, CH₂(CO), N(R₁₁) wherein R₁₁ is hydrogen, optionally substituted C₁₋₁₂alkyl, C₃₋₁₂ cycloalkyl, C₁₋₆ alkoxy, C₁₋₆ alkyl, aryl, heteroaryl; and,

Q and P are independently selected from the group consisting of hydrogen, —CN, COR₅, COOR₅, N(R₆, R₇), CON(R₆, R₇), CH₂NO₂, NO₂, CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, or heteroaryl; R₆, R₇ are independently selected from H, optionally substituted C₁₋₁₂ alkyl, C₃₋₁₂ cycloalkyl, C₁₋₆ alkoxy, R₈, R₉ and are independently selected from the group consisting of H, C₁₋₆ alkyl, F, Cl, Br, C₁₋₁₂ alkyl substituted with one or more of F, Cl, Br, I, OR₄, SR₄, wherein R₄ is the same as defined earlier, N(R₆, R₇), R₁₀═H, optionally substituted C₁₋₁₂ alkyl, C₃₋₁₂ cycloalkyl, C₁₋₆ alkoxy, C₁₋₆ alkyl, aryl, heteroaryl except W=(CO), Q and P=H.

More preferred Q, P substitutions are nitro, aldehydes and halides.

Preferably W is selected from the groups consisting of CH₂, C(═O), C(═O)—C(═O), CH₂NH, —NHCH₂, —CH₂NHCH₂, —CH₂—N(CH₃)CH₂—, CH₂(CH₃)N—, CH(CH₃), S and CH₂(C═O), —NH. The most preferred compounds of Formula III are as follows:

-(S)-N-[[3-[4-[4-(N-methyl-N-2-thienyl(5-nitro)methyl)aminopiperidine-1-yl]-3-fluorophenyl]-2-oxo-oxazolidin-5-yl]methyl]acetamide

-(S)-N[[3-[3-Fluoro-4-[N-1[4-{2-(2-thienyl)dicarbonyl}]piperazinyl]phenyl]2-oxo-5-oxazolidinyl]methyl]acetamide

-(S)-N[[3-[3-Fluoro-4-[N-1[4-(5-nitro-2-thienoyl)]piperazinyl]phenyl]2-oxo-5-oxazolidinyl]methyl]acetamide hydrochloride

Still more preferred compounds of Formula II is represented by Formula IV (M=O)

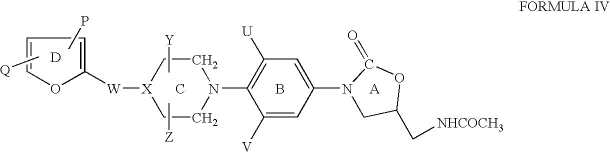

containing oxygen atom in ring D of Formula II, wherein

U and V are independently selected from optionally substituted C₁₋₆ alkyl, F, Cl, Br, C₁₋₁₂ alkyl substituted with one or more of F, Cl, Br, I, preferably U and V are hydrogen or fluoro;

X is CH, CH—S, CH—O and N;

Y and Z are independently selected from hydrogen, C₁₋₆ alkyl, C₃₋₁₂ and cycloalkyl C₀₋₃ bridging groups;

W is selected from the group CH₂, CO, CH₂NH, —NHCH₂, —CH₂NHCH₂, —CH₂—N(R₁₁)CH₂—, —CO—CO—, CH₂(R₁₁)N—, CH(R₁₁), S, CH₂(CO), N(R₁₁) wherein R₁₁ is hydrogen, optionally substituted C₁₋₁₂ alkyl, C₃₋₁₂ cycloalkyl, C₁₋₆ alkoxy, C₁₋₆ alkyl, aryl, heteroaryl; and,

Q and P are independently selected from the group consisting of hydrogen, —CN, COR₅, COOR₅, N(R₆, R₇), CON(R₆, R₇), CH₂NO₂, NO₂, CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, or heteroaryl; R₆, R₇ are independently selected from H, optionally substituted C₁₋₁₂ alkyl, C₃₋₁₂ cycloalkyl, C₁₋₆ alkoxy, R₈, R₉and are independently selected from the group consisting of H, C₁₋₆ alkyl, F, Cl, Br, C₁₋₁₂ alkyl substituted with one or more of F, Cl, Br, I, OR₄, SR₄, N(R₆, R₇), R₁₀═H, optionally substituted C ₁₋₁₂ alkyl, C₃₋₁₂ cycloalkyl, C₁₋₆ alkoxy, C₁₋₆ alkyl, aryl, heteroaryl except W=(CO), Q and P=H, M=S.

More preferred Q and P substitutions are nitro, aldehydes and halides.

Preferably W is selected from the groups consisting of CH₂, C(═O), C(═O)—C(═O), CH₂NH, —NHCH, —CH₂NHCH₂, —CH₂—N(CH₃)CH₂—, CH₂(CH₃)N—, CH(CH₃), S, CH₂(C═O), and —NH.

The most preferred compounds of Formula IV are as follows:

-(S)-N-[[3-Fluoro-4-[N-1[4-(5-nitro-2-furoyl)piperazinyl]phenyl]-2-oxo-5-oxazolidinyl]methyl]acetamide.

-(S)-N-[[3-[3-fluoro-4-[N-1-[4-{2-furyl-(5-nitro)methyl}]piperazinyl]phenyl]-2-oxo-5-oxazolidinyl]methyl]acetamide.

-(S)-N-[[3-[4-[4-(N-methyl-N-(5-nitro-2-furoyl)aminopiperidine-1-yl]-3-fluorophenyl]-2-oxo-oxazolidin-5-yl]methyl]acetamide.

-(S)-N-[[3-[4-[4-(N-methyl-N-2-furyl(5-nitro)methyl)aminopiperidine-1-yl]-3-fluorophenyl]-2-oxo-oxazolidin-5-yl]methyl]acetamide.

The compounds of the present invention are useful as antimicrobial agents, effective against a number of human and veterinary pathogens, particularly aerobic Gram-positive bacteria, including multiply-antibiotic resistant staphylococci and streptococci, as well as anaerobic organisms and Mycobacterium tuberculosis and other mycobacterium species.

For preparing pharmaceutical compositions from the compounds described by this invention, inert, pharmaceutically acceptable carriers can be either solid or liquid. Solid form preparations include powders, tablets, dispersible granules, capsules, cachets, suppositories, and ointments. A solid carrier can be one or more substances which may also act as diluents, flavouring agents, solubilizers, lubricants, suspending agents, binders, or tablets disintegrating agents; it can also be as finely divided solid which is in admixture with the finely divided active compound. For the preparation of tablets, the active compound is mixed with carrier having the necessary binding properties in suitable proportions and compacted in the shape and size desired. The powders and tablets preferably contain from about 5 to about 70 percent of the active ingredient. Suitable solid carriers are lactose, pectin, dextrin, starch, gelatin, tragacanth, low melting wax, cocoa butter, and the like. The term “preparation” is intended to include the formulation of the active compound with encapsulating material as carrier providing a capsule in which the active component (with or without other carriers) is surrounded by carrier, which is thus in association with it. Similarly, capsules can be used as solid dosage forms suitable for oral administration.

Liquid form preparations include solutions, suspensions, and emulsions. As an example may be mentioned water or water-propylene glycol solutions for parenteral injection. Such solutions are prepared so as to be acceptable to biological systems (isotonicity, pH, etc.). Liquid preparations can also be formulated in solution in aqueous polyethylene glycol solution. Aqueous solutions suitable for oral use can be prepared by dissolving the active component in water and adding suitable colorants, flavours, stabilizing, and thickening agents as desired. Aqueous suspension suitable for oral use can be made by dispersing the finely divided active component in water with viscous material, i.e., natural or synthetic gums, resins, methyl cellulose, sodium carboxymethyl cellulose, and other well-known suspending agents.

Ointment preparations contain heavy metal salts of a compound of Formula I with a physiologically acceptable carrier. The carrier is desirably a conventional water-dispersible hydrophilic or oil-in-water carrier, particularly a conventional semi-soft or cream-like water-dispersible or water soluble, oil-in-water emulsion infected surface with. a minimum of discomfort. Suitable compositions may be prepared by merely incorporating or homogeneously admixing finely divided compounds with the hydrophilic carrier or base or ointment.

Preferably, the pharmaceutical preparation is in unit dosage form. In such form, the preparation is subdivided into unit doses containing appropriate quantities of the active component. The unit dosage form can be a packaged preparation, the package containing discrete capsules, powders in vials or ampoules, and ointments capsule, cachet, tablet, gel, or cream itself or it can be the appropriate number of any of these packaged forms.

The quantity of active compound in a unit dose of preparation may be varied or adjusted from less than 1 mg to 100 mg according to the particular application and the potency of the active ingredient.

In therapeutic use as agents for treating bacterial infections the compounds utilized in the pharmaceutical method of this invention are administered at the initial dosage of about 3 mg to about 40 mg per kilogram daily. The dosages, however, may be varied depending upon the requirements of the patient and the compound being employed. Determination of the proper dosage for a particular situation is within the smaller dosages which are less than the optimum dose. Small increments until the optimum effect under the daily dosage may be divided and administered in portions during the day if desired.

In order to achieve the above mentioned objects in accordance with the purpose of the invention as embodied and broadly described herein, there are provided process for the synthesis of compounds of Formulae I, II, III and IV. Pharmaceutically acceptable non-toxic acid addition salts of the compounds of the present invention of Formulae I, II, III and IV may be formed with inorganic or organic acids, by methods well known in the art.

The present invention also includes within its scope prodrugs of the compounds of Formulae I, II, III and IV. In general, such prodrugs will be functional derivatives of these compounds which readily get converted in vivo into defined compounds. Conventional procedures for the selection and preparation of suitable prodrugs are known.

The invention also includes pharmaceutically acceptable salts, the enantiomers, diastereomers, N-oxides, prodrugs, metabolites in combination with pharmaceutically acceptable carrier and optionally included excipient.

Other objects and advantages of the invention will be set forth in the description which follows, and in part will be apparent from the description, or may be learned by the practice of the invention. The objects and the advantages of the invention may be released and obtained by means of the mechanism and combination pointed out in the appended claims.

### DETAILED DESCRIPTION OF THE INVENTION

The compounds of the present invention may be prepared by following the reaction sequences as depicted in the schemes defined below.

Mainly five different amines of Formula V

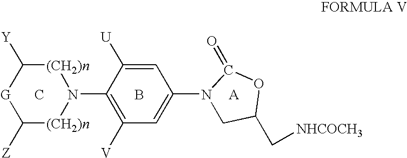

identified as five different cores, namely

-(S)-N-[[3-[3-Fluoro-4-(N-piperazinyl)phenyl]-2-oxo-5-oxazolidinyl]methyl]acetamide (core I);

-(S)-N-[[3-[3-Fluoro[4-[3-(1α,5α,6α)-[N-methyl]amino]-3-azabicyclo-[3.1.0]hexane]phenyl ]-2-oxo-5-oxazolidinyl]methyl]acetamide (core II);

-(S)-N-[[3-[3-Fluoro[4-[3-(1α,5α,6α)-6-[N-methyl]amino methyl]-3-azabicyclo-[3.1.0]hexane]phenyl]-2-oxo-5-oxazolidinyl]methyl]acetamide (core III);

-(S)-N-[[3-[4-[4-N-methylamino piperidin-1-yl]-3-fluorophenyl}-2-oxo-oxazolidin-5-yl]methyl acetamide (core IV); and,

-(S)-N-[[3-[3[Fluoro-4-(N-1-homopiperazinyl)phenyl]-2-oxo-5-oxazolidinyl]-methyl]acetamide (core V),

were used for analoguing purposes.

Key intermediate amines of Formula V for the analogue preparation were prepared from commercially available reagents wherein G in amines of Formula V is defined as NH, CH(NHR), —CH—CH₂NHR wherein R is H, ethyl, methyl, isopropyl, acetyl, cyclopropyl, alkoxy, or acetyl and U, V, Y and Z are as defined for Formula II. Some amines of Formula V are already known in the literature and are given by reference and if they have been made for the first time or by a different procedures or variation of known procedure they are described in detail in the experimental section.

Optically pure amines of Formula V could be obtained either by one of a number of asymetric syntheses or alternatively by resolution from a racemic mixture by selective crystallization of a salt prepared, with an appropriate optically active acid such as dibenzoyl tartrate or 10-camphorsulfonic acid, followed by treatment with base to afford the optically pure amine.

The compounds of the present invention represented by general Formula I may be prepared by the method of reaction in Scheme I:

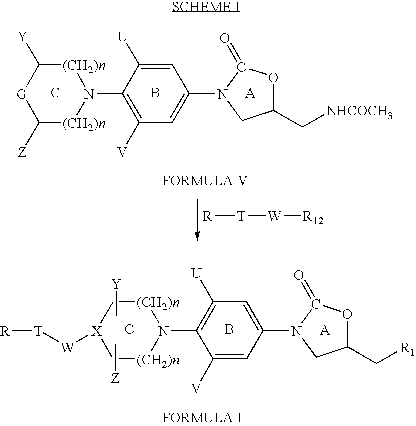

In Scheme I, the heteroaromatic group with the corresponding appendage can be introduced on the nitrogen atom of ring C of compounds of Formula V by one of the methods described below to given Formula I, wherein R₁₂ is a suitable leaving group well known to one of ordinary skill in the art such as fluoro, chloro, bromo, SCH₃, —SO₂CH₃, —SO₂CF₃ or OC₆H₅ etc. and G in amines of Formula V is defined as NH, CH(NHR₁₃), —CH—CH₂NHR₁₃ wherein R₁₃ is H, ethyl, methyl, isopropyl, acetyl, cyclopropyl, alkoxy or acetyl U, V, Y and Z are as defined for Formula I earlier.

Amine of structure of Formula V is reacted with a heteroaromatic compound of Formula R-T-W-R₁₂ wherein R, T, W are the same as defined for Formula I earlier. For the preparation of compounds of Formula I when W is equal to CH₂ corresponding aldehyde can be used through a process of reductive amination and is attached to amine of Formula V.

Similarly, for the preparation of compound of Formula I wherein W is equal to C═O corresponding acid can be used and the amino of Formula V can be acylated through activated esters in the presence of condensing agents such as 1,3-dicyclohexylcarbodiimide (DCC) and 1-(3-dimethylaminopropyl)-3-ethylcarbodiimide (EDC). Other methods of acylation can also be employed.

Alternatively, the compounds having carbonyl link can also be made by reacting heteroaromatic compound of the Formula VI

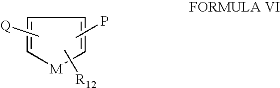

such as N-methyl pyrrole with the intermediate amine of Formula V in the presence of triphosgene or phosgene. Carbonyl linkers may also be introduced between hetero-aromatic compound such as 3-bromothiophene and amine of Formula V with carbon monoxide and the catalyst such as Pd(PPh₃)₂Cl₂. Extended chain pyrroles having dicarbonyl linkers can also be obtained from treatment with oxalyl chloride and amine of the Formula V.

The reduction of the carbonyl linkers using the standard reducing agents results in the formation of methylene linkers.

Preparation of the compound of Formula I as represented by Formula II (where heterocycle is 5 membered ring) is accomplished as exemplified below by three methods A, B and C as shown in Scheme II:

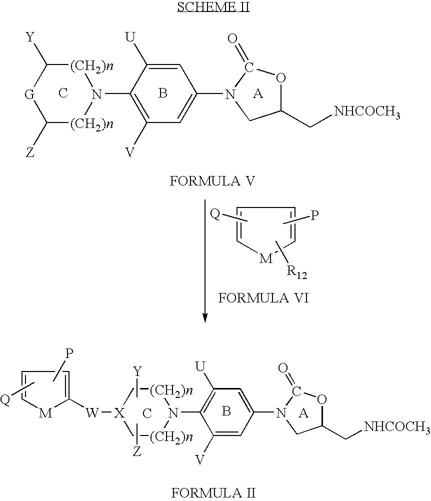

Method A:

Amine of structure V is reacted with a heteroaromatic compound of Formula VI having R₁₂ as a suitable leaving group defined earlier for Scheme I. Q, P and M are as defined for Formula II.

The reaction is done in a suitable solvent such as dimethylformamide, dimethylacetamide, ethanol or ethylene glycol at a suitable temperature in the range of −70° C. to 180° C. to afford compounds of Formula I. The presence of a suitable base such as triethylamine, diisopropyl amine, potassium carbonate, sodium bicarbonate is useful in some cases to improve the yield of the reaction.

Method B:

Reductive alkylation of the amine intermediate of Formula V, with the corresponding heterocyclic aldehydes of the Formula VI, such as furaldehyde (Q, P=H, M=O; R₁₂ is CHO) using known reducing agents well known to one of ordinary skill in the art such as sodium triacetoxyborohydride or sodium cyanoborohydride gave the products of Formula II wherein W=CH₂ as shown in the Scheme II.

Method C:

Acylation of intermediate amines of Formula V with heterocyclic acid of Formula VI, such as 2-furoic acid (Q,P=H; M=O, R₁₂=COOH) gave products of Formula II, wherein W=CO, as shown in the Scheme II wherein U, V, Y, Z, X, W, M, P, Q and R₁₂ are the same.

-(S)-N[[3-[3-Fluoro-4-[N-1[4-(5-nitro-2-thienoyl )]piperazinyl]phenyl]2-oxo-5-oxazolidinyl]methyl]acetamide hydrochloride was prepared using this method.

Alternatively, the compounds having carbonyl link can also be made by reacting heteroaromatic compound of the Formula VI such as N-methyl pyrrole with the intermediate amine of Formula V in the presence of triphosgene or phosgene. Carbonyl linkers may also be introduced between heteroaromatic compound such as 3-bromothiophene and amine of Formula V with carbon monoxide and the catalyst such as Pd(PPh₃)₂Cl₂. Extended chain pyrroles having dicarbonyl linkers can also be obtained from treatment with oxalyl chloride and amine of the Formula V.

The reduction of the carbonyl linkers using the standard reducing agents results in the formation of methylene linkers.

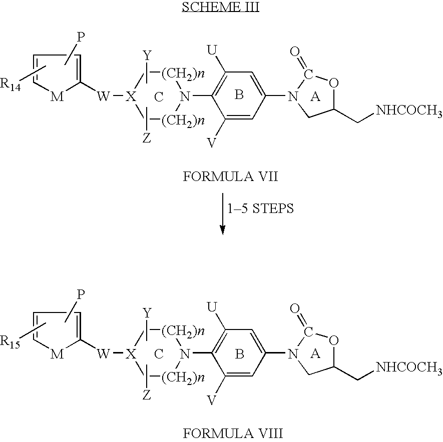

The compounds prepared by Scheme I represented by Formula VII

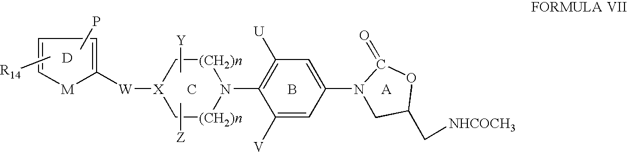

were further used as starting compounds for further derivatisation as represented by Scheme III wherein U,V,Y,Z,X,W,P,Q, n and M are the same as defined earlier. The group R₁₄ was transformed in one to five steps into final compounds of Formula VIII

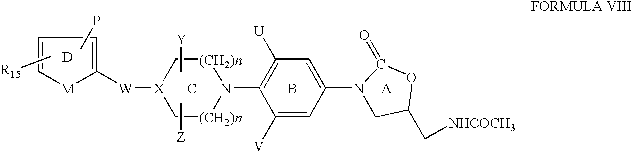

wherein U,V,Y,Z, n, X,W,P and M are the same as defined earlier containing transformed group R₁₅. In most cases the R₁₄ group in starting compounds were compounds containing R₁₄ as aldehyde and ketones.

The following compounds are exemplified in Scheme-IIIA, IIIB and IIIC.

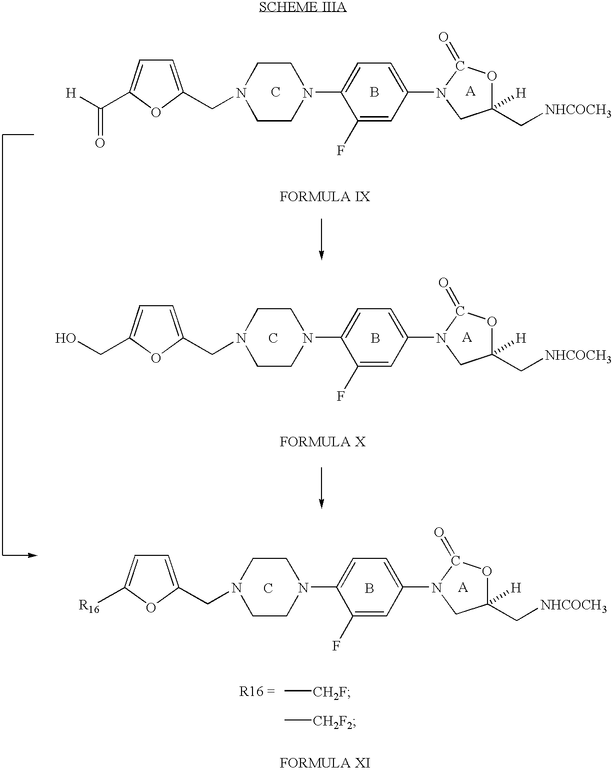

(S)-N-[[3-[3-Fluoro-4-[N-1{2-furyl-[4-(5-hydroxymethyl)methyl}]piperazinyl]-2-oxo-5-oxazolidinyl]methyl]acetamide represented by Formula X was prepared by reducing aldehyde of Formula IX with sodium borohydride.

(S)-N-[[3-[3-fluoro-4-[N-1{2-furyl-[4-(5-fluoromethyl)methyl}]piperazinyl]-2-oxo-5-oxazolidinyl]-methyl]acetamide of Formula XI (R₁₆1=CH₂F) was prepared by reacting (S)-N-[[3-[3-Fluoro-4-[N-1{2-furyl-[4-(5-hydroxymethyl)methyl}]piperazinyl]-2-oxo-5-oxazolidinyl]methyl]acetamide by reacting Formula X with diethylamino sulfurtrifluoride.

(S)-N-[[3-[3-fluoro-4-[N-1{2-furyl-[4-(5-difluoromethyl)methyl}]piperazinyl]-2-oxo-5-oxazolidinyl]-methyl]acetamide of Formula XI (R₁₆═CH₂F₂) was prepared by reacting (S)-N-[[3-Fluoro-4-[N-1[4-{2-furyl(5-formyl)methyl}]piperazinyl]phenyl]-2-oxo-5-oxazolidinyl]methyl]acetamide of Formula IX with diethylamino sulfurtrifluoride as shown in Scheme IIIA.

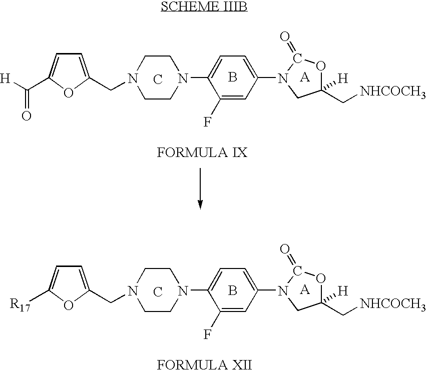

(S)-N-[[3-Fluoro-4-[N-1[4-{2-furyl(5-formyl)methyl}]piperazinyl]phenyl]-2-oxo-5-oxazolidinyl]methyl]acetamide of Formula IX was reacted with hydroxylamine and hydrazine hydrate to give (S)-N-[[3-[3-Fluoro-4-[N-1-[4-(2-furyl-(5-aldoxime)methyl}]piperazinyl]phenyl]-2-oxo-5-oxazolidinyl]methyl]acetamide of Formula XII (R₁₇═) and (S)-N-[[3-[3-Fluoro-4[N-1-[4-{2-furyl-(5-hydrazone)-methyl}]piperazinyl]-phenyl]-2-oxo-5-oxazolidinyl]-methyl]acetamide of Formula XII

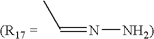

(S)-N-[[3-[3-Fluoro-4-[N-1[4-{2-furyl(5-aldoxime(methyl-4-(N-carboxyaminophenylacetate)methyl}]piperazinyl]phenyl]-2-oxo-5-oxazolidinyl]methyl]acetamide of Formula XII

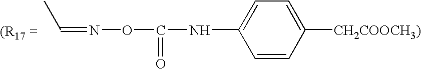

was made starting from (S)-N-[[3-[3-Fluoro-4-[N-1-[4-(2-furyl-(5-aldoxime)methyl}]piperazinyl]phenyl]-2-oxo-5-xazolidinyl]methyl]acetamide of Formula XII

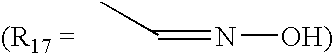

and reacting with isocyanate.

(S)-N-[[3-[3-Fluoro-4-[N-1[4-{2-furyl(5-cyano)methyl}]piperazinyl]phenyl]-2-oxo-5-oxazolidinyl]methyl]acetamide of Formula XII (R₁₇═CN) was prepared from (S)-N-[[3-[3-Fluoro-4-[N-1[4-{2-furyl(5-aldoxime)methyl}]phenyl]-2-oxo-5-oxazolidinyl]methyl]acetamide of Formula XII

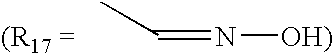

by the use of anhydride and triethylamine.

(S)-N-[[3-Fluoro-4-[N-1[5-(1,3-dioxane)-2-furylmethyl]piperazinyl]phenyl]-2-oxo-5-oxazolidinyl)methyl]acetamide of Formula XII

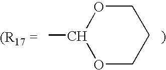

was made using (S)-N-[[3-Fluoro-4-[N-1[4-{2-furyl(5-formyl)methyl}]piperazinyl]phenyl]-2-oxo-5-oxazolidinyl]methyl]acetamide of Formula IX with 1,3-propane diol and BF₃ etherate.

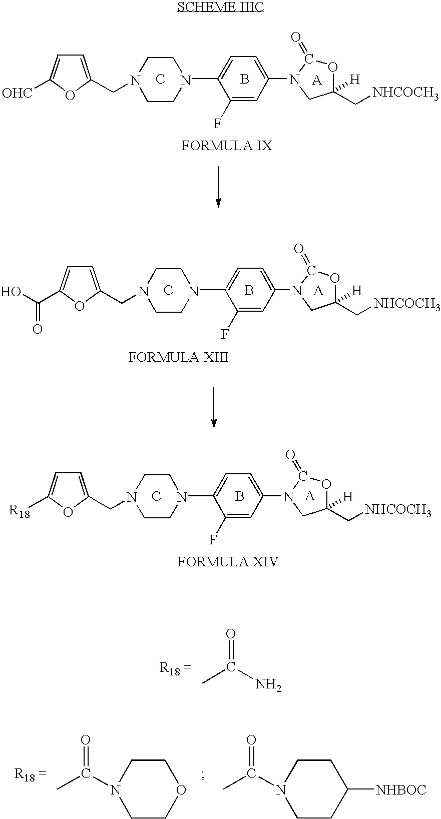

(S)-N-[[3-Fluoro-4-[N-1[4-{2-furyl(5-carboxy)methyl}]piperazinyl]phenyl]-2-oxo-5-oxazolidinyl]methyl]acetamide of Formula XIII was made using (S)-N-[[3-Fluoro-4-[N-1[4-(2-furyl(5-formyl)methyl}]piperazinyl]phenyl]-2-oxo-5-oxazolidinyl]methyl]acetamide of Formula IX by oxidation with Ag₂O.

[[3-Fluoro-4-[N-1[5-(formamido)-2-furylmethyl]piperazinyl]phenyl]-2-oxo-5-oxazolidinyl]methyl]acetamide of Formula XIV

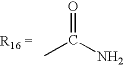

was made by reacting (S)-N-[[3-Fluoro-4-[N-1[4-(2-furyl-(5-carboxyethyl)methyl)piperazinyl]phenyl]-2-oxo-5-oxazolidinyl]methyl]acetamide with aqueous ammonia.

(S)-N-[[3-Fluoro-4-[N-1[5-(4-(tert butoxy carbonyl)amino piperidine)-2-furylmethyl]piperazinyl]phenyl]-2-oxo-5-oxazolidinyl]methyl]acetamide of Formula XIV

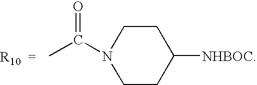

was made by reacting (S)-N-[[3-Fluoro-4-[N-1[4-(2-furyl-(5-carboxy)methyl)piperazinyl]phenyl]-2-oxo-5-oxazolidinyl]methyl]acetamide of Formula XIII with thionyl chloride and 4-(tert butoxy carbonyl)amino piperidine.

(S)-N-[[3-Fluoro-4-[N-1[5-(morpholine-1-carbonyl)-2-furylmethyl]piperazinyl)-phenyl]-2-oxo-5-oxazolidinyl]methyl]acetamide of Formula XIV

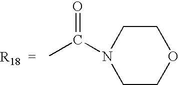

was made by reacting (S)-N-[[3-Fluoro-4-[N-1[4-(2-furyl-(5-carboxy)methyl)-piperazinyl]phenyl]-2-oxo-5-oxazolidinyl]methyl]acetamide of Formula XIII with morpholine in the presence of oxalyl chloride.

The transformations effected are described in the experimental section. In the above synthetic methods where specific acids, bases, solvents, catalysts, oxidising agents, reducing agents etc. are mentioned, it is to be understood that the other acids, bases, solvents, catalysts, oxidising agents, reducing agents etc. may be used. Similarly, the reduction temperature and duration of the reaction may be adjusted according to the need. An illustrative list of particular compounds according to the invention and capable of being produced by the above mentioned schemes include:

Chemical Name Compound No.

1. (S)-N-[[3-[3-Fluoro-4-[N-1-[4-(2-furoyl)piperazinyl]]phenyl]-2-oxo-5-oxazolidinyl]methyl]acetamide

2. (S)-N-[[3-[3-Fluoro-4-[N-1[4-{2-furyl(5-formyl)methyl}]piperazinyl]phenyl]-2-oxo-5-oxazolidinyl]methyl]acetamide

3. (S)-N-[[3-Fluoro-4-[N-1[4-(2-furyl-(5-carboxyethyl)methyl)piperazinyl]phenyl]-2-oxo-5-oxazolidinyl]methyl]acetamide

4. (S)-N-[[3-Fluoro-4-[N-1[4-(5-bromo-2-furoyl)]piperazinyl]phenyl]-2-oxo-5-oxazolidinyl]methyl]acetamide

5. (S)-N-[[3-Fluoro-4-[N-1[4-(5-chloromethyl-2-furoyl)piperazinyl]phenyl]-2-oxo-5-oxazolidinyl]methyl]acetamide

6. (S)-N-[[3-Fluoro-4-[N-1[4-(5-nitro-2-furoyl)piperazinyl]phenyl]-2-oxo-5-oxazolidinyl]methyl]acetamide

7. (S)-N[[3-[3-Fluoro-4-[N-1[4-{2-(2-thienyl)dicarbonyl}]piperazinyl]phenyl]2-oxo-5-oxazolidinyl]methyl]acetamide

8. (S)-N[[3-[3-Fluoro-4-[N-1[4-(3-furoyl)]piperazinyl]phenyl]2-oxo-5-oxazolidinyl]-methyl]acetamide

9. (S)-N[[3-[3-Fluoro-4-[N-1[4-{2-furyl(5-bromo)methyl}]piperazinyl]phenyl]2-oxo-5-oxazolidinyl]methyl]acetamide

10. (S)-N[[3-[3-Fluoro-4-[N-1[4-{2-thienyl(5-chloro)methyl}]piperazinyl]phenyl]2-oxo-5-oxazolidinyl]methyl]acetamide

11. (S)-N[[3-[3-Fluoro-4-[N-1[4-(2-furylmethyl)]piperazinyl]phenyl]2-oxo-5-oxazolidinyl]methyl]acetamide

12. (S)-N-[[3-[3-Fluoro-4-[N-1[4-(2-thienylmethyl)]piperazinyl]phenyl]-2-oxo-5-oxazolidinyl]methyl]acetamide

13. (S)-N[[3-[3-Fluoro-4-[N-1[4-(2-thienylacetyl)]piperazinyl]phenyl]2-oxo-5-oxazolidinyl]methyl]acetamide

14. (S)-N-[[3-[3-Fluoro-4-[N-1[4-{2-thienyl(4-bromo)methyl}]piperazinyl]phenyl]-2-oxo-5-oxazolidinyl]methyl]acetamide

15. (S)-N-[[3-[3-fluoro-4-[N-1-[4-{2-furyl-(5-nitro)methyl}]piperazinyl]phenyl]-2-oxo-5-oxazolidinyl]methyl]acetamide

16. Hydrochloride salt of (S)-N-[[3-Fluoro-4-[N-1[4-{2-furyl(5-nitro)methyl}]piperazinyl]phenyl]-2-oxo-5-oxazolidinyl]methyl]acetamide

17. Citrate salt of (S)-N-[[3-Fluoro-4-[N-1[4-{2-furyl(5-nitro)methyl}]piperazinyl]phenyl]-2-oxo-5-oxazolidinyl]methyl]acetamide

18. (S)-N[[3-[3-Fluoro-4-[N-1[4-(2-pyrrolylmethyl)]piperazinyl]phenyl]2-oxo-5-oxazolidinyl]methyl]acetamide

19. (S)-N[[3-[3-Fluoro-4-[N-1[4-{2-thienyl(3-methyl)methyl}]piperazinyl]phenyl]2-oxo-5-oxazolidinyl]methyl]acetamide

20. (S)-N[[3-[3-Fluoro-4-[N-1[4-(3-furylmethyl)]piperazinyl]phenyl]2-oxo-5-oxazolidinyl]methyl]acetamide

21. (S)-N[[3-[3-Fluoro-4-[N-1[4-{2-thienyl(5-methyl)methyl}]piperazinyl)phenyl]2-oxo-5-oxazolidinyl]methyl]acetamide

22. (S)-N[[3-[3-Fluoro-4-[N-1[4-{2-pyrrole(1-methyl)methyl}]piperazinyl]phenyl]2-oxo-5-oxazolidinyl]methyl]acetamide

23. (S)-N[[3-[3-Fluoro-4-[N-1[4-(2-thienyl(5-nitro)methyl}]piperazinyl]phenyl]2-oxo-5-oxazolidinyl]methyl]acetamide

24. (S)-N[[3-[3-Fluoro-4-[N-1[4-[2-furyl {5-(N-thiomorpholinyl)methyl}methyl]piperazinyl]phenyl]2-oxo-5-oxazolidinyl]methyl]acetamide

25. (S)-N[[3-[3-Fluoro-4-[N-1[4-[2-furyl {5-(N-morpholinyl)methyl}methyl]]piperazinyl]phenyl]2-oxo-5-oxazolidinyl]methyl]acetamide

26. (S)-N-[[3-Fluoro-4-[N-1[4-{2-furyl(5-acetoxymethyl)methyl}]piperazinyl]phenyl]-2-oxo-5-oxazolidinyl]methyl]acetamide

27. (S)-N-[[3-Fluoro-4-[N-1[4-{2-thienyl(5-bromo)methyl}]piperazinyl]phenyl]-2-oxo-5-oxazolidinyl]methyl]acetamide

28. (S)-N-[[3-Fluoro-4-[N-1[4-(5-nitro-2-furylmethyl)piperazinyl]phenyl]-2-oxo-oxazolidinyl]methyl]dichloroacetamide

29. (S)-N[[3-[3-Fluoro-4-[N-1[4-(5-nitro-2-thienoyl)]piperazinyl]phenyl]2-oxo-5-oxazolidinyl]methyl]acetamide hydrochloride

30. (S)-N[[3-[3-Fluoro-4-[N-1[4-(2′,2′-diphenyl-2′-hydroxyacetyl)]piperazinyl]phenyl]2-oxo-5-oxazolidinyl]methyl]acetamide

31. (S)-N-[[3-[3-Fluoro[4-[3-(1α,5α,6α)-6-[N-(5-nitro-2-furoyl)-N-methyl]amino]-3-aza-bicyclo-[3.1.0]hexane]phenyl]-2-oxo-5-oxazolidinyl]methyl]acetamide

32. (S)-N-[[3-[3-Fluoro[4-[3-(1α,5α,6α)-6-[N-(3-furoyl)-N-methyl]amino]-3-azabicyclo-[3.1.0]hexane]phenyl]-2-oxo-5-oxazolidinyl]methyl]acetamide

33. (S)-N-[[3-[3-Fluoro[4-[3-(1α,5α,6α)-6-[N-(5-bromo-2-furoyl)-N-methyl]amino]-3-azabicyclo-[3.1.0.]hexane]phenyl]-2-oxo-5-oxazolidinyl]methyl]acetamide

34. (S)-N-[[3-[3-Fluoro[4-[3-(1α,5α,6α)-6-[N-(5-nitro-2-thienylmethyl)-N-methyl]-amino]-3-azabicyclo-[3.1.0]hexane]phenyl]-2-oxo-5-oxazolidinyl]methyl]acetamide

35. (S)-N-[[3-[3-Fluoro[4-[3-(1α,5α,6α)-6-[N-(5-nitro-2-furylmethyl)-N-methyl]amino]-3-azabicyclo-[3.1.0]hexane]phenyl]-2-oxo-5-oxazolidinyl]methyl]acetamide

36. (S)-N-[[3-[3-Fluoro[4-[3-(1α,5α,6α)-6-[N-(5-formyl-2-furylmethyl)-N-methyl]amino-methyl]-3-azabicyclo-[3.1.0]hexane]phenyl]-2-oxo-5-oxazolidinyl]methyl]acetamide

37. (S)-N-[[3-[3-Fluoro[4-[3-(1α,5α,6α)-6-[N-(5-carboxyethyl-2-furylmethyl)-N-methyl]aminomethyl]-3-azabicyclo-[3.1.0]hexane]phenyl]-2-oxo-5-oxazolidinyl]methyl]acetamide

38. (S)-N-[[3-[3-Fluoro[4-[3-(1α,5α,6α)-6-[N-(2-thiopheneacetyl)-N-methyl]amino-methyl]-3-azabicyclo-[3.1.0]hexane]phenyl]-2-oxo-5-oxazolidinyl]methyl]acetamide

39. (S)-N-[[3-[3-Fluoro[4-[3-(1α,5α,6α)-6-[N-(5-nitro-2-thienylmethyl)-N-methyl]-amino-methyl]-3-azabicyclo-[3.1.0]hexane]phenyl]-2-oxo-5-oxazolidinyl]methyl]acetamide

40. (S)-N-[[3-[3-Fluoro[4-[3-(1α,5α,6α)-6-[N-(5-nitro-2-furylmethyl)-N-methyl]amino-methyl]-3-azabicyclo-[3.1.0]hexane]phenyl]-2-oxo-5-oxazolidinyl]methyl]acetamide

41. (S)-N-[[3-[4-[4-(N-methyl-N-2furyl(5formyl)methylaminopiperidine-1-yl]-3-fluorophenyl]-2-oxo-oxazolidin-5-yl]methyl]acetamide

42. (S)-N-[[3-[4-[4-(N-methyl-N-(3,5-difluorobenzoyl)aminopiperidine-1-yl]-3-fluorophenyl]-2-oxo-oxazolidin-5-yl]methyl]acetamide

43. (S)-N-[[3-[4-[4-(N-methyl-N-(5-bromo-2-furoyl)aminopiperidine-1-yl]-3-fluorophenyl]-2-oxo-oxazolidin-5-yl]methyl]acetamide

44. (S)-N-[[3-[4-[4-(N-methyl-N-(5-nitro-2-furoyl)aminopiperidine-1-yl]-3-fluoro-phenyl]-2-oxo-oxazolidin-5-yl]methyl]acetamide

45. (S)-N-[[3-[4-[4-(N-methyl-N-3-furoyl)aminopiperidine-1-yl]-3-fluorophenyl]-2-oxo-oxazolidin-5-yl]methyl]acetamide

46. (S)-N-{3-[4-[4-(N-methyl, N-2-furoyl)aminopiperidine-1-yl]-3-fluorophenyl]-2-oxo-oxazolidin-5-yl]methyl]acetamide

47. (S)-N-{3-[4-[4-(N-methyl,2-thiopheneacetyl)aminopiperidine-1-yl]-3-fluorophenyl]-2-oxo oxazolidin-5-yl methyl]acetamide

48. (S)-N-[[3-[4-[4-(N-methyl-N-2furylmethyl)aminopiperidine-1-yl]-3-fluorophenyl]-2-oxo-oxazolidin-5-yl]methyl]acetamide

49. (S)-N-[[3-[4-[4-(N-methyl-N-3-furyl)aminopiperidine-1-yl]-3-fluorophenyl]-2-oxo-oxazolidin-5-yl]methyl]acetamide

50. (S)-N-[[3-[4-[4-(N-methyl-N-2-furyl(5-nitro)methyl)aminopiperidine-1-yl]-3-fluorophenyl]-2-oxo-oxazolidin-5-yl]methyl]acetamide

51. (S)-N-[[3-[4-[4-(N-methyl-N-2-thienyl(5-nitro)methyl)aminopiperidine-1-yl]-3-fluorophenyl]-2-oxo-oxazolidin-5-yl]methyl]acetamide

52. (S)-N-[[3-[4-[4-(N-methyl-N-2-thienylmethyl)aminopiperidine-1-yl]-3-fluorophenyl]-2-oxo-oxazolidin-5-yl ]methyl]acetamide

53. (S)-N-[[3-[4-[4-(N-methyl-N-(5-methyl-2-thienylmethyl)aminopiperidine-1-yl]-3-fluorophenyl]-2-oxo-oxazolidin-5-yl]methyl]acetamide

54. (S)-N-{3-[4-[4-(N-methyl,2-(5-bromo)thienylmethyl)aminopiperidine-1-yl]-3-fluorophenyl]-2-oxo-oxazolidin-5-yl methyl)acetamide

55. (S)-N[[3-[3-Fluoro-4-[N-1[4-{2-furyl(5-formyl)methyl}]homopiperazinyl]phenyl]2-oxo-5-oxazolidinyl]methyl]acetamide

56. (S)-N[[3-[3-Fluoro-4-[N-1[4-(2-thienylacetyl)]homopiperazinyl]phenyl]2-oxo-5-oxazolidinyl]methyl]acetamide

57. (S)-N[[3-[3-Fluoro-4-[N-1[4-{2-thienyl(5-nitro)methyl}]homopiperazinyl]phenyl]2-oxo-5-oxazolidinyl]methyl]acetamide

58. (S)-N[[3-[3-Fluoro-4-[N-1[4-(3-furylmethyl)]homopiperazinyl]phenyl]2-oxo-5-oxazolidinyl]methyl]acetamide

59. (S)-N-[[3-[3-fluoro-4-[N-1{2-furyl-[4-(5-difluoromethyl)methyl}]piperazinyl]-2-oxo-5-oxazolidinyl]-methyl]acetamide

60. (S)-N-[[3-[3-Fluoro-4-[N-1-[4-(2-furyl-(5-aldoxime)methyl}]piperazinyl]phenyl]-2-oxo-5-oxazolidinyl]methyl]acetamide

61. (S)-N-[[3-[3-Fluoro-4-[N-1[4-{2-furyl(5-aldoxime(methyl-4-(N-carboxyaminophenyl acetate)methyl}]piperazinyl]phenyl]-2-oxo-5-oxazolidinyl]methyl]acetamide

62. (S)-N-[[3-[3-Fluoro-4[N-1-[4-{2-furyl-(5-hydrazone)-methyl}]-piperazinyl]-phenyl]-2-oxo-5-oxazolidinyl]-methyl]acetamide

63. (S)-N-[[3-[3-Fluoro-4-[N-1{2-furyl-[4-(5-hydroxymethyl)methyl}]piperazinyl]-2-oxo-5-oxazolidinyl]methyl]acetamide

64. (S)-N-[[3-[3-Fluoro-4-[N-1[4-{2-furyl(5-cyano)methyl}]piperazinyl]phenyl]-2-oxo-5-oxazolidinyl]methyl]acetamide

65. (S)-N-[[3-Fluoro-4-[N-1[4-}2-furyl(5-carboxy)methyl}]piperazinyl]phenyl]-2-oxo-5-oxazolidinyl]methyl]acetamide

66. (S)-N-[[3-Fluoro-4-[N-1[5-(1,3-dioxane)-2-furylmethyl]piperazinyl]phenyl]-2-oxo-5-oxazolidinyl]methyl]acetamide

67. (S)-N-[[3-Fluoro-4-[N-1[5-(formamido)-2-furylmethyl]piperazinyl]phenyl]-2-oxo-5-oxazolidinyl]methyl]acetamide

68. (S)-N-[[3-Fluoro-4-[N-1[5-(morpholine-1-carbonyl)-2-furylmethyl]piperazinyl]-phenyl]-2-oxo-5-oxazolidinyl]methyl]acetamide

69. (S)-N-[[3-Fluoro-4-[N-1[5-(4-(tert butoxy carbonyl)amino piperidine)-2-furylmethyl]piperazinyl]phenyl]-2-oxo-5-oxazolidinyl]methyl]acetamide

70. (S)-N-[[3-Fluoro-4-[N-1[4-{(Z)-2-methoxyimino-2-(2-furyl)acetyl}]piperazinyl]-phenyl]-2-oxo-5-oxazolidinyl]methyl]acetamide

71. (S)-N-[[3-[3-Fluoro[4-[3-(1α,5α,6α)-6-[N-(2-thiopheneacetyl)-N-methyl]amino]-3-azabicyclo-[3.1.0]hexane]phenyl]-2-oxo-5-oxazolidinyl]methyl]acetamide

72. (S)-N-[[3-[3-Fluoro[4-[3-(1α,5α,6α)-6-[N-(5-formyl-2-furylmethyl)-N-methyl]-amino]-3-azabicyclo-[3.1.0]hexane]phenyl]-2-oxo-5-oxazolidinyl]methyl]acetamide

73. (S)-N-[[3-[3-Fluoro[4-[3-(1α,5α,6α)-6-[N-(3-thienoyl)-N-methyl]amino]-3-aza-bicyclo[3.1.0]hexane]phenyl]-2-oxo-5-oxazolidinyl]methyl]acetamide

74. (S)-N-[[3-[3-fluoro-4-[N-1{2-furyl-[4-(5-fluoromethyl)methyl}]piperazinyl]-2-oxo-5-oxazolidinyl]-methyl]acetamide

Pharmacological Testing

The compounds of the invention display antibacterial activity when tested by the agar incorporation method. The following minimum inhibitory concentrations (μg/ml) were obtained for representative compounds of the invention which are given below in the following tables.

Guide to Table Abbreviations:

1) S. aureus ATCC 25923—Staphylococus aureus ATCC 25923

2) MRSA 15187—Methicillin Resistant Staphylococcus aureus

3) Ent. faecalis ATCC 29212—Enterococcus faecalis ATCC 29212

4) Ent. faecium 6A—Enterococcus faecium 6A Van®, Cipro®

5) Strep. pne. ATCC 6303—Streptococcus pneumoniae ATCC 6303

6) Strep. pyog. ATCC 19615—Streptococcus pyogenes

7) S. epidermidis—Staphylococcus epidermidis ATCC 12228

TABLE 1
MIC of compounds and standard antibiotics against important pathogens

| Compound | S. aureus | MRSA | MRSA | MRSA | E. faecalis | VRE | S. pyogenes | S. pneum | S. pneum |
|---|---|---|---|---|---|---|---|---|---|
| No. | 25923 | 15187 | 562 | 33 | 29212 | 6A | 19615 | 6303 | AB 34 |
| 02 | 1 | 2 | 1 | 2 | 8 | 8 | 8 | 4 | 8 |
| 14 | 4 | 2 | 2 | 2 | 2 | 2 | 2 | 4 | 4 |
| 60 | 2 | 2 | | 2 | 8 | 16 | 4 | 8 | 8 |
| 66 | 2 | 2 | 2 | 2 | 16 | 16 | 8 | 8 | 8 |
| 12 | 8 | 8 | 8 | 8 | 8 | 8 | 4 | 8 | 8 |
| 62 | 2 | 2 | 1 | 2 | 8 | 8 | 8 | 8 | 8 |
| 61 | 8 | 8 | | 8 | 8 | 8 | 8 | 8 | 8 |
| 15 | 2 | 2 | 2 | 2 | 2 | 1 | 1 | 1 | 2 |
| 01 | 2 | 2 | | 4 | 2 | 2 | 1 | 2 | 1 |
| 27 | 2 | 2 | 2 | 2 | 2 | 4 | 1 | 2 | 0.5 |
| 16 | 2 | 4 | | 4 | 2 | 4 | 0.5 | 1 | 1 |
| 17 | 2 | 2 | | 2 | 2 | 4 | 1 | 2 | 1 |
| 71 | 8 | 8 | | 8 | 8 | 8 | 4 | 4 | 2 |
| 04 | 2 | 2 | | 2 | 2 | 1 | 2 | 1 | 1 |
| 05 | 16 | 4 | | 4 | 8 | 8 | 4 | 4 | 4 |
| 06 | 1 | 0.5 | | 0.5 | 1 | 1 | 2 | 2 | 2 |
| 10 | 8 | 4 | | 4 | 8 | 4 | 1 | 4 | 8 |
| 23 | 8 | 8 | | 8 | 8 | 8 | 1 | 8 | 8 |
| 33 | 4 | 4 | | 4 | 4 | 4 | 0.5 | 8 | 4 |
| 73 | 8 | 8 | | 8 | 8 | 8 | 1 | 4 | 8 |
| 72 | 8 | 4 | | 4 | 8 | 8 | 0.25 | 4 | 4 |
| 32 | 8 | 8 | | 8 | 8 | 8 | 1 | 4 | 4 |
| 08 | 2 | 1 | 1 | 1 | 1 | 1 | 1 | 2 | 4 |
| 07 | 2 | 2 | 2 | 2 | 2 | 2 | 0.5 | 2 | 2 |
| 34 | 1 | 1 | 1 | 1 | 1 | 1 | 2 | 4 | 4 |
| 71 | 4 | 4 | | 4 | 4 | 4 | 2 | 4 | 4 |
| 29 | 0.25 | <0.1 | <0.1 | <0.1 | 1 | 0.5 | 1 | 1 | 1 |
| 44 | 0.25 | <0.1 | <0.1 | <0.1 | 0.25 | <0.1 | 0.5 | 2 | 2 |
| 50 | 1 | 1 | 0.5 | 1 | 0.5 | 0.25 | 0.125 | 0.5 | 2 |
| 40 | 2 | 2 | 1 | 2 | 2 | 4 | 0.125 | NG | 2 |
| 51 | 2 | 1 | 1 | 1 | 4 | 4 | 0.5 | NG | 2 |
| 22 | 2 | 8 | 4 | 8 | 8 | 8 | 1 | 1 | 1 |
| 38 | 4 | 4 | 4 | 4 | 4 | 4 | 1 | 1 | 1 |
| 39 | 8 | 8 | 8 | 4 | 2 | 8 | 0.5 | 0.5 | 0.5 |
| Linezolid | 2 | 1 | 2 | 2 | 2 | 2 | 2 | 2 | 4 |
| Vancomycin | 1 | 0.5 | 0.5 | 0.5 | 4 | >16 | 0.5 | 0.5 | 0.25 |
| Linezolid | 2 | 1 | 2 | 2 | 2 | 2 | 2 | 2 | 4 |
| Vancomycin | 1 | 0.5 | 0.5 | 0.5 | 4 | >16 | 0.5 | 0.5 | 0.25 |

TABLE 2
Summary of in vitro Activity (MIC):

| | | | | | | | | Compound | | Compound | |
|---|---|---|---|---|---|---|---|---|---|---|---|
| | | Vancomycin | | Linezolid | | Penicillin G | | No. 29 | | No. 44 | |
| Bacteria | Number | MIC₅₀ | MIC₉₀ | MIC₅₀ | MIC₉₀ | MIC₅₀ | MIC₉₀ | MIC₅₀ | MIC₉₀ | MIC₅₀ | MIC₉₀ |
| G +ve | 49 | 0.5 | 16 | 2 | 4 | 4 | 32 | 1 | 1 | 0.5 | 2 |
| S. aureus | 8 | 1 | 1 | 2 | 4 | — | — | 0.064 | 0.25 | 0.064 | 0.25 |
| E. faecalis | 7 | 16 | 16 | 4 | 16 | — | — | 1 | 1 | 0.064 | 0.25 |
| S. pneumoniae | 19 | 0.5 | 0.5 | 0.5 | 2 | 4 | 32 | 1 | 1 | 1 | 2 |

| | Compound no. | | Compound No. | | Compound No. | | Compound No. | |
|---|---|---|---|---|---|---|---|---|
| | 15 | | 06 | | 50 | | 40 | |
| Bacteria | MIC₅₀ | MIC₉₀ | MIC₅₀ | MIC₉₀ | MIC₅₀ | MIC₉₀ | MIC₅₀ | MIC₉₀ |
| G +ve | 1 | 2 | 1 | 2 | 1 | 2 | 1 | 2 |
| S. aureus | 2 | 2 | 1 | 1 | 1 | 2 | 2 | 4 |
| E. faecalis | 2 | 2 | 1 | 1 | 0.5 | 1 | 2 | 4 |
| S. pneumoniae | 2 | 4 | 2 | 2 | 1 | 2 | 1 | 1 |

TABLE 3
Geometric Mean of in vitro activity(MIC):

| | | | | Compound | Compound | Compound | Compound | Compound |
|---|---|---|---|---|---|---|---|---|
| | Vancomycin | Linezolid | Penicillin G | No. 44 | No. 15 | No. 06 | No. 50 | No. 40 |
| Gram + ve | 0.93 | 1.25 | 0.76 | 0.31 | 1.08 | 0.79 | 0.72 | 0.93 |
| S. aureus | 1 | 2.38 | — | 0.11 | 1.63 | 0.74 | 1 | 1.83 |
| E. faecalis | 9.75 | 4 | — | 0.11 | 1.6 | 0.92 | 0.42 | 2.38 |
| S. pneumoniae | 0.39 | 0.60 | 2.16 | 0.96 | 1.04 | 1.27 | 1.41 | 0.71 |

TABLE 4
Changes in MIC under different conditions

Broth MIC

| | | Normal | +50% |
|---|---|---|---|
| Compound No. | Agar MIC | MH broth | Sheep serum |
| 16 | 2 | 2 | 2 |
| 08 | 1 | 2 | 4 |
| 29 | <0.1 | 0.25 | 0.5 |
| 44 | <0.1 | <0.1 | 0.25 |
| Linezolid | 2 | 1 | 2 |
| Vancomycin | 1 | 1 | 1 |

TABLE 5
Linezolid has 30% protein binding
In vitro and in vivo activity against MRSA 562

| | | ED50 (mg/kg |
|---|---|---|
| RBx | MIC (μg/ml) | body weight) PO |
| Vancomycin | 0.5 | 8.84 (IV) |
| Linezolid | 2 | 4.56 |
| 67 | 2 | >25 |
| 15 | 2 | 4.33 |
| 04 | | >25 |
| 06 | 1 | >25 |
| 08 | 1 | 25 |
| 71 | 4 | >25 |
| 29 | <0.1 | >25 |
| 44 | <0.1 | >25 |
| 50 | 0.5 | |
| 07 | 2 | >25 |

The in vitro antibacterial activity of the compounds were demonstrated by the agar incorporation method National Committee for Chemical Laboratory Standards (NCCLS M 7 and M 100-S8 documents). Briefly, the compounds were dissolved in DMSO and doubling dilution of the compounds were incorporated into Meuller Hilton agar before solidification. Inoculum was prepared by suspending 4 to 5 colonies into 5 ml of normal saline solution and adjusting the turbility to 0.5 Macfarland turbidity standard tables (1.5×10⁸ CFU/ml), after appropriate dilutions, 10⁴ CFU/spot was transferred into the surface of dried plate and incubated for 18 hours (24 hours for MRSN studies). The concentration showing no growth of the inoculated culture was recorded as the MIC. Appropriate ATCC standard strains were simultaneously tested and result recorded only when the MIC's against standard antibiotics were within the acceptable range.

Activity Against Anaerobes and Microbacterium

Since the time of Loius Pasteur's isolation and discription of Clostridium septicum and his use of the term “anaerobies” for organisms that did not require oxygen for growth, there has been increasing recognition of the role of anaerobic bacteria in human disease and their pathogenic potential. Much has been learned about their associated virulence factors and wide spectrum of anaerobic infections caused by both invasion and intoxication. Even so, new anaerobic infections (e.g. diarrhoea due to toxigenic Bacteroides fragilis and Anaerobiosperillum succiniciproducens) and the description of new pathogenic anaerobic species (e.g. Bilophila wadsworthia from abdominal infection, Fusobacterium ulcerans from skin ulcers, Prevotella heparinolytica and B. tectum from animal bite wounds and B. forsythus from periodontal infection) have enhanced the clinical frontiers of anaerobic bacteriology.

Therapy for many anaerobic infections has always required appropriate antimicrobial therapy coupled with surgical debridement or drainage. In the late 1970s and 1980s, a variety of antimicrobial agents (particularly β lactam agents such as cefoxitin, imipenem and a β lactamase inhibitor combination) were developed to supplement the basic antimicrobial armamentorium of metronidazole and chloramphenicol. However, as the rate of resistance of bacteria, including anaerobes, to many of these commonly used antimicrobial agents has increased, clinicians are once again focusing on antimicrobial therapy and searching for enhanced agents or new classes of therapeutic agents with anti-anaerobic activity.

Antibiotic resistance among anaerobes has increased steadily over the last several years, leaving the clinician with a limited number of potent antimicrobials from which to choose.

The most important anaerobes clinically are the genera of gram negative rods. Bacteroides, especially the B. fragilis group is particularly important. The other principal gram negative genera are Prevotella, Fusobacterium, Porphyromonas, Bilophila and Sitterella. Among the gram positive anaerobes, there are cocci (primarily Peptostreptococcus) and spore forming (clostridium) and non spore forming bacilli (Actinomyces and Propionibacteria).

Treatment of anaerobic infections may be difficult. Failure to provide coverage for anaerobes in mixed infections may lead to a poor response or to no response. Many antibacterial agents including aminoglycosides, trimethoprimsulphamethoxazole, most quinolones and monobactams have poor activity against many or most anaerobes. Four groups of drug are active against majority of anaerobic bacteria of clinical significance: these are nitroimidazole such as metronidazole, carbepenems such as imipenem, chloramphenicol and a combination of β lactam and β lactamase inhibitors.

Non spore forming, anaerobic, gram positive bacilli (e.g. Actinomyces, Eubacterium and Propionibacterium) are commonly resistant to metronidazole. Of late, there has been reports of resistance to all the above agents in small number of strains of B. fragilis group. Cefoxitin, clindamycin and broad spectrum penicillins such as ticarcillin or piperacillin also have some anti anaerobic activity. But 15-25% of B. fragilis isolated in the U.S. hospitals are resistant to these drugs. Cefoxitin and clindamycin have relatively weak activity against clostridia other than C. perfringens (20-35% of such strains re resistant) and some anaerobic cocci are resistant to clindamycin. Penicillin G is not reliable for treating serious infections involving any of these anaerobic gram negative bacilli because the incidence of β lactamase production among these organisms is high. Consequently, there is a need to discover and develop a new agent active against all anaerobes including drug resistant strains.

1. Agar Dilution Method for Anaerobic Bacteria:

MICs were determined by the NCCLS agar dilution method with Wilkins Chalgren Agar (Difco). The plates were incubated in an anaerobic jar containing an atmosphere of 85% nitrogen, 10% hydrogen and 5% carbon dioxide for 48 hour.

| Antibiotics | MIC₅₀ | MIC₉₀ | Geometric Mean | MIC Range |
|---|---|---|---|---|
| Compound No. 16 | 0.032 | 0.25 | 0.037 | 0.004-1 |
| Linezolid | 1 | 4 | 1.134 | 0.25-4 |
| Vancomycin | 32 | 32 | 9.306 | 0.5-32 |
| Teicoplanin | 2 | 32 | 2.04 | 0.03-32 |
| Synercid | 1 | 16 | 1.614 | 0.062-16 |
| Amox | 1 | 256 | 1.366 | 0.062-256 |
| Amox + clav | 0.25 | 8 | 0.423 | 0.062-32 |
| Imipenem | 0.064 | 1 | 0.084 | 0.008-4 |
| Clindamycin | 0.125 | 8 | 0.208 | 0.008-64 |
| Metronidazole | 0.5 | 2 | 0.48 | 0.062-32 |
| Gatifloxacin | 0.5 | 2 | 0.659 | 0.06-32 |
| Moxifloxacin | 0.5 | 2 | 0.566 | 0.03-32 |

Some of the MICs obtained are as follows:

| | Com- | | | | | | | | | | | | |
|---|---|---|---|---|---|---|---|---|---|---|---|---|---|
| | pound | | | | Quin/ | | | | | | | | |
| Organism | No. 16 | Linezolid | Vanco | Teico | dal | Amox | Ax/clav | Imipen | Clinda | Metron | Gati | Moxi | Cefinase |
| Clostridium camis | 0.03 | 2 | 2 | <=.06 | 0.5 | <=.125 | <=.125 | 0.06 | 0.03 | <=.125 | 0.25 | 0.25 | − |
| Clostridium camis | 0.016 | 2 | 2 | <=.06 | 0.5 | <=.125 | <=.125 | 0.06 | 0.03 | <=.125 | 0.25 | 0.25 | − |
| Clostridium | 0.03 | 2 | 0.5 | <=.06 | 0.5 | <=.125 | <=.125 | 0.06 | 1 | 1 | 1 | 0.5 | − |
| perfringens | | | | | | | | | | | | | |
| Clostridium | 0.03 | 2 | 0.5 | <=.06 | 0.5 | <=.125 | <=.125 | 0.25 | 0.5 | 1 | 1 | 0.5 | − |
| perfringens | | | | | | | | | | | | | |
| Clostridium difficile | 0.03 | 2 | 2 | 0.25 | 0.5 | 1 | 1 | 4 | 2 | 0.25 | 1 | 1 | − |
| Clostridium difficile | 0.03 | 2 | 4 | 0.25 | 0.5 | 2 | 1 | 4 | 4 | 0.25 | 2 | 2 | − |
| Bacteroides fragilis | 0.03 | 4 | >16 | >16 | 8 | 32 | 0.5 | 0.06 | 0.5 | 0.5 | 1 | 0.25 | + |
| Bacteroides fragilis | 0.06 | 4 | >16 | >16 | >8 | >128 | 4 | 0.25 | 2 | 1 | 1 | 0.5 | + |
| Bacteroides fragilis | 0.06 | 4 | >16 | >16 | >8 | >128 | 8 | 0.5 | 1 | 1 | 1 | 0.5 | + |
| Preotella | 0.125 | 4 | >16 | 16 | >8 | >128 | 32 | 0.5 | 8 | 0.5 | 1 | 0.25 | + |
| (Bacteroides) | | | | | | | | | | | | | |
| disiens | | | | | | | | | | | | | |
| Prevotella | 0.06 | 4 | >16 | >16 | 8 | >128 | 8 | 0.03 | 4 | 1 | 1 | 0.5 | + |
| (Bacteroides) | | | | | | | | | | | | | |
| disiens | | | | | | | | | | | | | |
| Prevotella bivia | 0.125 | 1 | >16 | 1 | 2 | <=.125 | <=.125 | 0.03 | >32 | 1 | 2 | 2 | − |
| Prevotella | 0.016 | 0.5 | >16 | 0.5 | 0.25 | 4 | <=.125 | <=.016 | <=.016 | 0.5 | 0.25 | 0.5 | + |
| intermedia | | | | | | | | | | | | | |
| Prevotella | 0.016 | 1 | >16 | 0.5 | 0.25 | <=.125 | <=.125 | <=.016 | <=.016 | 0.25 | 0.25 | 0.5 | − |
| intermedia | | | | | | | | | | | | | |
| Prevotella | 0.06 | 1 | >16 | 2 | 1 | <=.125 | <=.125 | <=.016 | <=.016 | 0.25 | 0.5 | 1 | − |
| melaninogenica | | | | | | | | | | | | | |
| Prevotella | 0.125 | 2 | >16 | 4 | 2 | 64 | 2 | 0.03 | 0.03 | 0.5 | 8 | 16 | + |
| melaninogenica | | | | | | | | | | | | | |
| Porphyromonas | <=.008 | 1 | 2 | 0.125 | <=.125 | <=.125 | <=.125 | 0.03 | <=.016 | <=.125 | 0.25 | 0.5 | − |
| asaccharolytica | | | | | | | | | | | | | |
| Fusobacterium | 0.03 | 0.25 | >16 | >16 | 8 | 128 | 8 | 0.25 | 0.06 | <=.125 | 0.25 | 0.25 | + |
| montiferum | | | | | | | | | | | | | |
| Fusobacterium | 0.03 | 0.25 | >16 | >16 | >8 | >128 | 32 | 0.5 | 0.125 | <=.125 | 0.25 | 0.25 | + |
| montiferum | | | | | | | | | | | | | |
| Fusobacterium | 0.03 | 0.25 | >16 | >16 | >8 | 1 | 1 | 1 | 0.06 | <=.125 | 0.25 | 0.5 | − |
| montiferum | | | | | | | | | | | | | |
| Fusobacterium | 0.03 | 0.25 | >16 | >16 | 4 | 1 | 1 | 1 | 0.06 | <=.125 | 0.5 | 0.5 | − |
| montiferum | | | | | | | | | | | | | |
| Fusobacterium | <=.008 | 0.5 | >16 | >16 | 2 | <=.125 | <=.125 | <=.016 | 0.06 | <=.125 | 0.25 | 0.125 | − |
| nucleatum | | | | | | | | | | | | | |
| Fusobacterium | 0.016 | 0.5 | >16 | >16 | 1 | <=.125 | <=.125 | <=.016 | 0.06 | <=.125 | 0.25 | 0.125 | − |
| nucleatum | | | | | | | | | | | | | |
| Fusobacterium | 0.016 | 0.5 | >16 | >16 | 1 | <=.125 | <=.125 | 0.03 | 0.06 | <=.125 | 0.5 | 0.25 | − |
| nucleatum | | | | | | | | | | | | | |
| Fusobacterium | 0.016 | 1 | >16 | >16 | 4 | <=.125 | <=.125 | <=.016 | 0.125 | 0.5 | 0.5 | 0.25 | − |
| nucleatum | | | | | | | | | | | | | |
| Porphyromonas | <=.008 | 1 | 8 | <=.06 | 0.25 | <=.125 | <=.125 | <=.016 | <=.016 | <=.125 | 0.06 | 0.03 | − |
| gingivalis | | | | | | | | | | | | | |
| Fusobacterium | 1 | 1 | >16 | >16 | >8 | 1 | 1 | 0.5 | 16 | <=.125 | 2 | 2 | − |
| varium | | | | | | | | | | | | | |
| Fusobacterium | 0.25 | 1 | >16 | >16 | >8 | 1 | 1 | 0.5 | 1 | <=.125 | >16 | >16 | − |
| varium | | | | | | | | | | | | | |
| P. acnes | 1 | 0.5 | 0.5 | 0.25 | <=.125 | <=.125 | <=.125 | <=.016 | 0.06 | >16 | 0.25 | 0.25 | − |
| P. acnes | 1 | 0.5 | 1 | 0.25 | <=.125 | <=.125 | <=.125 | <=.016 | 0.06 | >16 | 0.25 | 0.25 | − |
| P. acnes | 1 | 0.5 | 0.5 | 0.25 | <=.125 | <=.125 | <=.125 | <=.016 | 0.06 | >16 | 0.125 | 0.125 | − |
| P. acnes | 1 | 0.5 | 0.5 | 0.25 | <=.125 | 0.25 | 0.25 | 0.03 | 0.06 | >16 | 0.25 | 0.25 | − |
| Peptostreptococcus | <=.008 | 0.5 | 0.5 | 0.125 | <=.125 | 0.25 | 0.25 | 0.125 | 0.03 | 0.5 | 0.25 | 0.125 | − |
| asaccharolyticus | | | | | | | | | | | | | |
| Fusobacterium | 0.5 | 1 | >16 | >16 | >8 | 1 | 1 | 1 | 4 | <=.125 | 4 | 4 | − |
| varium | | | | | | | | | | | | | |
| Peptostreptococcus | <=.008 | 1 | 0.125 | 0.125 | 0.25 | <=.125 | <=.125 | <=.016 | 0.25 | 2 | 1 | 0.25 | − |
| asaccharolyticus | | | | | | | | | | | | | |
| Peptostreptococcus | 0.016 | 2 | 0.5 | 0.125 | 0.25 | 0.25 | 0.25 | 0.06 | 0.125 | 0.5 | 0.125 | 0.06 | − |
| magnum | | | | | | | | | | | | | |
| Peptostreptococcus | <=.008 | 1 | 0.25 | <=.06 | 0.25 | <=.125 | <=.125 | <=.016 | 0.06 | 0.25 | 0.125 | 0.06 | − |
| magnum | | | | | | | | | | | | | |
| Peptostreptococcus | 0.016 | 1 | 0.25 | 0.125 | 0.25 | 0.25 | 0.25 | 0.06 | 0.125 | 1 | 0.5 | 0.25 | − |
| magnum | | | | | | | | | | | | | |
| Peptostreptococcus | <=.008 | 2 | 0.25 | 0.125 | 0.25 | 0.5 | 0.5 | 0.06 | 1 | 0.5 | 0.25 | 0.25 | − |
| magnum | | | | | | | | | | | | | |
| Peptostreptococcus | <=.008 | 0.5 | 1 | 0.125 | 0.5 | <=.125 | <=.125 | 0.03 | 4 | 0.25 | 0.5 | 0.25 | − |
| micros | | | | | | | | | | | | | |
| Peptostreptococcus | 0.016 | 1 | 1 | <=.06 | 1 | <=.125 | <=.125 | 0.03 | 0.25 | 0.5 | 4 | 2 | − |
| micros | | | | | | | | | | | | | |
| Peptostreptococcus | 0.016 | 1 | 1 | <=.06 | 0.5 | <=.125 | <=.125 | 0.03 | 0.125 | 0.5 | 0.5 | 0.5 | − |
| micros | | | | | | | | | | | | | |
| Peptostreptococcus | 0.016 | 0.5 | 1 | 0.125 | 1 | <=.125 | <=.125 | 0.03 | 0.25 | 0.25 | 16 | 16 | − |
| micros | | | | | | | | | | | | | |
| Peptostreptococcus | <=.008 | 0.5 | 1 | 0.125 | 1 | <=.125 | <=.125 | 0.03 | 2 | 1 | 1 | 0.5 | − |
| tetradius | | | | | | | | | | | | | |
| Peptostreptococcus | <=.008 | 0.5 | 1 | <=.06 | 1 | <=.125 | <=.125 | 0.03 | 0.5 | 1 | 0.5 | 0.5 | − |
| tetradius | | | | | | | | | | | | | |
| Peptostreptococcus | 0.016 | 0.5 | 0.125 | 0.25 | 0.25 | <=.125 | <=.125 | <=.016 | 0.25 | 2 | 0.5 | 0.25 | − |
| prevotii | | | | | | | | | | | | | |
| Peptostreptococcus | <=.008 | 0.5 | 0.125 | <=.06 | 0.25 | 0.25 | <=.125 | <=.016 | 0.125 | 1 | 1 | 0.25 | − |
| prevotii | | | | | | | | | | | | | |
| Eubacterium leutum | <=.008 | 1 | 1 | <=.06 | 0.25 | 1 | 1 | 0.25 | 0.06 | 0.25 | 0.25 | 0.5 | − |
| Eubacterium leutum | <=.008 | 1 | 1 | 0.125 | 0.25 | 1 | 1 | 0.5 | 0.25 | 0.25 | 0.5 | 0.5 | − |
| Eubacterium leutum | <=.008 | 1 | 1 | 0.125 | 0.25 | 1 | 1 | 0.5 | 0.25 | 0.5 | 0.5 | 0.5 | − |
| Eubacterium leutum | <=.008 | 1 | 1 | 0.125 | 0.25 | 1 | 1 | 0.5 | 0.06 | 0.5 | 0.5 | 0.5 | − |
| Fusobacterium | <=.008 | 0.5 | >16 | >16 | 0.25 | 0.5 | 0.5 | 0.25 | 0.03 | 0.25 | 0.5 | 1 | − |
| necrogenes | | | | | | | | | | | | | |

Activity Against Catheter Related Infections

During last five decades metals or plastics have been increasingly used for different types of devices. More than 150 million intravascular catheters are purchased annually by clinics and hospitals in USA, including more than 5 million central venous and pulmonary artery catheters leading to at least 400,000 catheters related blood stream infections. These increase the risk of morbidity (such as prolonged hospital stay) and deaths. Mortality rates associated with catheter related blood stream infection range from 10-20%. Food and Drug Administration (FDA Guidance for Industry; October 1999) Of all the problems associated with such implants, the most severe is infections. The commonest microorganism involved in such infections are Staphylococcus aureus and S. epidermidis. Though microorganisms may be implicated. Journal of Antimicrobial Chemotherapy (JAC 1993; 31(SD): 97-102)

At the present time, there are no agents for this indication and the standard regimens includes removal of catheter. Vancomycin is usually recommended in the hospital or countries with an increased incidence of MRSA, because of its activity against coagulase negative staphylococcus and S. aureus. Clinical Infectious Diseases (CID 2001; 32: 1249-1272)

S. epidermidis is the causative agent in many incidents of infection of implanted medical devices such as catheters, pacemakers, prosthetics joints, cardiac valves and central venous system shunts. These infections often recur and tend to be difficult to treat with antibiotics agents. Removal of the devices with concurrent administration of antibiotics is usually the only method of eradicating the focus of infection.

The biofilm mode of growth is recognized as being of prime importance in the establishment and maintenance of bacterial population within a wide variety of natural habitats including colonization and infections of medical devices. This to some extent protects the sessile population from any major fluctuations in the micro environment from host defences and also from therapeutic effects of antibiotics. Resistance of device associated infections has been attributed variously to failure of antibiotics, to penetrate the glycocalyix, show growth rate within nutrient deprived biofilms and/or to innate properties in adherent cells.

In device related infections, the correlation between MIC levels and clinical efficacy is poor, leading to the dogma with infected implants have to be removed in order to achieve cure. The main characteristics of such infections are the microbial adherence effected by the biofilm and the low growth rate of surface adherent microorganisms. The discrepancy between the results of routine antibiotic susceptibility testing and treatment success in device related infections may therefore be due to the fact that bacterial biofilms have different resistant pattern compared with planktonic bacterial. It has been demonstrated that cure rate in experimental device related infections can be predicted by the in vitro bactericidal effect of antibiotics on non-growing and adherent bacteria.

To demonstrate the usefulness of Compound No. 16 in device related infections we have performed two tests of experiments:

1. Inhibition of slime production

2. Activity against glass adherent bacteria.

To study the effect of Compound No. 16 on the inhibition of biofilm production, the following study was carried out. Since Mueller Hinton broth does not support the formation of biofilm, trypticase soy broth with 2% glucose was used to stimulate biofilm formation by MRSA 1029/99 and MRSE 879/247 (both recent clinical isolates collected from tertiary care hospital). Bacterial suspensions (in triplicate) were exposed to doubling dilution of antibiotics and incubated overnight at 37° C. with constant shaking (100 rpm). Next day, after aspirating the medium, biofilm was stained with safranin (0.1%) for 1 hour at room temperature, washed with distilled water, tapped dried and stain extracted into 200 μl of 0.2M NaoH and OD measured at 544 nm. Relative inhibition was determined by using the formula:

% inhibition=100−[(OD of treated well/OD of Reference well)×100]

References:

Blake J E, Metcalfe M A. A shared noncapsuler antigen responsible for false positive reaction by Staphylococcus epidermidis in commercial agglutination test for Staphylococcus aureus. J.Clinical Microbiol. 2001;39:544-550

Polonio R E et al. Eradication of biofilm forming Staphylococcus epidermidis(RP62A) by a combination of Sodium salicylate and Vancomycin. Antimicrobial Agents Chemother. 2001;45:3262-3266

Results:

Formation of Biofilm inhibition occurs at lower a concentration by Compound No. 16 as depicted in the graphs.

[image]

[image]

[image]

Compound No. 16 is Active Against Adherent Bacteria:

Linezolid has been shown to be active against nearly all clinically relevant gram positive pathogens with MIC₉₀ of 2 to 4 μg/ml, while the Cmax is 12 to 16 μg/ml. Since the mechanism of action of Linezolid is novel, it is active against all gram positive bacteria irrespective of their susceptibility to other antibiotics. Though the action is bacteriostatic, it has been very difficult to generate resistant mutants in the laboratory. However, within months of clinical use resistance in Vancomicin Resistant Enterococci (VRE) and and Methicillin Resistant Staphylococcus Aureus (MRSA) has been reported. The common feature in both reports is the presence of foreign body (catheter) in these patients leading to treatment failure and development of resistant mutants.

We investigated the change in MIC of Linezolid, Vancomycin, Synercid and Compound No. 16 in a sintered glass adherent bacteria model with MRSE 879 bacteria and found that though the broth MICs were Linezolid (2 μg/ml), Vancomycin (1 μg/ml), Synercid (0.5 μg/ml) and Compound No. 16 (0.5 μg/ml), the concentration which would kill adherent bacteria were Linezolid (32 μg/ml), Vancomycin (8 μg/ml), Synercid (2 μg/ml) and Compound No. 16(2 μg/ml).

[image]

Agar Dilution Method for M. tuberculosis:

Antibiotics were incorporated at concentrations of 8, 4, 2, 1, 0.5, 0.25, 0.125, 0.06 and 0.03 μg/ml into plate of Middlebrook 7H10 agar medium supplemented with OADC enrichment (Difco) Test organisms were grown in 7H9 medium (Difco) containing 0.05% Tween 80. After 7 days of incubation at 37° C. the brutish were adjusted to 1 MacFarland, the organisms were then diluted 10 fold in sterile water containing 0.05% of Tween 80. The resulting bacterial suspensions were spotted on to the predried supplemented 7H10 plates. After 21 days of incubation at 37° C. the MICs were recorded as the lowest concentration of the drug that completely inhibited the growth of the organism.

MIC (μg/ml)

| | | | |
|---|---|---|---|
| Drugs | MIC₅₀ | MIC₉₀ | G.M. |

Mycobacterium tuberculosis

| | | | |
|---|---|---|---|
| Rifampicin | 64 | 64 | 6.35 |
| Isoniazid | 8 | 64 | 3.17 |
| Sparfloxacin | 1 | 2 | 0.53 |
| Clarithromycin | 16 | 32 | 12.69 |
| Linezolid | 8 | 64 | 8 |
| Compound No. 16 | 4 | 64 | 5.44 |

Mycobacterium avium intracellulare

| | | | |
|---|---|---|---|
| Rifampicin | 1 | 32 | 1.999 |
| Isoniazid | 32 | 64 | 18.149 |
| Sparfloxacin | 4 | 8 | 3.526 |
| Clarithromycin | 1 | 4 | 1.554 |
| Linezolid | 16 | 64 | 20.587 |
| Compound No. 16 | 8 | 32 | 8.52 |

The compounds of the present invention represented by general Formula I may be prepared by the method of reaction in Scheme I. Key intermediate amines of Formula V for the analogue preparation were prepared by the synthetic procedures described below from commercially available reagents. The compounds of Formula I were made by either Method A, B, or C.

Amines already known in the literature are given by reference and if they have been made by a different procedures they are described in detail.

Mainly five different amines of Formula V identified as five different cores namely

(S)-N-[[3-[3-Fluoro-4-(N-piperazinyl)phenyl]-2-oxo-5-oxazolidinyl]methyl]acetamide (core I),

(S)-N-[[3-[3-Fluoro[4-[3-(1α,5α,6α)-6-[N-methyl]amino]-3-azabicyclo-[3.1.0]hexane]benzyl]-2-oxo-5-oxazolidinyl]methyl]acetamide (core II),

(S)-N-[[3-[3-Fluoro[4-[3-(1α,5α,6α)-6-[N-(5-nitro-2-furylmethyl)-N-methyl]amino]-3-azabicyclo-[3.1.0]hexane]phenyl]-2-oxo-5-oxazolidinyl]methyl]-acetamide (core III),

(S)-N-{3-[4-[4-N-methylaminopeperidin-1-yl]-3-fluorophenyl)-2-oxo-oxazolidin-5-yl]methyl acetamide (core IV), and

(S)-N-[[3-[3[Fluoro-4-(N-1-homopiperazinyl)phenyl]-2-oxo-5-oxazolidinyl]methyl]acetamide (core V)

are shown in the examples given below.

Most of the compounds were characterized using NMR, IR and were purified by chromatography. Crude products were subjected to column chromatographic purification using silica gel (100-200 or 60-120 mesh) as stationery phase.

The examples mentioned below demonstrate the general synthetic procedure as well as the specific preparation for the preparation for the preferred compound. The examples are given to illustrate the details of the invention and should not be constrained to limit the scope of the present invention.

### EXAMPLE 1

### Analogues of (S)-N-[[3-[3-Fluoro-4-(N-piperazinyl)phenyl]-2-oxo-5-oxazolidinyl]methyl]acetamide (Core I)

The heteroaromatic group with the corresponding appendage can be introduced on the nitrogen atom of ring C of compounds of Formula I by one of the methods described below:

Method-A:

General Procedure:

Amine of structure of Formula V is reacted with a heteroaromatic compounds of Formula VI having corresponding R₁₂ appendages such as —CH₂R₁₃, —COR₁₃ or —CH(CH₃)R₁₃ wherein R₁₃ is a suitable leaving group well known to one of ordinary skill in the art such as fluoro, chloro, bromo, SCH₃, —SO₂CH₃, —SO₂CF₃ or OC₆H₅ etc.

The reaction is done in a suitable solvent such as dimethylformamide, dimethylacetamide, ethanol or ethylene glycol at a suitable temperature in the range of −78° C. to 180° C. to afford compounds of Formula II. The presence of a suitable base such as triethylamine, diisopropyl amine, potassium carbonate, sodium bicarbonate is useful in some cases to improve the yield of the reaction.

The following compounds were made following this method:

Compound No. 01 (S)-N-[[3-[3-Fluoro-4-[N-1-[4-(2-furoyl)piperazinyl]]-phenyl]-2-oxo-5-oxazolidinyl]methyl]acetamide

(S)-N-[[3-[3-Fluoro-4-(N-piperazinyl)phenyl]-2-oxo-5-oxazolidinyl]methyl]acetamide prepared by the method given in U.S. Pat. No. 5,700,799 (1.2 g, 3.57 mmol) was dissolved in dry dimethyl formamide (35 ml). To this was added K₂CO₃ (2.47 g; 17.87 mmol) and furoyl chloride (0.56 g, 10.68 mmol). The reaction mixture was stirred at 25° C. for 5.0 hr. TLC of the reaction mixture was monitored. A faster moving spot was observed. Solvent was removed and the residue was dissolved in dichloromethane, washed with water, dried over sodium sulphate, and solvent was removed. The residue was digested with ether and filtered to yield 800 mg of white crystalline solid 225.5-226.5° C.

δppm (CDCl₃): 7.50-7.44 (m, 2H), 7.09-7.06 (m, 2H), 6.95-6.89 (m, 1H) 6.50 (bs, 1H) 4.76 (bs, 1H), 4.05-3.19 (m, 9H), 3.09 (bs, 4H), 2.02 (s, 3H).

Compound No. 02: (S)-N-[[3-[3-Fluoro-4-[N-1[4-{2-furyl(5-formyl)methyl}]piperazinyl]phenyl]-2-oxo-5-oxazolidinyl]methyl]acetamide

The title compound was made with (S)-N-[[3-[3[Fluoro-4-(N-1-piperazinyl)phenyl]-2-oxo-5-oxazolidinyl]methyl acetamide and 5-chloromethyl 2-furfuraldehyde using Method A.

Compound No. 03: (S)-N-[[3-Fluoro-4-[N-1[4-(2-furyl-(5-carboxyethyl)methyl)-piperazinyl]phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide ethyl-5-(chloromethyl)-2-furan-carboxylate using Method A.

Compound No. 04: (S)-N-[[3-Fluoro-4-[N-1[4-(5-bromo-2-furoyl)]piperazinyl]-phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-bromo-2-furoyl chloride using Method A.

Compound No. 05: (S)-N-[[3-Fluoro-4-[N-1[4-(5-chloromethyl-2-furoyl)piperazinyl]phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-chloromethyl-2-furoyl chloride using Method A.

Compound No. 06: (S)-N-[[3-Fluoro-4-[N-1[4-(5-nitro-2-furoyl)piperazinyl]phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-nitro-2-furoyl chloride using Method A.

Compound No. 07: (S)-N[[3-[3-Fluoro-4-[N-1[4-{2-(2-thienyl)dicarbonyl}]-piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 2-thiophenglyoxylyl chloride using Method A.

δppm (CDCl₃): 7.84(m, 2H, Ar—H), 7.47(dd, 1H, Ar—H), 7.2(m, 1H, Ar—H), 7.07(d, 1H, Ar—H), 6.92(t, 1H, Ar—H), 5.98(t, 1H, NH), 4.76(m, 1H, CH), 4.0(t, 1H, CH), 3.5-3.95 (m, 7H, CH₂), 3.15 (m, 2H, CH₂), 3.06 (m, 2H Cl₂), 2.02 (s, 3H, CH₃)

Compound No. 08: (S)-N[[3-[3-Fluoro-4-[N-1[4-(3-furoyl)]piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl]acetamide and 3-furoyl chloride using Method A.

δppm (CDCl₃): 8.06(s, 1H, Ar—H), 7.49(m, 2H, Ar—H), 7.09(d, 1H, Ar—H), 6.76(t, 1H, Ar—H), 6.57 (s, 1H, Ar—H), 6.03(br s, 1H, NH), 4.77 (m, 1H, CH), 4.2-3.5(m, 8H, CH₂), 3.06(m, 4H, CH₂), 2.02(s, 3H, CH₃)

Compound No. 09: (S)-N[[3-[3-Fluoro-4-[N-1[4-{2-furyl(5-bromo)methyl}]piperaziny]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-bromo-2-chloromethylfuran using Method A.

δppm (CDCl₃): 7.47 (d, 1H, Ar—H), 7.06 (d, 1H, Ar—H), 6.91 (t, 1H, Ar—H), 6.47 (d, 1H, Ar—H), 6.32 (d,1H, Ar—H), 5.98 (t, 1H, NH), 4.76 (m, 1H, CH), 4.02 (t, 1H, CH), 3.4-3.85 (m, 9H, CH₂), 3.07 (m, 4H, CH₂), 2.02 (s, 3H, CH₃).

Compound No. 10: (S)-N[[3-[3-Fluoro-4-[N-1[4-{2-thienyl(5-chloro)methyl}]-piperazinyl]phenyl]2-oxo-5-oxazolidinyl]methyl]acetamide

The title compound was made with (S)-N-[[3-[3[Fluoro-4-(N-1-piperazinyl)phenyl]-2-oxo-5-oxazolidinyl]methyl acetamide and 5-chloro-2-chloromethylthiophene using Method A.

δppm (CDCl₃): 7.42 (dd, 1H, Ar—H), 7.05 (dd, 1H, Ar—H), 6.92 (t, 1H, Ar—H), 6.74 (d, 2H, Ar—H), 6.00 (m,1H, CH), 4.74 (m, 1H, CH), 4.01 (t, 1H, CH), 3.3-3.8 (m, 5H, CH₂), 3.08 (m, 4H, CH₂), 2.66 (m 4H, CH₂) 2.01 (s, 3H, CH₃).

Compound No. 11: (S)-N[[3-[3-Fluoro-4-[N-1[4-(2-furylmethyl)]piperazinyl]-phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 2-chloromethylfuran using Method A.

δppm (CDCl₃): 7.49 (m, 2H, Ar—H), 7.07 (d, 1H, Ar—H), 6.91 (t, 1H, Ar—H), 6.51 (d, 1H, Ar—H), 6.4 (d,1H, Ar—H), 6.1 (t, 1H, NH), 4.75 (m, 1H, CH), 4.1-3.25 (m,10H, CH₂), 3.06 (m, 4H, CH₂), 2.03 (s, 3H, CH₃).

Compound No. 12: (S)-N-[[3-[3-Fluoro-4-[N-1[4-(2-thienylmethyl)]piperazinyl]-phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 2-chloromethylthiophen using Method A.

δppm (CDCl₃): 7.4 (m, 1H, Ar—H), 6.94 (m, 5H, Ar—H), 6.08 (t, 1H, NH), 4.71 (m, 1H, CH), 4.1-3.4 (m, 6H, CH₂), 3.08 (m, 4H, CH₂), 2.73 (m, 4H, CH₂), 1.98 (s, 3H, CH₃).

Compound No. 13: (S)-N[[3-[3-Fluoro-4-[N-1[4-(2-thienylacetyl)]piperazinyl]-phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 2-thiophenacetyl chloride using Method A.

δppm (CDCl₃): 7.45 (dd, 1H, Ar—H), 7.23 (d, 1H, Ar—H), 7.07 (d, 1H, Ar—H), 6.96 (m, 3H, Ar—H), 6.05 (t,1H, CH), 4.7 (m, 1H, CH), 2.75-4.1 (m, 10H, CH₂), 3.01 (m, 4H, CH₂), 2.03 (s, 3H, CH₃).

Compound No. 14: (S)-N-[[3-[3-Fluoro-4-[N-1[4-{2-thienyl(4-bromo)methyl}]-piperazinyl]phenyl]-2-oxo-5-oxazolidinyl]methyl]acetamide

The title compound was made with (S)-N-[[3-[3[Fluoro-4-(N-1-piperazinyl)phenyl]-2-oxo-5-oxazolidinyl]methyl acetamide and 4-bromo-2-chloromethylthiophen using Method A.

δppm (CDCl₃): 7.44 (dd, 1H, Ar—H), 7.2-6.8 (m, 4H, Ar—H), 5.98 (t, 1H, Ar—H), 4.76 (m, 1H, CH), 4.02 (t, 1H, CH), 3.85-3.35 (m, 5H, CH₂), 3.1 (m, 4H, CH₂), 2.69 (m, 4H, CH₂), 2.03 (s, 3H, CH₃).

Method B:

Compound No. 15: (S)-N-[[3-[3-fluoro-4-[N-1-[4-{2-furyl-(5-nitro)methyl}]piperazinyl]phenyl]-2-oxo-5-oxazolidinyl]methyl]acetamide

To a suspension of (S)-N-[[3-[3[Fluoro-4-(N-1-piperazinyl)phenyl]-2-oxo-5-oxazolidinyl]methyl acetamide (770 mg, 2.29 mmol) in dichloromethane or THF (40 ml) in a round bottom flask (100 ml) filled with guard tube, was added molecular sieves (4A) followed by 5-nitro-2-furfural (420 mg, 2.98 mmol). The reaction mixture was stirred at 25° C. for 1.5 hr. Sodium triacetoxy borohydride (1.93 g, 9.10 mmol) was then added to the reaction mixture. The whole reaction mixture was allowed to stir overnight at 25° C. TLC of the reaction mixture showed a faster moving spot compared to piperazine derivative. The reaction mixture was filtered through a Buckner funnel. It was washed with dichloromethane. Organic layer was washed with water, dried over sodium sulphate and solvent was removed to give crude product which was then purified by silica gel column using 2% methanol in chloroform as eluent to afford the title compound 417 mg of m.p. 133-135° C. (IPA).

δppm (CDCl₃): 7.48 (d, 1H), 7.34 (m, 1H), 7.12 (d, 1H), 6.98 (t, 1H), 6.56 (d, 1H), 6.07 (bs, 1H), 4.81 (m, 1H), 4.07 (t, 1H), 3.69-3.53 (m, 5H) 3.16 (bs, 4H), 2.78 (bs, 4H), 2.07 (s, 3H).

Compound No. 16: Hydrochloric Salt of (S)-N-[[3-Fluoro-4-[N-1[4-{2-furyl(5-nitro)methyl}]piperazinyl]phenyl]-2-oxo-5-oxazolidinyl]methyl]acetamide

(S)-N-[[3-Fluoro-4-[N-1[4-{2-furyl-(5-nitro)methyl}]piperazinyl]-phenyl]-2-oxo-5-oxazolidinyl]-methyl]acetamide hydrochloride.

To an ethanolic solution of (S)-N-[[3-Fluoro-4-[N-1[4-{2-furyl-(5-nitro)-methyl}]piperazinyl]phenyl]-2-oxo-5-oxazlidinyl]methyl]acetamide (365 mg, 0.75 mmol in 7 ml of absolute ethanol) was added 0.30 ml of HCl in ethanol (2.6 N, 0.75 mmol) in cold (5° C.) condition. The whole reaction mixture was stirred at 5-10° C. for 2.0 hr. No change in TLC was observed.

Solvent was removed. The residue was digested with dichloromethane and the solid was crystallized from methanol isopropyl alcohol mixture to give the desired compound in 111 mg of 97% pure by HPLC. Mass: 461.8 (M+H⁺), 483.9 (M+Na⁺)

Compound No. 17: Citrate Salt of (S)-N-[[3-Fluoro-4-[N-1[4-{2-furyl(5-nitro)-methyl}]piperazinyl]phenyl]-2-oxo-5-oxazolidinyl]methyl]acetamide

Citrate salt of Compound No. 15 was made according to the method described for Compound No. 16 by using citric acid in molar proportions.

Compound No. 18: (S)-N[[3-[3-Fluoro-4-[N-1[4-(2-pyrrolylmethyl)]piperazinyl]-phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 2-pyrrolecarboxaldehyde using Method B.

δppm (CDCl₃): 8.76(br s, 1H, NH), 7.38(d, 1H, Ar—H), 7.04(d, 1H, Ar—H), 6.91(t, 1H, Ar—H), 6.77(s, 1H, Ar—H), 6.11(m, 3H, Ar—H, NH), 4.75 (m, 1H, CH), 4.0(t,1H, CH), 3.8-3.5(m, 5H, CH₂), 3.08(m, 4H, CH₂), 2.65(m, 4H, CH₂), 2.01(s, 3H, CH₃)

Compound No. 19: (S)-N[[3-[3-Fluoro-4-[N-1[4-{2-thienyl(3-methyl)methyl}]-piperazinyl]phenyl]2-oxo-5-oxazolidinyl]methyl]acetamide

The title compound was made with (S)-N-[[3-[3[Fluoro-4-(N-1-piperazinyl)phenyl]-2-oxo-5-oxazolidinyl]methyl acetamide and 3-methyl-2-thiophencarboxaldehyde using Method B.

δppm (CDCl₃): 7.4(d, 1H, Ar—H), 7.15(d,1H, Ar—H), 7.03(d, 1H, Ar—H), 6.92(t, 1H, Ar—H), 6.79(d, 1H, Ar—H), 6.07(t, 1H, NH), 4.75(m, 1H, CH), 3.98(t, 1H, CH), 3.55-3.95(m, 6H, CH₂), 3.09(m, 4H, CH₂), 2.69(m, 3H, CH₂), 2.22(s, 3H, CH₃), 2.01(s, 3H, CH₃)

Compound No. 20: (S)-N[[3-[3-Fluoro-4-[N-1[4-(3-furylmethyl)]piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 3-furaldehyde using Method B.

δppm (CDCl₃): 7.42(m, 3H, Ar—H), 7.04(d, 1H, Ar—H), 6.92(t, 1H, Ar—H), 6.43(s, 1H, Ar—H), 6.0(t, 1H, NH), 4.75(m, 1H, CH), 4.01(t, 1H, CH), 3.8-3.5(m, 3H, CH₂), 3.47(s, 2H, CH₂), 3.1(m, 4H, CH₂), 2.66 (m, 4H, CH₂), 2.01(s, 3H, CH₃)

Compound No. 21: (S)-N[[3-[3-Fluoro-4-[N-1[4-{2-thienyl(5-methyl)methyl}]-piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-methyl-2-thiophencarboxaldehyde using Method B.

δppm (CDCl₃): 7.4(dd, 1H, Ar—H), 7.03(d, 1H, Ar—H), 6.92(t, 1H, Ar—H), 6.71(d, 1H, Ar—H), 6.58(d, 1H, Ar—H), 6.08(t, 1H, NH), 4.75(m, 1H, CH), 3.98(t, 1H, CH), 3.8-3.5(m, 5H, CH₂), 3.07(m, 4H, CH₂), 2.65(m, 4H, CH₂), 2.45(s, 3H, CH₃), 2.01(s, 3H, CH₃)

Compound No. 22: (S)-N[[3-[3-Fluoro-4-[N-1[4-{2-pyrrole(1-methyl)methyl}]-piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and N-methyl-2-pyrrolecarboxaldehyde using Method B.

δppm (CDCl₃): 7.36(d, 1H, Ar—H), 7.04(d, 1H, Ar—H), 6.9(t, 1H, Ar—H), 6.6(s, 1H, Ar—H), 6.02(s, 3H, Ar—H, NH), 4.73(m, 1H, CH), 4.0(t, 1H, CH), 3.8-3.5(m, 6H, CH₂), 3.49(s, 2H, CH₂), 3.02(m, 4H, CH₂), 2.58(m, 4H, CH₂). 2.01(s, 3H, CH₃)

Compound No. 23: (S)-N[[3-[3-Fluoro-4-[N-1[4-{2-thienyl(5-nitro)methyl}]piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-nitro-2-thiophencarboxaldehyde using Method B.

δppm (CDCl₃): 7.80 (d, 1H, Ar—H), 7.45 (dd, 1H, Ar—H), 7.05 (d, 1H, Ar—H), 6.91 (m, 2H, Ar—H), 6.07 (t,1H, NH), 4.76 (m, 1H, CH), 4.2-3.5 (m, 6H, CH₂), 3.11 (m, 4H, CH₂), 2.73 (m, 4H, CH₂), 2.02 (s, 3H, CH₃).

Compound No. 24: (S)-N[[3-[3-Fluoro-4-[N-1[4-[2-furyl{5-(N-thiomorpholinyl)-methyl}methyl]piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-(N-thiomorpholinymethyl)-2-furan-carboxaldehyde using Method B.

δppm (CDCl₃): 7.45 (d, 1H, Ar—H), 7.05 (d, 1H, Ar—H), 6.9 (t, 1H, Ar—H), 6.18 (d, 2H, Ar—H), 6.09 (m,1H, NH), 4.76 (m, 1H, CH), 4.02 (t, 1H, CH), 3.35-3.9 (m, 7H, CH₂), 3.12 (m, 4H, CH₂), 2.75 (m, 11H, CH₂), 2.02 (s, 3H, CH₃).

Compound No. 25: (S)-N[[3-[3-Fluoro-4-[N-1[4-[2-furyl{5-(N-morpholinyl)-methyl}methyl]]piperazinyl]phenyl]2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-(N-morpholinylmethyl)2-furancarboxaldehyde using Method B.

δppm (CDCl₃): 7.5-6.3 (m, 3H, Ar—H), 6.19 (d, 2H, Ar—H), 5.9 (m, 1H, NH), 4.7 (m, 1H, CH), 4.00 (t,1H, CH), 3.3-3.8 (m, 10H, CH₂), 3.09 (m, 4H, CH₂), 2.69 (m, 4H, CH₂), 2.49 (m, 4H, CH₂), 2.01 (s, 3H, CH₃).

Compound No. 26: (S)-N-[[3-Fluoro-4-[N-1[4-{2-furyl(5-acetoxymethyl)-methyl}]piperazinyl]phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-(N-morpholinylmethyl)2-furylcarboxaldehyde using Method B.

δppm (CDCl₃): 7.42 (dd, 1H), 7.06 (dd, 1H), 6.95 (d, 1H), 6.35 (d, 1H), 6.22 (d s, 2H), 5.04 (s, 2H ), 4.02 (bs, 4H, CH₂), 3.74 (t, 1 H), 3.75-3.6 (m, 3H), 3.64 (s, 3H) 3.10 (bs, 4 H) 2.70 (bs, 4H ), 2.06 (s, 3H), 2.02 (S, 3H).

Compound No. 27: (S)-N-[[3-Fluoro-4-[N-1[4-{2-thienyl(5-bromo)methyl}]piperazinyl]phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl acetamide and 5-acetoxy methyl-2-furan-carboxaldehyde by using Method A.

δppm (CDCl₃): 7.42 (dd, 1H, Ar—H), 7.04 (d, 1H, Ar—H), 6.88 (m, 2H, Ar—H), 6.69 (d, 1H, Ar—H), 6.00 (t,1H, NH), 4.76 (m, 1H, CH), 4.01 (t, 1H, CH), 3.4-3.8 (m, 5H, CH₂), 3.07 (m, 4H, CH₂), 2.67 (m, 4H, CH₂).

Compound No. 28: (S)-N-[[3-Fluoro-4-[N-1[4-(5-nitro-2-furylmethyl)piperazinyl]phenyl]-2-oxo-5-oxazolidinyl]methyl]dichloroacetamide

δppm (CDCl₃): 7.41-6.51(m, 6H), 5.96(s, 1H), 4.81(m, 1H), 4.06(t, 1H), 3.77-3.66(m, 5H), 3.11-2.71(m, 8H)

Method C:

Compound No. 29: (S)-N[[3-[3-Fluoro-4-[N-1[4-(5-nitro-2-thienoyl)]piperazinyl]-phenyl]2-oxo-5-oxazolidinyl]methyl]acetamide Hydrochloride

To (S)-N-[[3-[3[Fluoro-4-(N-1-piperazinyl)phenyl]-2-oxo-5-oxazolidinyl]-methyl]acetamide (1.14 mmol) in DMF (10 mL) cooled to 5° C., 5-nitro-2-thienoic acid (0.16 g, 0.95 mmol), N-methylmorpholine (0.12 g, 1.14 mmol) and 1-hydroxybenzotriazole (0.17 g, 1 mmol) were added and the reaction mixture was stirred for 15 min. To it, 1-(3-dimethylaminopropyl)-3-ethylcarbodiimide hydrochloride (0.18 g, 0.95 mmol) was added and the reaction mixture was stirred for 18 hrs allowing it to warm to room temperature. Then the reaction mixture was diluted with 25 mL water and extracted with EtOAc (3×25 mL). The combined organic layers were washed with brine, dried over anhydrous sodium sulfate and evaporated in vacuo. The residue was purified by column chromatography (3% MeOH(CHCl₃) to yield 0.19 g of product. This product was dissolved in dichloromethane (5 mL) and cooled to 5 C. To it 1 mL of satd. ethanolic-HCl solution was added and stirred for 15 min. Then the reaction mixture was evaporated, co-evaporated with ether and dried in vacuo to yied 0.19 g of final product.

δppm (DMSO): 8.2 (t, 1H, Ar—H), 8.1(m, 1H, Ar—H), 7.5(m, 2H, Ar—H), 7.17(d,1H, Ar—H), 7.09(t, 1H, Ar—H), 4.7(m, 1H, CH), 4.08(t,1h, CH), 3.73(m, 6H, CH₂), 3.05 (m, 5H, CH₂), 1.83(s, 3H, CH₃).

Compound No. 30: (S)-N[[3-[3-Fluoro-4-[N-1[4-(2′,2′-diphenyl-2′Hydroxy Acetyl)]piperazinyl]phenyl]-2-oxo-5-oxazolidinyl]methyl]acetamide

The title compound was made with (S)-N-[[3-[3[Fluoro-4-(N-1-piperazinyl)-phenyl]-2-oxo-5-oxazolidinyl]methyl]acetamide and 2,2-diphenyl-2-hydroxy acetic acid using Method C.

### EXAMPLE 2

Analogues of (S)-N-[[3-[3-Fluoro[4-[3-(1α,5α,6α)-6-[N-methyl]amino]-3-azabicyclo[3.1.0]hexane]phenyl]-2-oxo-5-oxazolidinyl]methyl]acetamide(Core II)

The hetero aromatic group with the corresponding appendage can be introduced on the nitrogen atom of ring C of compounds of Formula I by one of the methods described below:

Method A:

General procedure was same as described earlier (method A ). Only the core amine of Formula V is (S)-N-[[3-[3-Fluoro[4-[3-(1α,5α,6α)-6-[N-methyl]amino]-3-azabicyclo[3.1.0]hexane]phenyl]-2-oxo-5-oxazolidinyl]methyl]acetamide here.

Compound No. 31: (S)-N-[[3-[3-Fluoro[4-[3-(1α,5α,6α)-6-[N-(5-nitro-2-furoyl)-N-methyl]amino]-3-azabicyclo-[3.1.0]hexane]phenyl]-2-oxo-5-oxazolidinyl]-methyl]acetamide

Preparation of (S)-N-[[3-[3-Fluoro[4-[3-(1α,5α,6α)-6-[N-methyl]amino]-3-azabicyclo[3.1.0]hexane]phenyl]-2-oxo-5-oxazolidinyl]methyl]acetamide (Core II)

(a) Preparation of 3-Fluoro[4-[3-(1α,5α,6α)-6-[N-(tert butoxy carbonyl)amino]-3-azabicyclo-[3.1.0]hexane]nitrobenzene.

(1α,5α,6α)-6-Amino-3-azabicyclo [3.1.0] hexane (7.0 g, 0.03535 mol) was taken in CH₃CN (50 mL) and diisopropyl ethyl amine (4.5606 g, 0.03535 mol) was added followed by 1,2-difluoro-4-nitrobenzene (5.6212 g, 0.03535 mol) and heated at 70° C. for 4 hrs. The reaction was monitored by the disappearance of the starting material on the TLC (eluent CHCl₃:MeOH (19:1)). The reaction mixture was concentrated under vacuum, triturated with H₂O, filtered, washed with hexane and dried to obtain the title compound. Yield: 10 g

δppm (CDCl₃): 7.94-6.50 (m, 3H), 4.80 (5, 1H) 3.95-3.63 (m, 4H), 2.43 (s, 1H), 1.92 (s, 2H), 1.47 (s, 9H).

(b) Preparation of 3-Fluoro[4-[3-(1α,5α,6α)-6-[N-(tert butoxy carbonyl)-N-methyl]-amino]-3-azabicyclo-[3.1.0]hexane]nitrobenzene

3-Fluoro[4-[3-(1α,5α,6α)-6-[N-(tert butoxy carbonyl)-amino]-3-azabicyclo-[3.1.0]hexane]nitro benzene (10 g, 0.029 mol) was taken in 60 ml THF at 0° C. Sodium hydride (1.06 g, 0.045 mol) was added portion-wise over 5 min. After complete addition the reaction mixture was stirred for 30 min. at 0° C. Methyl iodide (8.42 g, 0.059 mol) was then added over 10 min. at 0° C. followed by tert n-butyl ammonium iodide (1 g). The reaction mixture was stirred for 4 hrs. The reaction mixture was then concentrated under vacuum. H₂O (50 mL) was added followed by extraction with dichloromethane (3×50 mL). The combined organic layer was dried over Na₂SO₄, filtered and concentrated to obtain the title compound. Yield: 10.25 g

δppm (MeOD): 7.91-6.47 (m, 3H), 3.89-3.61 (m, 4H) 2.8 (s, 3H), 2.34 (s, 1H), 1.96 (s, 2H), 1.46 (5, 9H).

(c) Preparation of 3-Fluoro[4-{3-(1α,5α,6α)-6-[N-(tert butoxy carbonyl)-N-methyl amino}-3-azabicyclo-[3.1.0]hexane]aniline.

3-Fluoro[4-[3-(1α,5α,6α)-6-[N-(tert butoxy carbonyl)-N-methyl]-amino]-3-azabicyclo-[3.1.0]hexane]nitrobenzene (26 g, 0.074 mol) was taken in 75 mL THF and 75 mL MeOH. 10% Pd/C (dry) (3 g) was added and the reaction mixture was shaken in a Parr hydrogenator at 40 psi for 3 hours. The reaction mixture was filtered through celite bed. The filtrate was concentrated to obtain the title compound. Yield: 21.2 g

δppm (CDCl₃) (MeOD): 6.55-6.33 (m, 3H), 3.54-3.00 (m, 4H) 2.87 (s, 3H), 2.55 (s, 1H), 1.96 (s, 2H) 1.40 (s, 9H).

(d) Preparation of 3-Fluoro[4-[3-(1α,5α,6α)-6-[N-(tert butoxy carbonyl)-N-methyl]amino]-3-azabicyclo-[3.1.0]hexane]benzyloxy carbamate

3-Fluoro[4-[3-(1α,5α,6α)-6-[N-(tert-butoxy carbonyl)-N-methyl)amino]-3-azabicyclo[3.1.0]hexane]aniline (21 g, 0.065 mol) was taken in THF (100 ml and cooled to −15° C. Sodium bicarbonate (27.47 g, 0.32 mol) was added followed by benzyl chloroformate (14.5 g, 0.055 mol) which was added slowly over 30 min. After complete addition the stirring was combined for the maintaining the temperature between 0-5° C. The reaction was monitored by the disappearance of the reaction mixture on TLC (eluent CHCl₃:MeOH:9:1). The reaction mixture was filtered and filtrate concentrated under vacuum. H₂O (20 ml) was added and extracted with CH₂Cl₂ (3×100 ml). The combined organic layer was dried over Na₂SO₄. This was filtered and the filtrate concentrated. The semisolid was triturated with MeOH. The solid was filtered to obtain the title compound.

δppm (CDCl₃):7.4:6.5 (m, 8H), 5.24 (s, 2H), 3.8-3.3(m, 4H), 2.92 (s, 3H), 2.61 (s, 1H), 1.90 (s, 2H), 1.54 (s, 9H, tBu).

(e) Preparation of (S)-N-[3-[3-Fluoro[4-[3-(1α,5α,6α)-6-(N-(tert butoxy carboxy-N-methyl]amino]-3-azabicyclo[3.1.0]hexane]phenyl]-2-oxo-5-oxazolidinyl]methyl alcohol.

3-Fluoro[4-[3-(1α,5α,6α)-6-(N-(tert butoxy carbonyl)-N-methyl]amino]-3-azabicyclo[3.1.0]hexane]benzyloxy carbamate (21 g, 0.04615 mol) was taken in freshly distilled THF (200 mL). The system was thoroughly flushed with N₂. The temperature was then brought down to −78° C. in acetone dry ice. n-BuLi (59.13 mL of 15% solution in hexane, 0.13846 mol) was added over 30 min. maintaining the temperature at −78° C. The stirring was continued for 2.5 hours at −78° C. R(−) Glycidyl butyrate was added in one go and stirred at −78° C. for further 1.5 hours. The temperature was gradually increased to room temperature and stirred over night. 20% aqueous solution of NH₄Cl (200 ml) was then added gradually added over 10 min. After 30 min. stirring, the organic layer was separated. The aqueous layer was further extracted with EtOAc (3×75 ml). The combined organic was dried over Na₂SO₄, filtered and concentrated. The product was purified by silica gel column chromatography (100-200) eluent (2% MeOH:98% CHCl₃) to yield 14 g.

δppm (CDCl₃): 7.35-6.55 (m, 3H), 4.7 (m, 1H), 3.9-3.8 (m, 4H), 3.7-3.2 (m, 4H), 2.8 (s, 3H, N—CH₃), 2.5 (S, 1H), 1.8 (s, 2H), 1.47 (s, 9H).

(f) Preparation of (S)-N-[3-[3-Fluoro[4-[3-(1α,5α,6α)-6-[N-(tert butoxy carbonyl)-N-Methyl]amino]-3-azabicyclo[3.1.0]hexane]phenyl]-2-oxa-5-oxazolidinyl]methyl methanesulfonate.

(S)-N-[3-[3-Fluoro[4-[3-(1α,5α,6α)-6-[N-(tert butoxy carbonyl)-N-methyl]amino]-3-azabicyclo[3.1.0]hexane]phenyl]-2-oxa-5-oxazolidinyl]methyl alcohol (16 g, 0.038 mol) was taken in 50 ml pyridine at 5-10° C. and methane sulphonyl chloride (12.71 g, 0.14 mol) was added over 5 min. The stirring was continued for 4 hours. The progress of the reaction was monitored by the disappearance of the starting material on TLC (eluent 10% CHCl₃: 10% MeOH). The reaction mixture was filtered, filtrates concentrated under vacuum, washed with H₂O (50 ml) and extracted with CH₂Cl₂ (3×75 mL). The combined organic layer was dried over Na₂SO₄, filtered and filtrate concentrated. This was dried thoroughly under vacuum.

(g) Preparation of (S)-N-[3-[3-Fluoro[4-[3-(1α,5α,6α)-6-[N-(tert butoxy carbonyl)-N-Methyl]amino]-3-azabicyclo[3.1.0]hexane]phenyl]-2-oxa-5-oxazolidinyl]methyl azide.

(S)-N-[3-[3-Fluoro[4-[3-(1α,5α,6α)-6-[N-(tert butoxy carbonyl)-N-methyl]amino]-3-azabicyclo[3.1.0]hexane]phenyl]-2-oxa-5-oxazolidinyl]methyl methane sulphonate (15 g, 0.03 mol) was taken in DMF (50 ml) and NaN₃ (9.76 g, 0.15 mol) was added and heated at 70° C. for 4 hours. The progress of the reaction was monitored by the disappearance of the starting material on TLC. The reaction mixture was filtered. The filtrate was concentrated under vacuum. This was washed with H₂O and extracted EtOAc (3×75 ml). The combined organic layer was dried over Na₂SO₄, filtered and concentrated to obtain the title compound. Yield 11.5 g.

δppm (CDCl₃): 7.3-6.5 (m, 3H), 4.7 (m, 1H)

(h) Preparation of (S)-N-[3-[3-[3-Fluoro[4-[3-(1α,5α,6α)-6-[N-(tert butoxy carbonyl)-N-methyl]amino]-3-azabicyclo[3.1.0]hexane]phenyl]-2-oxo-5-oxazolidinyl]methyl amine

(S)-N-[3-[3-[3-Fluoro[4-[3-(1α,5α,6α)-6-[N-(tert butoxy carbonyl)-N-methyl]amino]-3-azabicyclo[3.1.0]hexane]phenyl]-2-oxo-5-oxazolidinyl]methyl azide (11.3 g, 0.026 mol) was taken in 75 ml MeOH and 75 ml EtOAc and 10% Pd/C was added. The reaction mixture was shaken at 50 psi for 6 hrs. The progress of the reaction was monitored by the disappearance of the starting material on the TLC. The reaction mixture was filtered through a celite bed. The filtrate was concentrated. The product was triturated with diethyl ether. The solid was filtered, to obtain the title compound. Yield −7.6 g.

(i) Preparation of (S)-N-[3-[3-Fluoro[4-[3-(1α,5α,6α)-6-[N-(tert butoxy carbonyl)-N-Methyl]amino]-3-azabicyclo[3.1.0]hexane]phenyl]-2-oxa-5-oxazolidinyl]acetamide.

(S)-N-[3-[3-Fluoro[4-[3-(1α,5α,6α)-6-[N-(tert butoxy carbonyl)-N-methyl]amino]-3-azabicyclo[3.1.0]hexane]phenyl]-2-oxo-5-oxazolidinyl]methyl amine (7.6g, 0.018 mol) was taken in pyridine (8 ml), CH₂Cl₂ (50 mL) and acetic anhydride (2.214 g, 0.0217 mol) at 0-10° C. The reaction mixture was stirred and the progress of the reaction was monitored by the disappearance of the starting material on the TLC eluent (CHCl₃:MeOH:9:1). The reaction mixture was concentrated under vacuum. The concentrate was washed with H₂O (50 mL) and extracted with CH₂Cl₂ (3×50 mL). The combined organic layer was dried over Na₂SO₄, filtered and concentrated. This product was triturated with diethyl ether, filtered and dried to yield the little compound. Yield: 6.6 g.

δppm (CDCl₃): 7.33-6.56 (m, 3H), 6.19 (t, 1H), 4.73 (m, 1H), 3.98 (t, 1H), 3.77-3.2 (m, 7H) 2.8 (s, 3H), 2.52 (s, 1H), 2.0 (s, 3H), 1.96 (S, 2H), 1.48 (s, 9H).

(j) Preparation of (S)-N-[3-[3-Fluoro[4-[3-(1α,5α,6α)-6-[N-Methyl]amino]-3-azabicyclo[3.1.0]hexane]phenyl]-2-oxa-5-oxazolidinyl]acetamide.

(S)-N-[3-[3-Fluoro[4-[3-(1α,5α,6α)-6-[N-(tert butoxy carbonyl)-N-Methyl]amino]-3-azabicyclo[3.1.0]hexane]phenyl]-2-oxa-5-oxazolidinyl]acetamide (1g) was taken in CH₂Cl₂ (50 mL) at 0° C. and CF₃COOH(10 mL) was added and stirred for 4 h. The reaction mixture was concentrated under vacuum. The residue was dissolved in EtOAc and neutralized with solid NaHCO₃. The EtOAc layer was filtered and the filtrate was concentrated to obtain the title compound.

Compound No. 31: (S)-N-[[3-[3-Fluoro[4-[3-(1α,5α,6α)-6-[N-(5-nitro-2-furoyl)-N-methyl]amino]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]amino]-3-azabicyclo[3.1.0]hexane]phenyl]-2-oxa-5-oxazolidinyl]acetamide and 5-nitro-furoyl chloride using Method A.

δppm (CDCl₃): 7.7-60 (m, 6H), 4.74 (m, 1H), 4.0-2.9 (m, 11H), 2.43 (s, 2H), 2.01 (s, 3H), 1.62 (s, 1H), 1.91 (s, 2H)

Compound No. 32: (S)-N-[[3-[3-Fluoro[4-[3-(1α,5α,6α)-6-[N-(3-furoyl)-N-methyl]amino]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-methyl]amino]-3-azabicyclo[3.1.0]hexane]phenyl]-2-oxa-5-oxazolidinyl]acetamide and furan-3-carboxyaldehyde using Method B.

Compound No. 71: (S)-N-[[3-[3-Fluoro[4-[3-(1α,5α,6α)-6-[N-(2-thiopheneacetyl)-N-methyl]amino]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-methyl]amino]-3-azabicyclo[3.1.0]hexane]phenyl]-2-oxa-5-oxazolidinyl]acetamide and 2-thiopheneacetyl chloride using Method A.

Compound No. 72: (S)-N-[[3-[3-Fluoro[4-[3-(1α,5α,6α)-6-[N-(5-formyl-2-furyl-methyl)-N-methyl]amino]-3-azabicyclo-[3.1.0]hexane]phenyl]-2-oxo-5-oxazolidinyl ]methyl]acetamide

The title compound was made using (S)-N-[3-[3-Fluoro[4-[3-(1α,5α,6α)-6-[N-Methyl]amino]-3-azabicyclo[3.1.0]hexane]phenyl]-2-oxa-5-oxazolidinyl]acetamide and 5-formyl-2-furylmethyl chloride using Method A.

Compound No. 73: (S)-N-[[3-[3-Fluoro[4-[3-(1α,5α,6α)-6-[N-(3thienoyl)-N-methyl]amino]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]amino]-3-azabicyclo[3.1.0]hexane]phenyl]-2-oxa-5-oxazolidinyl]acetamide and 3-chlorothienoyl chloride using Method A.

Compound No. 33: (S)-N-[[3-[3-Fluoro[4-[3-(1α,5α,6α)-6-[N-(5-bromo-2-furoyl)-N-methyl]amino]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]amino]-3-azabicyclo[3.1.0]hexane]phenyl]-2-oxa-5-oxazolidinyl]acetamide and 5-bromo-2-furoyl chloride using Method A.

Method B:

General procedure was same as described earlier (Method B). Only the core amine of Formula V is (S)-N-[[3-[3-Fluoro[4-[3-(1α,5α,6α)-6-[N-methyl]amino]-3-azabicyclo[3.1.0]hexane]phenyl]-2-oxo-5-oxazolidinyl]methyl]acetamide here.

Compound No. 34: (S)-N-[[3-[3-Fluoro[4-[3-(1α,5α,6α)-6-[N-(5-nitro-2-thienyl-methyl)-N-methyl]amino]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-methyl]amino]-3-azabicyclo[3.1.0]hexane]phenyl]-2-oxa-5-oxazolidinyl]acetamide and 5-nitro-thiophene-2-carboxyaldehyde using Method B.

Compound No. 35: (S)-N-[[3-[3-Fluoro[4-[3-(1α,5α,6α)-6-[N-(5-nitro-2-furyl-methyl)-N-methyl]amino]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]amino]-3-azabicyclo[3.1.0]hexane]phenyl]-2-oxa-5-oxazolidinyl]acetamide and 5-nitro-furan-2-carboxyaldehyde using Method B.

Analogues of (S)-N-[3-[3-Fluoro[4-[3-(1α,5α,6α)-6-[N-(tert butoxy carbonyl)-N-Methyl]amino methyl]-3-azabicyclo[3.1.0]hexane]phenyl]-2-oxa-5-oxazolidinyl]acetamide (Core III).

The heteroaromatic group with the corresponding appendage can be introduced on the nitrogen atom of ring C of compounds of Formula I by one of the methods described below:

Method A:

General procedure was same as described earlier (Method A). Only the core amine of Formula V is (S)-N-[3-[3-Fluoro[4-[3-(1α,5α,6α)-6-[N-(tert butoxy carbonyl)-N-Methyl]amino methyl]-3-azabicyclo[3.1.0]hexane]phenyl]-2-oxa-5-oxazolidinyl]acetamide (core III).

Compound No. 36: (S)-N-[[3-[3-Fluoro[4-[3-(1α,5α,6α)-6-[N-(5-formyl-2-furylmethyl)-N-methyl]aminomethyl]-3-azabicyclo-[3.1.01]hexane]phenyl]-2-oxo-5-oxazolidinyl]methyl]acetamide

(a) Preparation of 3-Fluoro[4-[3-(1α,5α,6α)-6-[N-(tert butoxy carbonyl)-aminomethyl]-3-azabicyclo-[3.1.0]hexane]nitrobenzene.

(1α,5α,6α)-6-Aminomethyl-3-azabicyclo [3.1.0] hexane (7.0 g, 0.03535 mol) was taken in CH₃CN 50 mL and diisopropyl ethyl amine (4.5606 g, 0.03535 mol) was added followed by 3,4-difluoro nitrobenzene (5.6212 g, 0.03535 mol) and heated at 70° C. for 4 hrs. The reaction was monitored by the disappearance of the starting material on the (eluent CHCl₃ (19):MeOH (1). The reaction mixture was concentrated under vacuum, triturated with H₂O, filtered, washed with hexane and dried to obtain the title compound.

(b) Preparation of 3-Fluoro[4-[3-(1α,5α,6α)-6[N-(tert butoxy carbonyl)-N-Methyl]-aminomethyl]-3-azabicyclo-[3.1.0]hexane]nitrobenzene

3-Fluoro[4-[3-(1α,5α,6α)-6-[N-(test butoxy carbonyl)-N-methyl]-aminomethyl]-3-azabicyclo-[3.1.0]hexane]nitrobenzene (10 g, 0.029 mol) was taken in 60 ml THF at 0° C. Sodium hydride (1.06 g, 0.045 mol) was added portion-wise over 5 min. after complete addition the reaction mixture was stirred for 30 min. at 0° C. Methyl iodide (8.42 g, 0.059 mol) was then added over 10 min. at 0° C. followed by tat n-butyl ammonium iodide (1 g). The reaction mixture was stirred for 4 hrs. The reaction mixture was then concentrated under vacuum. H₂O (50 mL) was added followed by extraction with CH₂Cl₂ (3×50 mL). The combined organic layer was dried over Na₂SO₄, filtered and concentrated to obtain the title compound.

(c) Preparation of 3-Fluoro[4-[3-(1α,5α,6α)-6-[N-(tert butoxy carbonyl)methyl]-aminomethyl]-3-azabicyclo-[3.1.0]hexane]aniline

3-Fluoro[4-[3-(1α,5α,6α)-6-[N-(tert butoxy carbonyl)-N-Methyl]-aminomethyl]-3-azabicyclo-[3.1.0]hexane]nitro benzene (26 g, 0.074 mol) was taken in 75 mL THF and 75 mL MeOH. 10% Pd/dry (3 g) was taken in 75 ml THF and 75 mL MeOH. 10% Pd/C dry (3 g) was added and the reaction mixture was shaken in a parr hydrogenator at 40 for 3 hours. The reaction mixture was filtered through celite led. The filtrate was concentrated to obtain the title compound.

(d) Preparation of 3-Fluoro[4-[3-(1α,5α,6α)-6-[N-(tert butoxycarbonyl)-N-methyl]amino]-3-azabicyclo-[3.1.0]hexane]benzyloxy carbamate

3-Fluoro[4-[3-(1α,5α,6α)-6-[N-(tert-butoxy carbonyl)-N-methyl)amino-methyl]-3-azabicyclo [3.1.0]hexane]aniline (21 g, 0.065 mol) was taken in THF (100 ml and cooled to −15° C. Sodium bicarbonate (27.47 g, 0.32 mol) was added followed by benzyl chloroformate (14.5 g, 0.055 mol) which was added slowly over 30 min. after complete addition the stirring was combined for the maintaining the temperature between 0-5° C. The reaction was monitored by the disappearance of the reaction mixture on TLC (eluent CHCl₃:MeOH: 9:1). The reaction mixture was filtered and filtrate concentrated under vacuum. H₂O (20 ml) was added and extracted with CH₂Cl₂ (3×100 ml). The combined organic layer was dried over Na₂SO₄. This was filtered, filtrate concentrated. The semisolid was triturated with MeOH. The solid was filtered to obtain the title compound.

(e) Preparation of (S)-N-[3-[3-Fluoro[4-[3-(1α,5α,6α)-N-(tert butoxy carboxy-N-methyl]amino methyl]-3-azabicyclo[3.1.0]hexane]phenyl]-2-oxo-5-oxazolidinyl]methyl alcohol.

3-Fluoro[4-[3-(1α,5α,6α)-6-(N-(tert butoxy carbonyl)-N-methyl]amino-methyl]-3-azabicyclo[3.1.0]hexane]benzyloxy carbamate (21 g, 0.04615 mol) was taken in freshly distilled THF (200 mL). The system was thoroughly flushed with N₂. The temperature was then brought down to −78° C. in acetone dry ice. n-BuLi (59.13 mL of 15% solution in hexane, 0.13846 mol) was added over 30 min. maintaining the temperature at −78° C. The stirring was continued for 2.5 hours at −78° C. R(−) Glycidyl butyrate was added in one go and stirred at −78° C. for further 1.5 hours. The temperature was gradually increased to room temperature and stirred over night. 20% Solution of NH₄Cl (200 ml) was then added gradually added over 10 min. after 30 min. stirring, the organic layer was separated. The aqueous layer was further extracted with EtOAc (3×75 ml). The combined organic was dried over Na₂SO₄, filtered and concentrated. The product was purified by silica gel column chromatography (100-200) eluent (2% MeOH: 98% CHCl₃) to yield 14 g.

(f) Preparation of (S)-N-[3-[3-Fluoro[4-[3-(1α,5α,6α)-[N-(tert butoxy carbonyl)-N-methyl]aminomethyl]-3-azabicyclo[3.1.0]hexane]-phenyl]-2-oxa-5-oxazolidinyl]methyl methanesulfonate.

(S)-N-[3-[3-Fluoro[4-[3-(1α,5α,6α)-6-[N-(tert butoxy carbonyl)-N-methyl]aminomethyl]-3-azabicyclo[3.1.0]hexane]phenyl]-2-oxa-5-oxazolidinyl]methyl alcohol (16 g, 0.038 mol) was taken in 50 ml pyridine at 5-10° C. and methane sulphonyl chloride (12.71 g, 0.14 mol) was added over 5 min. The stirring was continued for 4 hours. The progress of the reaction was monitored by the disappearance of the starting material on TLC (eluent 10% CHCl₃: 10% MeOH). The reaction mixture was filtered, concentrated under vacuum, washed with H₂O (50 ml) and extracted with CH₂Cl₂ (3×75 mL). The combined organic layer was dried over Na₂SO₄, filtered and filtrate concentrated. This was dried thoroughly under vacuum.

(g) Preparation of (S)-N-[3-[3-Fluoro[4-[3-(1α,5α,6α)-6-[N-(tert butoxy carbonyl)-N-Methyl]amino methyl]-3-azabicyclo[3.1.0]hexane]-phenyl]-2-oxa-5-oxazolidinyl]methyl azide.

(S)-N-[3-[3-Fluoro[4-[3-(1α,5α,6α)-[N-(tert butoxy carbonyl)-N-methyl]aminomethyl]-3-azabicyclo[3.1.0]hexane]phenyl]-2-oxa-5-oxazolidinyl]methyl methane sulphonate (15 g, 0.03 mol) was taken in DMF (50 ml) and NaN₃ (9.76 g, 0.15 mol) was added and heated at 70° C. for 4 hours. The progress of the reaction was monitored by the disappearance of the starting material on TLC. The reaction mixture was filtered. The filtrate was concentrated under vacuum. This was washed with H₂O and extracted EtOAc (3×75 ml). The combined organic layer was dried over Na₂SO₄, filtered and concentrated to obtain the title compound. Yield: 11.5 g.

(h) Preparation of (S)-N-[3-[3-[3-Fluoro[4-[3-(1α,5α,6α)-6-[N-(tert butoxy carbonyl)-N-methyl]aminomethyl]-3-azabicyclo[3.1.0]hexane]-phenyl]-2-oxo-5-oxazolidinyl]methyl amine

(S)-N-[3-[3-[3-Fluoro[4-[3-(1α,5α,6α)-6-[N-(tert butoxy carbonyl)-N-methyl]aminomethyl]-3-azabicyclo[3.1.0]hexane]phenyl]-2-oxo-5-oxazolidinyl]methyl azide (11.3 g, 0.026 mol) was taken in 75 ml MeOH and 75 ml EtOAc and 10% Pd/C was added. The reaction mixture was shaken at 50 psi for 6 hrs. The progress of the reaction was monitored by the disappearance of the starting material on the TLC. The reaction mixture was filtered through a celite bed. The filtrate was concentrated. The product was triturated with diethyl ether. The solid was filtered, to obtain the title compound. Yield-7.6 g.

(i) Preparation of (S)-N-[3-[3-Fluoro[4-[3-(1α,5α,6α)-6-[N-(tert butoxy carbonyl)-N-Methyl]amino methyl]-3-azabicyclo[3.1.0]hexane]-phenyl]-2-oxa-5-oxazolidinyl]acetamide.

(S)-N-[3-[3-Fluoro[4-[3-(1α,5α,6α)-6-[N-(tert butoxy carbonyl)-N-methyl]aminomethyl]-3-azabicyclo[3.1.0]hexane]phenyl]-2-oxo-5-oxazolidinyl]methyl amine (7.6 g, 0.018 mol) was taken in pyridine (8 ml), CH₂Cl₂ (50 mL) and acetic anhydride (2.214 g, 0.0217 mol) at 0-10° C. The reaction mixture was stirred and the progress of the reaction was monitored by the disappearance of the starting material on the TLC eluent (CHCl₃:MeOH : 9:1). The reaction mixture was concentrated under vacuum. The reaction mixture was washed with H₂O (50 mL) and extracted with CH₂Cl₂ (3×50 mL). The combined organic layer was dried over Na₂SO₄, filtered and concentrated. This product was triturated with diethyl ether, filtered and dried to yield the little compound. Yield-6.6 g.

(j) Preparation of (S)-N-[3-[3-Fluoro[4-[3-(1α,5α,6α)-6-[N-methyl]aminomethyl]-3-azabicyclo[3.1.0]hexane]phenyl]-2-oxa-5-oxazolidinyl]acetamide.

(S)-N-[3-[3-Fluoro[4-[3-(1α,5α,6α)-6-[N-(tert butoxy carbonyl)-N-Methyl]amino]-3-azabicyclo[3.1.0]hexane]phenyl]-2-oxa-5-oxazolidinyl]acetamide (1 g) was taken in CH₂Cl₂ (50 mL) at 0° C. and CF₃COOH(10 ML) was added and stirred for 4 h. The reaction mixture was concentrated under vacuum. The residue was dissolved in EtOAc and neutrallised with solid NaHCO₃. The EtOAc layer was filtered and the filtrate was concentrated to obtain the title compound.

(S)-N-[[3-[3-Fluoro[4-[3-(1α,5α,6α)-6-[N-(5formyl-2-furylmethyl)-N-methyl]aminomethyl]-3-azabicyclo-[3.1.0]hexane]phenyl]-2-oxo-5-oxazolidinyl]-methyl]acetamide

The title compound was made using (S)-N-[3-[3-Fluoro[4-[3-(1α,5α,6α)-6-[N-Methyl]aminomethyl]-3-azabicyclo[3.1.0]hexane]phenyl]-2-oxa-5-oxazolidinyl]acetamide and 5-formamido-2-furylmethylene chloride using Method A.

Compound No. 37: (S)-N-[[3-[3-Fluoro[4-[3-(1α,5α,6α)-6-[N-(5-carboxyethyl-2-furylmethyl)-N-methyl]aminomethyl]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]aminomethyl]-3-azabicyclo[3.1.0]hexane]phenyl]-2-oxa-5-oxazolidinyl]acetamide and ethyl-5-(chloromethyl)-2-furan carboxylate using Method A.

Compound No. 38: (S)-N-[[3-[3-Fluoro[4-[3-(1α,5α,6α)-6-[N-(2-thiophene-acetyl)-N-methyl]aminomethyl]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]amino]-3-azabicyclo[3.1.0]hexane]phenyl]-2-oxa-5-oxazolidinyl]acetamide and 2-thiopheneacetyl chloride using Method A.

Method-B:

General procedure was same as described earlier in section 7.1.1.2. (Method B) described earlier. Only the core amine of Formula V is (S)-N-[3-[3-Fluoro[4-[3-(1α,5α,6α)-6-[N-Methyl]aminomethyl]-3-azabicyclo[3.1.0]hexane]phenyl]-2-oxa-5-oxazolidinyl]acetamide (Core III)

Compound No. 39: (S)-N-[[3-[3-Fluoro[4-[3-(1α,5α,6α)-6-[N-(5-nitro-2-thienyl-methyl)-N-methyl]aminomethyl]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]aminomethyl]-3-azabicyclo[3.1.0]hexane]phenyl]-2-oxa-5-oxazolidinyl]acetamide and 5-nitro thiophene-2-carboxyaldehyde using Method B.

Compound No. 40: (S)-N-[[3-[3-Fluoro[4-[3-(1α,5α,6α)-6-[N-(5-nitro-2-furyl-methyl)-N-methyl]aminomethyl]-3-azabicyclo-[3.1.0]hexane]phenyl]-2-oxo-5-oxazolidinyl]methyl]acetamide

The title compound was made using (S)-N-[3-[3-Fluoro[4-[3-(1α,5α,6α)-6-[N-Methyl]aminomethyl]-3-azabicyclo[3.1.0]hexane]phenyl]-2-oxa-5-oxazolidinyl]acetamide and 5-nitro-furan-2-carboxyaldehyde using Method B.

Analogues of (S)-N-{3-[4-[4-N-methyl amino peperidin-1-yl]-3-fluorophenyl}-2-oxo-oxazolidin-5-yl]methyl acetamide (Core IV).

The heteroaromatic group with the corresponding appendage can be introduced on the nitrogen atom of ring C of compounds of Formula I by one of the methods described below:

Method-A:

General procedure was same as described earlier (Method A). Only the amine of Formula V is (S)-N-{3-[4-[4-N-methyl amino piperidin-1-yl]-3-fluorophenyl}-2-oxo-oxazolidin-5-yl]methyl acetamide (core IV).

Compound No. 74:Preparation of (S)-N-[[3-[4-[4-(N-methyl-N-2furyl(5-formyl)-methylaminopiperidine-1-yl]-3-fluorophenyl]-2-oxo-oxazolidin-5-yl]methyl]-acetamide.

Preparation of (S)-N-{3-[4-[4-N-methyl amino piperidin-1-yl]-3-fluorophenyl}-2-oxo-oxazolidin-5-yl]methyl acetamide (Core IV)

(a) 1-[4(N-t-Butyloxycarbonylamino)piperidin-1-yl]-3-fluoro]-nitrobenzene

To a solution of 1,2-difluoro-4-nitrobenzene (40 g; 200 mmol) in acetonitrile (400 ml) was treated with ethyldiisopropyl amine (28.4 g; 219.72 mmol) and 4-(t-butyloxycarbonyl) amino piperidine (31.8 g; 199 mmol). The whole reaction mixture was then heated at 60° C. for 6.0 hr. The solution was cooled to ambient temperature and conc. in vacuo. The residue was dissolved in ethyl acetate and washed with water. Ethyl acetate layer was dried over anhydrous sodium sulphate. Solvent was removed to afford a yellow solid (60 g).

δppm (CDCl₃) : 7.98-7.80 (m, 2H), 6.91 (t, J=9 Hz, 1H) 4.53 (bs, 1H), 3.65 (d, J=12 Hz, 3H) 2.98 (t, J=13 Hz, 2H), 2.07 (m, 2H), 1.69-1.53 (m, 3H), 1.52 (s, 9H).

(b) 1-[4-(N-t-Butyloxy carbonyl N methyl)aminopiperidin-1-yl]-3-fluoro]nitrobenzene (B)

To a solution of intermediate A (89 mmol) in dry tetrahydrofuran (400 ml) was added sodium hydride (60%, 106 mmol) in cold condition (0° C.) followed by tetrabutyl ammonium iodide (10 mmol). The reaction mixture was stirred at cold to r.t. for 2.0 hr. Methyl iodide (267 mmol) was then added at 0° C. Reaction mixture was stirred at r.t. for 12 hr. A faster moving spot was appeared. Excess sodium hydride was decomposed with water. Tetrahydrofuran was removed. The residue was dissolved in ethyl acetate, washed with water, brine and then with water. Organic layer was dried over anhydrous sodium sulphate and solvent was removed. A yellow solid (32 g) was obtained.

δppm (CDCl₃): 6.81 (t, J=12 Hz, 1H) 6.44-6.37 (m, 2H), 4.70 (bs, 1H) 2.91 (d, J=12H, 2H), 2.78 (s, 3H), 2.72-2.65 (m, 2H), 1.47 (s, 9H).

(c) 1-[4-[(N-t-butyloxycarbonyl-N-methyl)amino-piperidin-1-yl]-fluoro]aniline (C)

A mixture of nitro compound B, (32.0 g ), 3.2 g of 10% palladium on carbon in 75 ml of methanol was shaken in a Paar shaker flask under 40 Psi hydrogen for 6.0 hr. TLC showed a slower moving spot. The reaction mixture was filtered through celite. Solvent was removed. A dark solid was obtained (28.6 g), it was subjected to next step without further characterisation.

(d) 1-{N-Carbobenzyloxy-[4-[(N-t-butyloxy carbonyl-N-methyl)-peperidin-1-yl]}-3-fluoro]aniline (D)

To the solution of aniline derivative C (19.0 g, 58.823 mmol) in dry tetrahydrofuran (150 ml) was added. Sodium hydrogen carbonate (19.76 g, 235.29 mmol). It was cooled to 0° C. and benzyl chloroformate (12.9 ml, 50% toluene sol.) was added. The whole reaction mixture was stirred at 0° C.-r.t. for 6.0 hr. TLC showed faster moving spot compare to aniline derivative. Reaction mixture was filtered through celite. Solvent removed. Residue was digested with hexane and solvent was removed to give 23.4 g of CBz derivative.

δppm (CDCl₃) : 7.39-7.28 (m, 6H), 6.99-6.86 (m, 2H), 6.75 (bs, 1H), 5.20 (s, 2H), 4.20 (bs, 1H), 3.43 (d, J=12 Hz, 2H), 2.79 (s, 3H), 2.71 (m, 2H), 1.97-1.86 (m, 2H), 1.49 (s, 9H)

(e) (S)-N-{3-[4-[4-(N-methyl-N-t-butyloxy carbonyl)amino-piperidin-1-yl]-3-fluorophenyl}-2-oxo-oxazolidin-5-yl]methanol (E)

To a solution (200 ml) of CBz derivative in (D; 24.0 g, 52.516 mmol) dry tetrahydrofuran was added. BuLi (67 ml, 157 mmol) at −78° C. under N₂. The reaction mixture was stirred at −78° C. for 2.0 hr. Glycidyl butyrate (9.07 g, 62.98 mmol )was then added to the reaction mixture at −78° C. It was stirred at −78° C. for 1 hr. then allowed to reach r.t. TLC of the reaction mixture showed a slower moving spot. Ammonium chloride (30 ml) was added to the reaction mixture. It was stirred for 5 min. Ammonium chloride layer was separated and extracted with ethyl acetate. Tetrahydrofuran and ethyl acetate layer were combined, dried over anhydrous sodium sulphate. Solvent was removed. The residue was purified by column chromatography using CHCl₃:MeOH (1.5%-2.5% )as eluent to give 10 g of desired alcohol.

δppm (CDCl₃): 7.46 (d, J=8.0 Hz, 1H), 7.10 (d, J=9 Hz, 1H), 6.94 (t, J=9 Hz, 1H) 4.55 (bs, 1H), 4.07-3.87 (m, 5H), 3.74 (bs, 1H), 3.46 (bs, 1H), 3.42 (bs, 1H), 2.78-2.89 (m, 5H), 1.96-1.85 (m, 2H), 1.72 (s, 1H), 1.47 (s, 9H).

(f) (S)-N-{3-[4-[4-(N-Methyl-N-t-butyloxy carbonyl)aminopiperidin-1-yl)-3-fluorophenyl}-2-oxo-oxazolidine-5-yl}methyl methane sulfonate (F)

To a solution of hydroxymethyl compounds (E, 24 g, 56.73 mmol) in dichloromethane (400 ml) was added triethylamine (11.46 g, 113.46 mmol) followed by methane sulphonyl chloride at 0° C. The reaction mixture was stirred at 0° C.—r.t. for 3.0 hr. TLC of the reaction mixture showed a faster moving spot. The reaction mixture was poured in to water and extracted with dichloromethane, washed with saturated sodium bicarbonate solution and then with water. Organic layer was dried over anhydrous sodium sulphate and solvent was removed to give 28.4 g of compound (F).

δppm CDCl₃): 7.45 (d, J=12 Hz, 1H), 7.10-7.01 (m, 2H), 4.92 (bs, 1H), 4.53-4.40 (m, 2H), 4.12(t, J=9 Hz, 1H), 7.10-7.01 (m, 2H), 4.12 (t, J=9 Hz, 1H), 3.94-3.89 (m, 1H), 3.48 (d, J=12 Hz, 2H), 3.15 (m, 1H), 3.11 (s, 3H) 2.79 (s, 3H), 1.97-193 (m, 2H), 1.77-1.69 (m, 4H), 1.48 (s, 9H).

(g) (S)-N-{3-[4-[4-(N-Methyl-N-t-butyloxy carbonyl)aminopiperidin-1-yl)-3-fluorophenyl]-3-fluorophenyl]-2-oxo-oxazolidin-5-yl}methyl azide (G)

To the solution of mesyl derivative (F, 28.4 g, 56.68 mmol) in dimethyl formamide (350 ml) was added sodium azide (11.059, 70.05 mmol). The whole reaction mixture was heated at 80° C. for 9.0 hr. TLC showed a faster moving spot. Reaction mixture was filtered. Dimethyl formamide was removed in reduced pressure. The residue was digested in hexane to afford desired azide in 26.0 g.

δppm CDCl₃): 7.44 (d, 12 Hz, 1H), 7.11 (bs, 1H), 6.97 (t, J=9 Hz, 1H) 4.78 (bs, 1H), 4.09-3.49 (m, 7H), 2.90 (s, 3H), 2.75 (bs, 2H) 1.49 (s, 9H).

(h) (S)-N-{3-[4-[4-(N-Methyl-N-t-butyloxy carbonyl)aminopiperidin-1-yl)-3-fluorophenyl]-2-oxo-oxazolidin-5-yl methyl amine (H)

To the solution of azido compound (G, 25.5 g, 56.92 mmol) in methanol (50 ml) was added, 10% Pd/C (2.5 g). The whole reaction mixture was hydrogenated for 10 hr. at 40 Psi. TLC showed a slower moving spot. It was filtered through celite bed and solvent was removed to give desired product of 24.5 g.

δppm CDCl₃): 7.45 (d, J=12 Hz, 1H), 7.11 (d, J=9 Hz, 1H), 6.94 (t, J=9 Hz, 1H) 4.66 (bs, 1H), 4.00 (t, J=9 Hz, 1H), 3.81 (t, J=9 Hz, 1H), 3.45 (d, J=9 Hz, 2H) 3.10-2.90 (m, 1H), 2.78 (3 3H), 2.73 (bs, 1H), 1.48 (s, 9H).

(i) (S)-N-{3-[4-[4-(N-Methyl, N-1-butyloxy carbonyl) amino piperidin-1-yl)-3-fluorophenyl]-2-oxo-oxazolidin-5-yl}methyl acetamide (I)

To a solution of methyl amino derivative (7.0 g, 16.58 mmol) in dichloro methane (120 ml) was added triethyl amine (2.18 g; 21.58 mmol) reaction mixture was cooled to 0° C. and acetic anhydride was added slowly. It was stirred at 0°—r.t. for 5.0 hr. TLC showed a faster moving spot. Reaction mixture was poured into water and extracted with dichloromethane. Organic layer was washed with sodium bicarbonate, brine and water. Organic layer was dried over anhydrous sodium sulphate and solvent was removed to give 7.1 g of crude desired product which on purification gave 4.1 g of pure product.

δppm CDCl₃): 7.43 (d, J=12 Hz, 1H), 7.07 (d, J=9 Hz, 1H), 6.95 (t, J=9 Hz, 1H) 6.28 (bs, 1H), 4.00 (t, J=9 Hz, 1H), 3.78-3.62 (m, 3H), 3.47 (d, J=9 Hz, 2H) 2.80 (s, 3H), 2.75-2.71 (m 2H), 2.03 (s, 3H), 1.49 (s, 9H). (j) (S)-N-[3-[4-[4-N-methyl)amino piperidin-1-yl]-3-fluorophenyl]-2-oxo-oxazolidin-5-yl]methyl acetamide (J)

To a solution of Boc protected compound (I, 2.0 g, 4.31 mmol) in dichloromethane (35 ml) was added trifluoroacetic acid (5 ml) at 0° C. The whole reaction mixture was stirred at 0° r.t. for 3 hr. TLC of the reaction mixture showed a slower moving spot. Solvent was removed and the residue was dissolved in acetone, anhydrous pot. Carbonate was added to neutralize trifluoro acetic acid. It was stiffed at r.t. for 2.0 min. then filtered through a Buckner funnel. Solvent was removed and the title compound was obtained. Yield: 1.5 g

Compound No. 41: (S)-N-[[3-[4-[4-(N-methyl-N-2furyl(5formyl)methyl-aminopiperidine-1-yl]-3-fluorophenyl]-2oxo-oxazolidin-5-yl]methyl]acetamide

The title compound was made using (S)-N-[[3-[4-[4-(N-methyl-)amino piperidine-1-yl]-3-fluorophenyl]-2oxo-oxazolidin-5-yl]methyl]acetamide and 5-chloromethyl -2-furfural following Method A.

Compound No. 42: (S)-N-[[3-[4-[4-(N-methyl-N-(3,5-difluorobenzoyl)amino-piperidine-1-yl]-3-fluorophenyl]-2oxo-oxazolidin-5-yl]methyl]acetamide.

The title compound was made using (S)-N-[[3-[4-[4-(N-methyl-)amino piperidine-1-yl)-3-fluorophenyl]-2oxo-oxazolidin-5-yl]methyl]acetamide and 3,5, difluoro benzoyl chloride following Method A.

Compound No. 43: (S)-N-[[3-[4-[4-(N-methyl-N-(5-bromo-2-furoyl)amino-piperidine-1-yl]-3-fluorophenyl]-2oxo-oxazolidin-5-yl]methyl]acetamide

The title compound was made using (S)-N-[[3-[4-[4-(N-methyl-)amino piperidine-1-yl]-3-fluorophenyl]-2oxo-oxazolidin-5-yl]methyl]acetamide and 5-bromo-2-furoyl chloride following Method A.

Compound No. 44: (S)-N-[[3-[4-[4-(N-methyl-N-(5-nitro-2-furoyl)amino-piperidine-1-yl]-3-fluorophenyl]-2oxo-oxazolidin-5yl]methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and 5-nitro-2-furoyl chloride following Method A.

Compound No. 45: (S)-N-[[3-[4-[4-(N-methyl-N-3-furoyl)aminopiperidine-1-yl]-3-fluorophenyl]-2oxo-oxazolidin-5-yl]methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and 3-furoyl chloride using Method A.

Compound No. 46: (S)-N-{3-[4-[4-(N-methyl, N-2-furoyl )aminopiperidine-1-yl]-3-fluorophenyl]-2oxo-oxazolidin-5-yl methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and 2-furoyl chloride following Method A.

Compound No. 47:(S)-N-{3-[4-[4-(N-methyl, 2-thiopheneacetyl)aminopiperidine-1-yl]-3-fluorophenyl]-2oxo-oxazolidin-5-yl methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and 2-thiophene acetylchloride chloride following Method A.

Method-B:

General procedure was same as described earlier in section (Method B), only the amine of Formula V is (S)-N-[[3-[4-[4-(N-methyl-)amino piperidine-1-yl]-3-fluorophenyl]-2oxo-oxazolidin-5-yl]methyl]acetamide (core IV).

Compound No. 48:(S)-N-[[(3-[4-[4-(N-methyl-N-2furylmethyl) aminopiperidine-1-yl]-3-fluorophenyl]-2oxo-oxazolidin-5-yl]methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and furan-2-carboxaldehyde following Method B.

Compound No. 49: (S)-N-[[3-[4-[4-(N-methyl-N-3-furyl)aminopiperidine-1-yl]-3-fluorophenyl]-2oxo-oxazolidin-5-yl]methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and furan-3-carboxaldehyde following Method B.

Compound No. 50: (S)-N-[[3-[4-[4-(N-methyl-N-2-furyl(5-nitro)methyl)-aminopiperidine-1-yl]-3-fluorophenyl]-2oxo-oxazolidin-5-yl]methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and 5-nitro furan-2-carboxaldehyde using Method B.

δppm (CDCl₃): 7.40(d, 1H), 7.29 (m, 1H), 7.29 (m, 1H), 7.05 (dd, 1H), 6.92 (t, 1H), 6.48 (d, 1H), 6.26 (bs, 1H), 4.76 (bs, 1H), 4.01 (t, 1H), 3.77-3.60 (m, 5H), 3.47 (d, 2H), 2.66 (t, 3H), 6.26 (bs, 1H), 4.76 (bs, 1H), 4.01 (t, 1H), 3.77-3.60 (m, 5H), 3.47 (d, 2H), 2.66 (t, 3H), 6.26 (bs, 1H), 4.76 (bs, 1H), 4.01 (t, 1H), 3.77-3.60 (m, 5H), 3.47 (d, 2H), 2.66 (5, 3H), 2.37 (s, 3H), 2.01 (s, 3H, 1.93-1.25 (m, 4H).

Compound No. 51: (S)-N-[[3-[4-[4-(N-methyl-N-2-thienyl(5-nitro)methyl)-aminopiperidine-1-yl]-3-fluorophenyl]-2oxo-oxazolidin-5-yl]methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and 5-nitro thiophen-2-carboxaldehyde following Method B.

δppm (CDCl₃): 7.79 (d, 1H), 7.41 (dd, 1H), 7.05 (d, 1H) 6.93 (t, 1H), 6.85 (d, 1H), 6.11 (bs, 1H), 4.01 (t, 1H) 3.82-3.45 (m, 7H), 2.66 (m, 3H), 2.37 (s, 3H), 2.02 (s, 3H) 1.82-1.25 (m, 4H)

Compound No. 52: (S)-N-[[3-[4-[4-(N-methyl-N-2-thienylmethyl)aminopiperidine-1-yl]-3-fluorophenyl]-2oxo-oxazolidin-5-yl]methyl]acetamide

The title compound was made using (S)-N-[[3-[4-[4-(N-methyl-)amino piperidine-1-yl]-3-fluorophenyl]-2oxo-oxazolidin-5-yl]methyl]acetamide and thiophen-2-carboxaldehyde following Method B.

Compound No. 53: (S)-N-[[3-[4-[4-(N-methyl-N-(5-methyl-2-thienylmethyl)aminopiperidine-1-yl]-3-fluorophenyl]-2oxo-oxazolidin-5-yl]methyl]-acetamide

The title compound was made using (S)-N-[[3-[4-[4-(N-methyl-)amino piperidine-1-yl]-3-fluorophenyl]-2oxo-oxazolidin-5-yl]methyl]acetamide and 5-methyl-thiophen-2-carboxaldehyde following Method B.

Compound No. 54: (S)-N-{3-[4-[4-(N-methyl,2-(5-bromo)thienylmethyl)amino-piperidine-1-yl]-3-fluorophenyl]-2oxo-oxazolidin-5-yl methyl]acetamide.

The title compound was made using (S)-N-[[3-[4-[4-(N-methyl-)amino piperidine-1-yl]-3-fluorophenyl]-2oxo-oxazolidin-5-yl]methyl]acetamide and 5-bromo,-thiophen-2-carboxaldehyde Method B.

Analogues of of (S)-N-[[3-[3[Fluoro-4-(N-1-homopiperazinyl)phenyl]-2-oxo-5-oxazolidinyl]methyl]acetamide (Core V)

The heteroaromatic group with the corresponding appendage can be introduced on the nitrogen atom of ring C of compounds of Formula I by one of the methods described below:

Method A:

General procedure was same as described earlier (Method A). Only the core amine of Formula V is (S)-N-{3-[4-[4-N-methylaminopeperidin-1-yl]-3-fluorophenyl}-2-oxo-oxazolidin-5-yl]methyl acetamide (core V).

Compound No. 55: (S)-N[[3-[3-Fluoro-4-[N-1[4-{2-furyl(5-formyl)methyl}]-homopiperazinyl]phenyl]2-oxo-5-oxazolidinyl]methyl]acetamide

Preparation of (S)-N-[[3-[3[Fluoro-4-(N-1-homopiperazinyl)phenyl]-2-oxo-5-oxazolidinyl]methyl]acetamide

(a) Preparation of 1-(2-Fluoro-4-nitrophenyl)homopiperazine.

To homopiperazine (5 g, 0.05 mol) in acetonitrile (30 mL), 3,4-difluoronitrobenzene (3.17 g, 0.02 mol) was added and the reaction mixture was heated to reflux for 4 hrs. Then the solvent was evaporated and the residue taken in EtOAc and washed with water and brine solution. The EtOAc layer was dried over anhyd Na₂SO₄ and evaporated in vacuo. The residue was digested with ether-hexane (1:20), decanted and dried in vacuo to get 3.7 g of final product.

δppm (CDCl₃): 7.9 (m, 2H, Ar—H), 6.75 (t, 1H, A—H) 3.64 (m, 4H, CH₂), 3.08 (m, 2H, CH₂), 2.91 (m, 2H, CH₂), 1.96 (m, 2H, CH₂).

(b) Preparation of 1-(2-Fluoro-4-nitrophenyl)-4-tert-butoxycarbonyl-homopiperazine.

To 1-(2-Fluoro-4-nitrophenyl)homopiperazine (3.5 g, 14.6 mmol) in dichloromethane (100 mL) cooled to 5° C., triethylamine (0.2 mL, 1.46 mmole) and tert-butoxydicarbonate (4.15 g, 19.03 mmol) was added and the reaction mixture was stirred for 18 hrs. The solvent was evaporated and to the residue hexane was added. The product precipitating out was filtered, washed with hexane and dried in air to yield 4.0 g of the final product.

δppm CDCl₃): 7.93 (m, 2H, Ar—H), 6.78 (t, 1H, Ar—H), 3.63 (m, 6H, CH₂), 3.43 (m, 2H, CH₂), 1.97 (m, 2H, CH₂), 1.50 (s, 9H, t-Bu).

(c) 3-Fluoro-4-(N-tert-butoxycarbonylhomopiperazinyl)aniline.

To 1-(2-Fluoro-4-nitrophenyl)-4-tert-butoxycarbonylhomopiperazine (3.2 g, 9.4 mmole) in methanol (30 mL), 10% palladium/carbon was added and shaken in a Parr hydrogenation apparatus under 40 psi of hydrogen gas for 3 hrs. Then the reaction mixture was filtered over celite and the filtrate evaporated in vacuum to yield 2.64 g of the final product.

δppm (CDCl₃): 6.81 (t, 1H, Ar—H), 6.38 (m, 2H, Ar—H) 3.53 (m, 4H, CH₂) 3.21 (m, 4H, CH₂), 2.86 (br s, NH₂), 1.95 (m, 2H, CH₂), 1.45 (s, 9H, t-Bu).

(d) N-Benzyloxycarbonyl-3-fluoro-4-(N-tert-butoxylcarbonylhomo-piperazinyl)aniline.

To 3-Fluoro-4-(N-tert-butoxycarbonylhomopiperazinyl)aniline (2.6 g, 8.4 mmol) in THF (25 ml) cooled to 5° C., sodium bicarbonate (0.85 g 10.1 mmol), was added and then benzylchloroformate (1.72 g, 10 mmol) was added dropwise. The reaction mixture was stirred for 18 hrs. at R.T. and then filtered. The filtrate was evaporated in vacuo. The residue was dissolved in dichloromethane and washed with saturated sodium bicarbonate solution and brine water. The organic layer was dried over anhyd Na₂SO₄ and evaporated in vacuo to give 5.04 g of final product.

δppm (CDCl₃): 7.35 (s, 6H, Ar—H), 6.84 (m, 2H, Ar—H), 6.54 (s, 1H, NH), 5.17 (s, 2H, CH₂), 3.2-3.61 (m, 8H, CH₂), 1.93 (m, 2H, CH₂), 1.45 (s, 9H, t-Bu).

(e) (R)-[N-3-[3-Fluoro-4-[N-1-(4-tert-butoxycarbonyl)homopiperazinyl]phenyl]-2-oxo-5-oxazolidinyl]methanol

To N-benzyloxycarbonyl-3-fluoro-4-(N-tert-butoxycarbonylhomopiperazinyl)aniline (2.5 g, 5.6 mmol) dissolved in dry THF(25 mL), cooled to −78° C., butyl lithium(4.8 mL, 15% sol. in hexane, 11.3 mmol) was added under +ve pressure of nitrogen. The reaction mixture was stirred at −78° C. for 1.5 hrs. Then R-glycidyl butyrate (0.89 g, 6.2 mmol) was added and the reaction mixture was stirred at −78° C. for 1 hr and then at R.T. for 18 hrs. To it 25 mL of satd ammonium chloride solution was added and the reaction mixture extracted with EtOAc. The combined organic layers were washed with water and brine water, dried over anhydrous Na₂SO₄ and evaporated in vacuo. The crude product (˜3 g) was purified by column chromatography (3% MeOH/CHCl₃) to yield 0.41 g of final product.

δppm CDCl₃) : 7.38 (d, 1H, ArH), 7.04 (d, 1H, Ar—H), 6.87 (t, 1H, Ar—H), 4.72 (m, 1H, CH), 4.1-3.2 (m, 11H, CH₂), 2.18 (br s, 1H), 1.94 (m, 2H, CH₂), 1.45 (s, 9H, t-Bu).

(f) (R)-[N-3[3-Fluoro-4-[N-1-(4-tert-butoxycarbonyl)homopiperazinyl]phenyl]-2-oxo-5-oxazolidinyl]methyl methanesulfonate.

To the (R)-[N-3[3-Fluoro-4-[N-1-(4-tert-butoxycarbonyl)homopiperazinyl]-phenyl]-2-oxo-5-oxazolidinyl]methanol (1.55 g, 3.8 mmol) in dichloromethane (10 mL) cooled to 5° C., triethylamine (0.76 g, 7.6 mmol) and methanesulfonylchloride (0.6 g, 5.3 mmoles) were added and the reaction mixture was stirred for 1 hr. Then the reaction mixture was diluted with dichloromethane and washed with saturated sodium bicarbonate solution and brine. The organic layer was dried over anhydrous sodium sulfate and evaporated in vacuo to yield 1.39 of product.

δppm (CDCl₃): 7.32 (d, 1H, ArH), 7.02 (d, 1H, Ar—H), 6.87 (t, 1H, Ar—H), 4.89 (m, 1H, CH), 4.47 (m, 2H, CH₂), 4.09 (t, 1H, CH), 3.89 (m, 1H, CH), 3.65-3.2 (m, 8H, CH₂), 3.1 (s, 3H, CH₃), 1.94 (m, 2H, CH₂), 1.45 (s, 9H, t-Bu).

(g) (R)-[N-3[3-Fluoro-4-[N-1-(4-tert-butoxycarbonyl)homopiperazinyl]phenyl]-2-oxo-5-oxazolidinyl]methylazide.

To (R)-[N-3[3-Fluoro-4-[N-1-(4-tert-butoxycarbonyl)homopiperazinyl]-phenyl]-2-oxo-5-oxazolidinyl]methyl methanesulfonate compound (1.21 g, 2.5 mmoles) in DMF(10 mL), sodium azide (0.81 g, 12 mmoles) was added and the reaction mixture heated to 80° C. for 5 hrs. The solid was filtered off and the filterate evaporated in vacuo. The residue was dissolved in chloroform and washed with water and brine solution. The organic layer was dried over anhyd. Na₂SO₄ and evaporated in vacuo to yield 1.2 g of the product.

δppm (CDCl₃): 7.32 (d, 1H, Ar—H), 7.04 (d, 1H, Ar—H), 6.87 (t, 1H, Ar—H), 4.75 (m, 1H, CH), 4.02 (t,1H, CH), 3.8-3.2(m, 1H, CH₂), 1.92 (M, 2H, CH₂), 1.45 (s, 9H, t-Bu).

(h) (R)-N-3-[3-Fluoro-4-[N-1-(4-tert-butoxycarbonyl)homopiperazinyl]phenyl]-2-oxo-5-oxazolidinyl]methylamine.

To (R)-[N-3[3-Fluoro-4-[N-1-(4-tert-butoxycarbonyl)homopiperazinyl]phenyl]-2-oxo-5-oxazolidinyl]methylazide (1.1 g, 2.5 mmol) in methanol (10 mL), 10% palladium/carbon (0.22 g) was added and the reaction mixture shaken in a Parr hydrogenation apparatus under 40 psi hydrogen pressure for 5 hrs. The reaction was filtered over celite and the filterate evaporated in vacuo to yield 0.9 g of product. The product was used as such in next step without further purification and characterization.

(i) (S)-N-[[3-[3-Fluoro-4-[N-1-(4-tert-butoxycarbonyl)homopiperazinyl]phenyl]2-oxo-5-oxazolidinyl]methyl]acetamide.

To (R)-[N-3-[3-Fluoro-4-[N-1-(4-tert-butoxycarbonyl)homopiperazinyl]phenyl]-2-oxo-5-oxazolidinyl]methylamine (0.77 g, 1.9 mmol) in dichloromethane (10 mL), triethylamine (0.21 g, 2.17 mmol) and acetic anhydride (0.21 g, 2 mmol) were added and the reaction mixture was stirred at R.T. for 30 minutes. Then the reaction mixture was diluted with dichloromethane and washed with saturated sodium bicarbonate solution and brine water. The organic layer was dried over anhydrous sodium sulfate and evaporated in vacuo. The residue was purified by column chromatography (2% MeOH(CHCl₃) to yield 0.48 g of final product.

δppm CDCl₃): 7.35(d, 1H, Ar—H), 7.02(d,1H, Ar—H), 6.86(t, 1H, Ar—H), 5.96(t, 1H, NH), 4.73(m, 1H, CH), 3.99(t, 1H, CH), 3.25-3.8(m, 1H, CH₂), 2.01(s, 3H. CH₃), 1.95(m, 2H, CH₂), 1.44(s, 9H, t-Bu).

(j) (S)-N-[[3-[3[Fluoro-4(N-1-homopiperazinyl)phenyl]-2-oxo-5-oxazolidinyl]methyl acetamide

To (S)-N-[[3-[3-Fluoro-4-[N-1-(4-tert-butoxycarbonyl)homopiperazinyl]phenyl]2-oxo-5-oxazolidinyl]methyl]acetamide (0.5 g, 1.11 mmol) in dichloromethane (8 mL), trifluoroacetic acid (2 mL) was added and stirred for 2 hrs. Then the reaction mixture was evaporated and dried in vacuo. To the residue in acetone (10 mL), potassium carbonate (0.78 g, 5.55 mmol) was added and stirred for 15 mts. Then the reaction mixture was filtered and the filterate evaporated in vacuo to yield the product in quantitative yield. This product was used as such in next step without further characterization.

Compound No. 55: (S)-N[[3-[3-Fluoro-4-[N-1[4-{2-furyl(5-formyl)methyl}]homopiperazinyl]phenyl]2-oxo-5-oxazolidinyl]methyl]acetamide

The title compound was made with (S)-N-[[3-[3[Fluoro-4-(N-1-homopiperazinyl)phenyl]-2-oxo-5-oxazolidinyl]methyl acetamide and 2-form yl-5-chloro-methylfuran using Method A.

δppm (CDCl₃): 9.61(s, 1H, CHO), 7.35(d, 1H, Ar—H), 7.2(d, 1H, Ar—H), 7.02(d, 1H, Ar—H), 6.83(t, 1H, Ar—H), 6.48(s, 1H, Ar—H), 5.96(t, 1H, NH), 4.72(m, 1H, CH), 4.71(t, 1H, Ar—H), 4.14 (s, 1H, CH₂), 3.2-3.8(m., 7H, CH₂), 2.8-3(m, 4H, CH₂), 2.09(m, 5H, CH₂, CH₃)

Compound No. 56: (S)-N[[3-[3-Fluoro-4-[N-1[4-(2-thienylacetyl)]homopiperazinyl]phenyl]2-oxo-5-oxazolidinyl]methyl]acetamide

The title compound was made with (S)-N-[[3-[3[Fluoro-4-(N-1-homopiperazinyl)phenyl]-2-oxo-5-oxazolidinyl]methyl]acetamide and 2-thiophenacetyl-chloride using Method A.

δppm CDCl₃): 7.34(m, 1H, Ar—H), 7.18(t, 1H, Ar—H), 7.2-6.78(m, 4H, Ar—H), 6.22(t, 1H, NH), 4.74(m, 1H, CH), 4.2-3.52(m, 10H, CH₂), 3.52-3.15(m, 4H, CH₂), 2.01(m, 5H, CH₂, CH₃)

Compound No. 57: (S)-N[[3-[3-Fluoro-4-[N-1[4-{2-thienyl(5-nitro)methyl}]homopiperazinyl]phenyl]2-oxo-5-oxazolidinyl]methyl]acetamide

The title compound was made with (S)-N-[[3-[3[Fluoro-4-(N-1-homopiperazinyl)phenyl]-2-oxo-5-oxazolidinyl]methyl acetamide and 5-nitro-2-thiophencarboxaldehyde using Method B.

δppm (CDCl₃): 7.78(s, 1H, Ar—H), 7.35(d, 1H, Ar—H), 7.04(m, 1H, Ar—H), 6.87(m, 2H, Ar—H), 5.99(t, 1H, Ar—H), 4.75(m, 1H, CH), 4.0(t, 1H, CH), 3.85(s, 2H, CH₂), 3.52-3.8(m, 3H, CH₂), 3.42(m, 4H, CH₂), 2.9-2.75(m, 4H, CH₂), 2.01(m, 5H, CH₂, CH₃)

Compound No. 58: (S)-N[[3-[3-Fluoro-4-[N-1[4-(3-furylmethyl)]homopiperazinyl]phenyl]2-oxo-5-oxazolidinyl]methyl]acetamide

The title compound was made with (S)-N-[[3-[3[Fluoro-4-(N-1-homopiperazinyl)phenyl]-2-oxo-5-oxazolidinyl]methyl]acetamide and 3-furaldehyde using Method B.

δppm (MeOD): 7.71 (s, 1H, Ar—H), 7.59(s, 1H, Ar—H), 7.45(d, 1H, Ar—H), 7.12(d, 1H, Ar—H), 7.01(t, 1H, Ar—H), 6.6(s, 1H, Ar—H), 4.53(m, 8H, CH₂), 4.1(m, 2H, CH₂), 3.77(t, 1H, CH), 3.75-3.45(m, 5H, CH₂), 2.19(m, 2H, CH₂), 1.96(s, 3H, CH₃)

SCHEME-II

Compound No. 59: Preparation of (S)-N-[[3-[3-fluoro-4-[N-1{2-furyl-[4-(5-difluoromethyl) methyl}]piperazinyl]-2-oxo-5-oxazolidinyl]-methyl]acetamide.

To a solution of (S)-N-[[3-Fluoro-4-[N-1[4-{2-furyl(5-formyl)methyl}]piperazinyl]phenyl]-2-oxo-5-oxazolidinyl]methyl]acetamide (80 mg, 0.18 mmol) in dichloromethane (4.0 ml) was added diethylamino sulfurtrifluoride (58 mg, 0.35 mmol). The whole reaction mixture was stirred at r.t. for 12 hr. TLC of the reaction mixture showed a faster moving spot. It was poured into a container and extracted with dichloromethane. Dichloromethane layer was washed with water, dried over anhydrous sodium sulphate. Solvent was removed. A gummy compound (60 m) was obtained.

δppm CDCl₃): 7.44 (d, 1H), 7.05 (d, 1H), 6.92 (t, 1H) 6.62 (m, 2H), 6.36 (m, 1H), 6.12 (bs, 1H), 4.60 (bs, 1H), 3.24-2.95(m, 6H), 2.74), 2.74 (bs, 4H) 4.01 (m, 1H) 3.68 (m, 3H), 2.00 (s, 3H).

Compound No. 74: Preparation of (S)-N-[[3-[3-fluoro-4-[N-1{2-furyl-[4-(5-fluoromethyl) methyl}]piperazinyl]-2-oxo-5-oxazolidinyl]-methyl]acetamide.

The title compound was made from (S)-N-[[3-[3-Fluoro-4-[N-1{2-furyl-[4-(5-hydroxymethyl)methyl}]piperazinyl]-2-oxo-5-oxazolidinyl]methyl]acetamide by using the procedure mentioned for Compound No. 59.

Compound No. 60: (S)-N-[[3-[3-Fluoro-4-[N-1-[4-(2-furyl-(5-aldoxime)methyl}]piperazinyl]phenyl]-2-oxo-5-oxazolidinyl]methyl]acetamide.

To a solution of 5-formyl furyl derivative (140 mg 0.31 mmol) in dry pyridine was added hydroxylamine hydrochloride (26 mg, 0.38 mmol). The whole reaction mixture was stirred at 25° C. for 4.0 hr. TLC of the reaction Mixture was monitored. A slower moving spot was observed compare to starting compound. Pyridine was removed under reduced pressure and traces of pyridine were removed with toluene to yield title compound of 140 mg.

δppm ¹H NMR (DMSO-d₆): 8.70(d, 2H), 8.08-8.03(m, 1H), 7.65-7.61 (m,1H), 7.78 (d,1H), 7.24 7.11 (m, 2H), 4.70 (d,1H) 4.49 (s, 2H), 4.07 (t,1H), 1.82 (s, 3H), 3.72 (m, 2H), 3.53-2.88 (m, 9H).

Compound No. 61: (S)-N-[[3-[3-Fluoro-4-[N-1[4-{2-furyl(5-aldoxime(methyl-4-(N-carboxyaminophenylacetate) methyl}]piperazinyl]phenyl]-2-oxo-5-oxazolidinyl]methyl]acetamide

The title compound was prepared by using the procedure mentioned for Compound No. 60.

Compound No. 62: (S)-N-[[3-[3-Fluoro-4[N-1-[4-{2-furyl-(5-hydrazone)-methyl}]-piperazinyl]-phenyl]-2-oxo-5-oxazolidinyl]-methyl]acetamide

To a solution of 5-formyl furyl derivative (140 mg, 0.31 mmol) in ethanol (4.0 ml) was added hydrazine hydrate (100 mg) and catalytic amount of conc. sulfuric acid. The whole reaction mixture was stirred at 25° C. for 48 hr. TLC of the reaction mixture showed no changes. Stirring was continued for another 12 hr. No change in TLC was observed.

Solvent was evaporated to dryness and the solid residue was digested with ether to give 100 mg of title compound of m.p. 178-181° C.

δppm (CDCl₃):δ=7.61 (s, 1H), 7.42 (dd,1H), 7.04 (t,1H), 6.92 (t,1H), 6.44 (d,1H), 6.28 (bs, 2H), 5.60 (bs, 2H), 4.77 (bs, 1H), 4.02 (t,1H), 3.77-3.61 (m, 8H), 3.10 (bs, 1H), 2.71 (bs, 1H), 2.02 (s, 3H).

Compound No. 63: Preparation of (S)-N-[[3-[3-Fluoro-4-[N-1{2-furyl-[4-(5-hydroxymethyl)methyl)}]piperazinyl]-2-oxo-5-oxazolidinyl]methyl]acetamide

To a solution of 5-formyl-2-derivative (100 mg, 0.22 mmol) in ethanol was added Sodium borohydride (solid, 17 mg, 0.44 mmol). The whole reaction mixture was stirred at 25° C. for 60 hr. TLC of the reaction mixture in chloroform:Methanol (9:1) showed a slower moving spot. The solvent was removed under reduced pressure. The residue was dissolved in chloroform and washed with water, dried over anhydrous sodium sulphate and solvent was removed to give title compound in 70 mg as gum.

δppm CDCl₃) : 745 (d,1H), 7.06 (d,1H), 6.94 (d, 1H), 6.23 (dd,1H), 6.00 (bs, 1H), 4.70 (bs, 1H), 4.03 (t, 1H), 3.12 (bs, 4H), 2.69 (bs, 4H), 4.62 (s, 2H), 3.76-3.4 (m, 6H), 2.03 (s, 3H).

Compound No. 64: (S)-N-[[3-[3-Fluoro-4-[N-1[4-{2-furyl(5-cyano)methyl}]piperazinyl]phenyl]-2-oxo-5-oxazolidinyl]methyl]acetamide

(S)-N-[[3-[3-Fluoro-4-[N-1[4-{2-furyl(5-aldoxime)methyl }]piperazinyl]phenyl]-2-oxo-5-oxazolidinyl]methyl]acetamide (6126, 3.5 g, 0.76 mmol) was taken in CH₂Cl₂ (5 mL) and triethyl amine(1.5 g, 1.5 mmol) was added and the reaction mixture was maintained at −78° C. Triflic anhydride (4.3 g, 1.5 mmol) in CH₂Cl₂ (2 mL) was added dropwise after complete addition, the temperature of the reaction mixture was allowed to rise to r.t. in 2 hrs. The r.m. is concentrated under vacuum. H₂O (10 mL) was added and extracted with CH₂Cl₂ (3×10 mL). The combined organic layer was dried over Na₂SO₄, filtered and concentrated to obtain the title compound.

NMR(CDCl₃); 7.44-6.10(m, 6H), 4.74(m, 1H), 4.00(t, 2H), 3.73-3.62(m, 5H), 3.09-2.68 (m, 8H, ), 2.01(s, 3H)

Compound No. 65: (S)-N-[[3-Fluoro-4-[N-1[4-{2-furyl(5-carboxy)methyl}]-piperazinyl]phenyl]-2-oxo-5-oxazolidinyl]methyl]acetamide

The title compound was made using (S)-N-[[3-Fluoro-4-[N-1[4-{2-furyl(5-formyl)methyl }]piperazinyl]phenyl]-2-oxo-5-oxazolidinyl]methyl]acetamide in a solution of freshly prepared Ag₂O and stirring for 30 min. The r.m. was filtered, acidified to pH 5 and extracted with EtOAc, dried over Na₂SO₄, filtered and concentrated.

δppm CDCl₃₊ MeOD) 8.01-7.03 (m, 5H), 4.81 (m, 1H), 4.07 (t, 1H), 3.8-3.3 (m, 5H), 3.0(s, 4H), 2.7 (s, 4H) 2.01(s, 3H).

Compound No. 66: (S)-N-[[3-Fluoro-4-[N-1[5-(1,3-dioxane)-2-furylmethyl]piperazinyl]phenyl]-2-oxo-5-oxazolidinyl]methyl]acetamide

The title compound was made using (S)-N-[[3-Fluoro-4-[N-1[4-{2-furyl(5-formyl)methyl }]piperazinyl]phenyl]-2-oxo-5-oxazolidinyl]methyl]acetamide with 1,3-propane diol and BF₃ etherate using standard literature procedures.

Compound No. 67: (S)-N-[[3-Fluoro-4-[N-1[5-(formamido)-2-furylmethyl]-piperazinyl]phenyl]-2-oxo-5-oxazolidinyl]methyl]acetamide

The title compound was made reacting (S)-N-[[3-Fluoro-4-[N-1[4-(2-furyl-(5-carboxyethyl)methyl)piperazinyl]phenyl]-2-oxo-5-oxazolidinyl]methyl]acetamide with aqueous ammonia solution followed by wet extraction with ethyl acetate.

δppm CDCl₃₊ DMSO-d6) 7.46-6.37 (m, 6H), 4.7 (m, 1H), 4.0-3.4 (m, 5H), 2.9 (s, 4H), 2.4 (s, 4H ), 2.01 (s, 3H).

Compound No. 68: (S)-N-[[3-Fluoro-4-[N-1[5-(morpholine-1-carbonyl)-2-furyl-methyl]piperazinyl]phenyl]-2-oxo-5-oxazolidinyl]methyl]acetamide

The title compound was made by reacting (S)-N-[[3-Fluoro-4-[N-1[4-(2-furyl-(5-carboxyethyl)methyl)piperazinyl]phenyl]-2-oxo-5-oxazolidinyl]methyl]acetamide with morpholine using standard literature procedure.

Compound No. 69: (S)-N-[[3-Fluoro-4-[N-1[5-(4-(tert butoxy carbonyl)amino piperidine)-2-furylmethyl]piperazinyl]phenyl]-2-oxo-5-oxazolidinyl]methyl]acetamide

The title compound was made by reacting (S)-N-[[3-Fluoro-4-[N-1[4-(2-furyl-(5-carboxy)methyl)piperazinyl]phenyl]-2-oxo-5-oxazolidinyl]methyl]acetamide with thionyl chloride and 4-(tert butoxy carbonyl)amino piperidine.

While the present invention has been described in terms of its specific embodiments, certain modifications and equivalents will be apparent to those skilled in the art and are intended to be included within the scope of the present invention.

1. A compound having the structure of Formula I 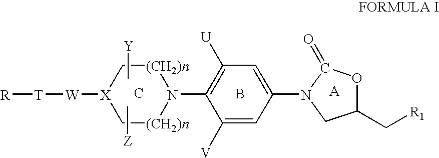
and its pharmaceutically acceptable salts, enantiomers, diastearomers, N-oxides, prodrugs or metabolites, wherein
T is five to seven membered heterocyclic ring, aryl, substituted aryl, bound to the ring C with a linker W and the heterocyclic and aryl rings are further substituted by a group represented by R,
wherein R is selected from the group consisting of alkyl (C₁-C₆), halogen, —CN, COR₅, COOR₅, N(R₆,R₇), CON (R₆, R₇), CH₂NO₂, NO₂, CH₂R₈, CHR₉, —CH═N—OR₁₀, —C═CH—R₅, wherein R₅ is selected from the group consisting of H, optionally substituted C₁-C₁₂, alkyl, C₃₋₁₂, cycloalkyl, aryl, heteroaryl; R₆ and R₇ are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl, F, Cl, Br, C₁₋₁₂ alkyl substituted with one or more of F, Cl, Br, I, OR₄, SR₄, N(R₆,R₇) wherein R₄ is selected from the group consisting of H, C₁₋₁₂ alkyl, C₃₋₁₂ cycloalkyl, C₁₋₆ alkoxy, C₁₋₆ alkyl substituted with one or more F, Cl, Br, I or OH and R₆ and R₇ are the same as defined earlier, R₁₀ is selected from the group consisting of H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3;
X is CH, CH—S, CH—O and N;
Y and Z are independently selected from the group consisting of hydrogen, C₁₋₆ alkyl, C₃₋₁₂ cycloalkyl, C₀₋₃ bridging group;
U and V are independently selected from the group consisting of optionally substituted C₁₋₆ alkyl , F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁₁)CH₂—, —CO—CO—, CH₂ (R₁₁)N—, CH (R₁₁), S, CH₂(CO), N(R₁₁) wherein R₁₁ is hydrogen, optionally substituted with C₁₋₁₂ alkyl, C₃₋₁₂ cycloalkyl, C₁₋₆ alkoxy, C₁₋₆ alkyl , aryl , heteroaryl; and
R₁ is selected from the group consisting of —NHC(═O)R₂ wherein R₂ is hydrogen , C₁₋₁₂ alkyl, C₃₋₁₂ cycloalkyl, C₁₋₆ alkoxy, C₁₋₆ alkyl substituted with one or more of F, Cl, Br, I or OH; N(R₃, R₄); —NR₂C(═S) R₃: —NR₂C(═S)SR₃ wherein R₂ is the same as defined above and R₃ and R₄ are independently selected from the group consisting of H, C₁₋₁₂ alkyl, C₃₋₁₂ cycloalkyl, C₁₋₆ alkoxy, C₁₋₆ alkyl substituted with one or more of F, Cl, Br, I or OH.

2. A compound having structure of Formula II 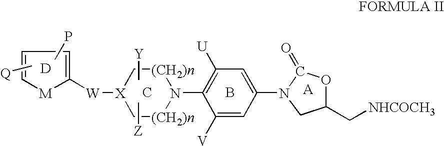
and its pharmaceutically acceptable salts, enantiomers, diastearomers, N-oxides, prodrugs or metabolites wherein
M is O,S, NH, or NCH₃;
X is CH, CH—S, CH—O and N;
Y and Z are independently selected from the group consisting of hydrogen, C₁₋₆ alkyl, C₃₋₁₂ cycloalkyl, C₀₋₃ bridging group;
U and V are independently selected from the group consisting of optionally substituted C₁₋₆ alkyl, F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CO—CO—, —CH₂NHCH₂, —CH₂—N (R₁₁)CH₂—, CH₂ (R₁₁)N—, CH (R₁₁) , S, CH₂(CO), N(R₁₁) wherein R₁₁ is hydrogen, optionally substituted with C₁₋₁₂ alkyl, C₃₋₁₂ cycloalkyl, C₁₋₆ alkoxy, C₁₋₆ alkyl , aryl , heteroaryl except when M=S, Q=P═H, W═(C═O);
n is an integer in the range from 0 to 3; and,
Q and P are independently selected from the group consisting of hydrogen, —CN, COR₅, COOR₅, N (R₆, R₇), CON (R₆,R₇), CH₂NO₂, NO₂, CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, heteroaryl; R₆ and R₇ are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl ,F, Cl, Br, C₁₋₁₂ alkyl substituted with one or more of F, Cl, Br, I, OR₄, SR₄, wherein R₄ is selected from the group consisting of H, C₁₋₁₂ alkyl, C₃₋₁₂ cycloalkyl, C₁₋₆ alkoxy, C₁₋₆ alkyl substituted with one or more F, Cl, Br, I or OH, N(R₆, R₇), R₁₀ is selected from the group consisting of H, optionally substituted C₁₋₁₂ alkyl, C₃₋₁₂ cycloalkyl, C₁₋₆ alkoxy, C₁₋₆ alkyl , aryl , heteroaryl except W═(CO), Q and P═H and M=S, ring C in Formula II is 6-8 membered or of larger size and the larger rings have either two or three carbons between each nitrogen atom, comprising of 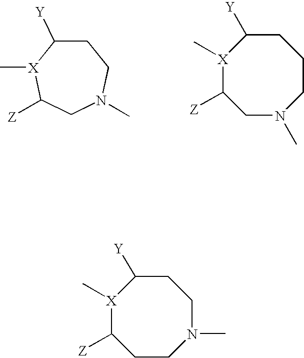
and may be bridged to form a bicyclic system as shown below, 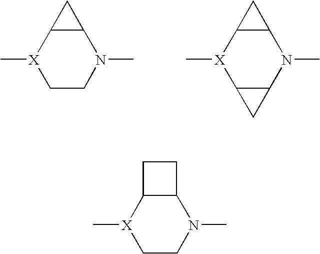
ring C is optionally substituted by Y and Z with alkyl groups, cycloalkyl groups, fluoro group, carboxylic and corresponding esters, amides, substituted alkyls or bridging alkyl groups are as shown below: 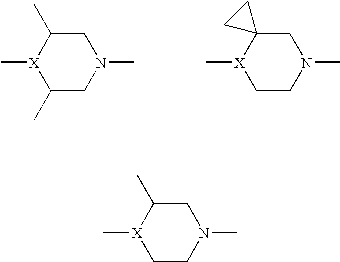
six membered ring C with X=—CH—(NR₁₁), (wherein R₁₁ is the same as defined earlier) is selected from the group consisting of the following rings;

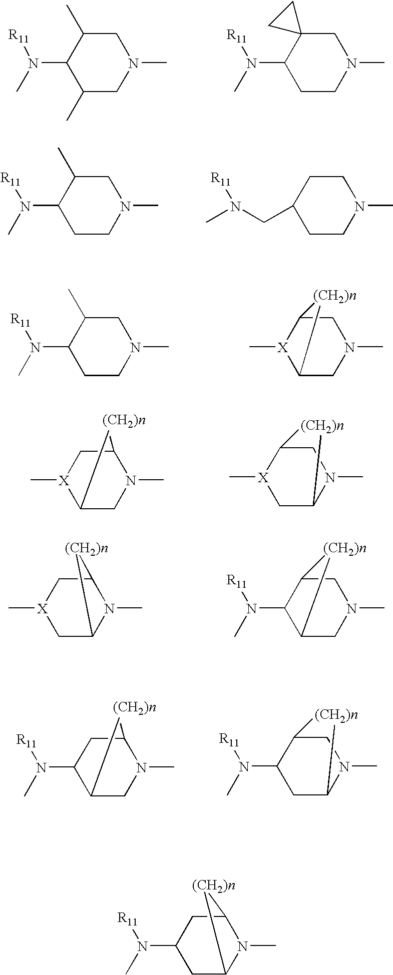

3. The compound of claim 2 having the structure of Formula III, when M=S 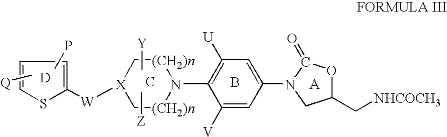
wherein P, Q, U, V, X, Y, Z, W and n in Formula III are as defined earlier for Formula II.

4. The compound of claim 2 having the structure of Formula IV, when M=O 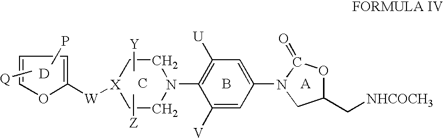
wherein P, Q, U, V, X, Y, Z, W and n in Formula IV are as defined earlier for Formula II.

5. Compound selected from the group consisting of
1. (S)-N-[[3-[3-Fluoro-4-[N-1-[4-(2-furoyl) piperazinyl]]phenyl]-2-oxo-5-oxazolidinyl]methyl]acetamide
2. (S)-N-[[3-[3-Fluoro-4-[N-1[4-{2-furyl(5-formyl)methyl}]piperazinyl]phenyl]-2-oxo-5-oxazolidinyl]methyl]acetamide
3. (S)-N-[[3-Fluoro-4-[N-1[4-(2-furyl-(5-carboxyethyl)methyl)piperazinyl]phenyl]-2-oxo-5-oxazolidinyl]methyl]acetamide
4. (S)-N-[[3-Fluoro-4-[N-1[4-(5-bromo-2-furoyl)]piperazinyl]phenyl]-2-oxo-5-oxazolidinyl]methyl]acetamide
5. (S)-N-[[3-Fluoro-4-[N-1[4-(5-chloromethyl-2-furoyl)piperazinyl]phenyl]-2-oxo-5-oxazolidinyl]methyl]acetamide
6. (S)-N-[[3-Fluoro-4-[N-1[4-(5-nitro-2-furoyl)piperazinyl]phenyl]-2-oxo-5-oxazolidinyl]methyl]acetamide
7. (S)-N[[3-[3-Fluoro-4-[N-1[4-{2-(2-thienyl)dicarbonyl}]piperazinyl]phenyl]2-oxo-5-oxazolidinyl]methyl]acetamide
8. (S)-N[[3-[3-Fluoro-4-[N-1[4-(3-furoyl)]piperazinyl]phenyl]2-oxo-5-oxazolidinyl]methyl]acetamide
9. (S)-N[[3-[3-Fluoro-4-[N-1[4-{2-furyl(5-bromo)methyl}]piperazinyl]phenyl]2-oxo-5-oxazolidinyl]methyl]acetamide
10. (S)-N[[3-[3-Fluoro-4-[N-1[4-{2-thienyl(5-chloro)methyl}]piperazinyl]phenyl]2-oxo-5-oxazolidinyl]methyl]acetamide
11. (S)-N[[3-[3-Fluoro-4-[N-1[4-(2-furylmethyl)]piperazinyl]phenyl]2-oxo-5-oxazolidinyl]methyl]acetamide
12. (S)-N-[[3-[3-Fluoro-4-[N-1[4-(2-thienylmethyl)]piperazinyl]phenyl]-2-oxo-5-oxazolidinyl]methyl]acetamide
13. (S)-N[[3-[3-Fluoro-4-[N-1[4-(2-thienylacetyl)]piperazinyl]phenyl]2-oxo-5-oxazolidinyl]methyl]acetamide
14. (S)-N-[[3-[3-Fluoro-4-[N-1[4-{2-thienyl(4-bromo)methyl}]piperazinyl]phenyl]-2oxo-5-oxazolidinyl]methyl]acetamide
15. (S)-N-[[3-[3-fluoro-4-[N-1-[4-{2-furyl-(5-nitro)methyl}]piperazinyl]phenyl]-2-oxo-5-oxazolidinyl]methyl]acetamide.
16. Hydrochloride salt of (S)-N-[[3-Fluoro-4-[N-1[4-{2-furyl(5-nitro)methyl}]piperazinyl]phenyl]-2-oxo-5-oxazolidinyl]methyl]acetamide
17. Citrate salt of (S)-N-[[3-Fluoro-4-[N-1[4-{2-furyl(5-nitro)methyl}]piperazinyl]phenyl]-2-oxo-5-oxazolidinyl]methyl]acetamide
18. (S)-N[[3-[3-Fluoro-4-[N-1[4-(2-pyrrolylmethyl)]piperazinyl]phenyl]2-oxo-5-oxazolidinyl]methyl]acetamide
19. (S)-N[[3-[3-Fluoro-4-[N-1[4-{2-thienyl(3-methyl)methyl}]piperazinyl]phenyl]2-oxo-5-oxazolidinyl]methyl]acetamide
20. (S)-N[[3-[3-Fluoro-4-[N-1[4-(3-furylmethyl)]piperazinyl]phenyl]2-oxo-5-oxazolidinyl]methyl]acetamide
21. (S)-N[[3-[3-Fluoro-4-[N-1[4-{2-thienyl(5-methyl)methyl}]piperazinyl]phenyl]2-oxo-5-oxazolidinyl]methyl]acetamide
22. (S)-N[[3-[3-Fluoro-4-[N-1[4-{(2-pyrrole(1-methyl)methyl}]piperazinyl]phenyl]2-oxo-5-oxazolidinyl]methyl]acetamide
23. (S)-N[[3-[3-Fluoro-4-[N-1[4-{2-thienyl(5-nitro)methyl}]piperazinyl]phenyl]2-oxo-5-oxazolidinyl]methyl]acetamide
24. (S)-N[[3-[3-Fluoro-4-[N-1[4-[2-furyl{5-(N-thiomorpholinyl)methyl}methyl]piperazinyl]phenyl]2-oxo-5-oxazolidinyl]methyl]acetamide
25. (S)-N[[3-[3-Fluoro-4-[N-1[4-[2-furyl{5-(N-morpholinyl)methyl )methyl]]piperazinyl]phenyl]2-oxo-5-oxazolidinyl]methyl]acetamide
26. (S)-N-[[3-Fluoro-4-[N-1[4-(2-furyl(5-acetoxymethyl)methyl}]piperazinyl]phenyl]-2-oxo-5-oxazolidinyl]methyl]acetamide
27. (S)-N-[[3-Fluoro-4-[N-1[4-{2-thienyl(5-bromo)methyl}]piperazinyl]phenyl)-2-oxo-5-oxazolidinyl]methyl]acetamide
28. (S)-N-[[3-Fluoro-4-[N-1[4-(5-nitro-2-furylmethyl)piperazinyl]phenyl]-2-oxo oxazolidinyl]methyl]dichloroacetamide
29. (S)-N[[3-[3-Fluoro-4-[N-1[4-(5-nitro-2-thienoyl)]piperazinyl]phenyl]2-oxo-5-oxazolidinyl]methyl]acetamide hydrochloride
30. (S)-N[[3-[3-Fluoro-4-[N-1[4-(2′,2′-diphenyl-2′hydroxy acetyl)]piperazinyl]phenyl]2-oxo-5-oxazolidinyl]methyl]acetamide
31. (S)-N-[[3-[3-Fluoro[4-[3-(1α,5α,6α)-6-[N-(5-nitro-2-furoyl )-N-methyl]amino]-3-azabicyclo-[3.1.0]hexane]phenyl]-2-oxo-5-oxazolidinyl]methyl]acetamide
32. (S)-N-[[3-[3-Fluoro[4-[3-(1α,5α,6α)-6-[N-(3-furoyl)-N-methyl]amino]-3-azabicyclo-[3.1.0]hexane]phenyl]-2-oxo-5-oxazolidinyl]methyl]acetamide
33. (S)-N-[[3-[3-Fluoro[4-3-(1α,5α,6α)-6-[N-(5-bromo-2-furoyl)-N-ethyl]amino]-3-azabicyclo-[3.1.0]hexane]phenyl]-2-oxo-5-oxazolidinyl]methyl]acetamide
34. (S)-N-[[3-[3-Fluoro[4-[3-(1α,5α,6α)-6-[N-(5-nitro-2-thienylmethyl)-N-methyl]amino]-3-azabicyclo-[3.1.0]hexane]phenyl]-2-oxo-5-oxazolidinyl]methyl]acetamide
35. (S)-N-[[3-[3-Fluoro[4-[3-(1α,5α,6α)-6-[N-(5-nitro-2-furylmethyl)-N-methyl]amino]-3-azabicyclo-[3.1.0]hexane]phenyl]-2-oxo-5-oxazolidinyl]methyl]acetamide
36. (S)-N-[[3-[3-Fluoro[4-[3-(1α,5α,6α)-6-[N-(5-formyl-2-furylmethyl)-N-methyl]amino-methyl]-3-azabicyclo-[3.1.0]hexane]phenyl]-2-oxo-5-oxazolidinyl]methyl]acetamide
37. (S)-N-[[3-[3-Fluoro[4-[3-(1α,5α,6α)-6-[N-(5-carboxyethyl-1-2-furylmethyl)-N-methyl]aminomethyl]-3-azabicyclo-[3.1.0]hexane]phenyl]-2-oxo-5-oxazolidinyl]methyl]acetamide
38. (S)-N-[[3-[3-Fluoro[4-[3-(1α,5α,6α)-6-[N-(2-thiopheneacetyl)-N-methyl]aminomethyl]-3-azabicyclo-[3.1.0]hexane]phenyl]-2-oxo-5-oxazolidinyl]methyl]acetamide
39. (S)-N-[[3-[3-Fluoro[4-[3-(1α,5α,6α)-6-[N-(5-nitro-2-thienylmethyl)-N-methyl]amino-methyl]-3-azabicyclo-[3.1.0]hexane]phenyl]-2-oxo-5-oxazolidinyl]methyl]acetamide
40. (S)-N-[[3-[3-Fluoro[4-[3-(1α,5α,6α)-6-[N-(5-nitro-2-furylmethyl)-N-methyl]aminomethyl]-3-azabicyclo-[3.1.0]hexane]phenyl]-2-oxo-5-oxazolidinyl]methyl]acetamide
41. (S)-N-[[3-[4-[4-(N-methyl-N-2furyl(5formyl)methylaminopiperidine-1-yl]-3-fluorophenyl]-2-oxo-oxazolidin-5-yl]methyl]acetamide
42. (S)-N-[[3-[4-[4-(N-methyl-N-(3,5-difluorobenzoyl)aminopiperidine-1-yl]-3-fluorophenyl]-2-oxo-oxazolidin-5-yl]methyl]acetamide.
43. (S)-N-[[3-[4-[4-(N-methyl-N-(5-bromo-2-furoyl)aminopiperidine-1-yl]-3-fluorophenyl]-2-oxo-oxazolidin-5-yl]methyl]acetamide
44. (S)-N-[[3-[4-[4-(N-methyl-N-(5-nitro-2-furoyl)aminopiperidine-1-yl]-3-fluorophenyl]-2-oxo-oxazolidin-5-yl]methyl]acetamide
45. (S)-N-[[3-[4-[4-(N-methyl-N-3-furoyl)aminopiperidine-1-yl]-3-fluorophenyl]-2-oxo-oxazolidin-5-yl]methyl]acetamide.
46. (S)-N-{3-[4-[4-(N-methyl, N-2-furoyl )aminopiperidine-1-yl]-3-fluorophenyl]-2-oxo-oxazolidin-5-yl methyl]acetamide
47. (S)-N-{3-[4-[4-(N-methyl,2-thiopheneacetyl)aminopiperidine-1-yl]-3-fluorophenyl]-2-oxo oxazolidin-5-yl methyl]acetamide
48. (S)-N-[[3-[4-[4-(N-methyl-N-2furylmethyl)aminopiperidine-1-yl]-3-fluorophenyl]-2-oxo-oxazolidin-5-yl]methyl]acetamide
49. (S)-N-[[3-[4-[4-(N-methyl-N-3-furyl)aminopiperidine-1-yl]-3-fluorophenyl]-2-oxo-oxazolidin-5-yl]methyl]acetamide.
50. (S)-N-[[3-[4-[4-(N-methyl-N-2-furyl(5-nitro)methyl)aminopiperidine-1-yl]-3-fluorophenyl]-2-oxo-oxazolidin-5-yl]methyl]acetamide.
51. (S)-N-[[3-[4-[4-(N-methyl-N-2-thienyl(5-nitro)methyl)aminopiperidine-1-yl]-3-fluorophenyl]-2-oxo-oxazolidin-5-yl]methyl]acetamide.
52. (S)-N-[[3-[4-[4-(N-methyl-N-2-thienylmethyl)aminopiperidine-1-yl]-3-fluorophenyl]-2-oxo-oxazolidin-5-yl]methyl]acetamide.
53. (S)-N-[[3-[4-[4-(N-methyl-N-(5-methyl-2-thienylmethyl)aminopiperidine-1-yl]-3-fluorophenyl]-2-oxo-oxazolidin-5-yl]methyl]acetamide
54. (S)-N-(3-[4-[4-(N-methyl,2-(5-bromo)thienylmethyl)aminopiperidine-1-yl]-3-fluorophenyl]-2-oxo-oxazolidin-5-yl methyl]acetamide
55. (S)-N[[3-[3-Fluoro-4-[N-1[4-{2-furyl(5-formyl)methyl}]homopiperazinyl]phenyl]2-oxo-5-oxazolidinyl]methyl]acetamide
56. (S)-N[[3-[3-Fluoro-4-[N-1[4-(2-thienylacetyl)]homopiperazinyl]phenyl]2-oxo-5-oxazolidinyl]methyl]acetamide
57. (S)-N[[3-[3-Fluoro-4-[N-1[4-{2-thienyl(5-nitro)methyl}]homopiperazinyl]phenyl]2-oxo-5-oxazolidinyl]methyl]acetamide
58. (S)-N[[3-[3-Fluoro-4-[N-1[4-(3-furylmethyl)]homopiperazinyl]phenyl]2-oxo-5-oxazolidinyl]methyl]acetamide
59. Preparation of (S)-N-[[3-[3-fluoro-4-[N-1(2-furyl-[4-(5-difluoromethyl)methyl }]piperazinyl]-2-oxo-5-oxazolidinyl]-methyl]acetamide.
60. (S)-N-[[3-[3-Fluoro-4-[N-1-[4-(2-furyl-(5-aldoxime)methyl}]piperazinyl]phenyl]-2-oxo-5-oxazolidinyl]methyl]acetamide
61. (S)-N-[[3-[3-Fluoro-4-[N-1[4-{2-furyl(5-aldoxime(methyl-4-(N-carboxyaminophenyl acetate)methyl}]piperazinyl]phenyl]-2-oxo-5-oxazolidinyl]methyl]acetamide
62. (S)-N-[[3-[3-Fluoro-4[N-1-[4-{2-furyl-(5-hydrazone)-methyl}]-piperazinyl]-phenyl]-2-oxo-5-oxazolidinyl]-methyl]acetamide
63. Preparation of (S)-N-[[3-[3-Fluoro-4-[N-1{2-furyl-[4-(5-hydroxymethyl) methyl}]piperazinyl]-2-oxo-5-oxazolidinyl]methyl]acetamide
64. (S)-N-[[3-[3-Fluoro-4-[N-1[4-{2-furyl(5-cyano)methyl}]piperazinyl]phenyl]-2-oxo-5-oxazolidinyl]methyl]acetamide
65. (S)-N-[[3-Fluoro-4-[N-1[4-{2-furyl(5-carboxy)methyl}]piperazinyl]phenyl]-2-oxo-5-oxazolidinyl]methyl]acetamide
66. (S)-N-[[3-Fluoro-4-[N-1[5-(1,3-dioxane)-2-furylmethyl]piperazinyl]phenyl]-2-oxo-5-oxazolidinyl]methyl]acetamide
67. (S)-N-[[3-Fluoro-4-[N-1[5-(formamido)-2-furylmethyl]piperazinyl]phenyl]-2-oxo-5-oxazolidinyl]methyl]acetamide
68. (S)-N-[[3-Fluoro-4-[N-1[5-(morpholine-1-carbonyl)-2-furylmethyl]piperazinyl]phenyl]-2-oxo-5-oxazolidinyl]methyl]acetamide
69. (S)-N-[[3-Fluoro-4-[N-1[5-(4-(tert butoxy carbonyl)amino piperidine)-2-furylmethyl]piperazinyl]phenyl]-2-oxo-5-oxazolidinyl]methyl]acetamide
70. (S)-N-[[3-Fluoro-4-[N-1[4-{(Z)-2-methoxyimino-2-(2-furyl)acetyl}]piperazinyl]phenyl]-2-oxo-5-oxazolidinyl]methyl]acetamide
71. (S)-N-[[3-[3-Fluoro[4-[3-(1α,5α,6α)-6-[N-(2-thiopheneacetyl)-N-methyl]amino]-3-azabicyclo-[3.1.0]hexane]phenyl]-2-oxo-5-oxazolidinyl]methyl]acetamide
72. (S)-N-[[3-[3-Fluoro[4-[3-(1α,5α,6α)-6-[N-(5-formyl-2-furylmethyl)-N-methyl]amino]-3-azabicyclo-[3.1.0]hexane]phenyl]-2-oxo-5-oxazolidinyl]methyl]acetamide
73. (S)-N-[[3-[3-Fluoro[4-[3-(1α,5α,6α)-6-[N-(3-thienoyl)-N-methyl]amino]-3-azabicyclo[3.1.0]hexane]phenyl]-2-oxo-5-oxazolidinyl]methyl]acetamide
74. (S)-N-[[3-[3-fluoro-4-[N-1{2-furyl-[4-(5-fluoromethyl) methyl}]piperazinyl]-2-oxo-5-oxazolidinyl]-methyl]acetamide.

6. A pharmaceutical composition comprising the compound of claims 1, 2, or 5 and a pharmaceutical acceptable carrier.

7. A pharmaceutical composition comprising a pharmaceutically effective amount of compound according to claims 1, 2, or 5, or a physiologically acceptable acid addition salt thereof with a pharmaceutical acceptable carrier for treating microbial infections.

8. A method of treating or preventing microbial infections in a mammal comprising administering to the said mammal, the pharmaceutical composition according to claim 7.

9. The method according to claim 8 wherein the microbial infections are caused by gram-positive and gram-negative bacteria.

10. The method according to claim 9 wherein gram-positive bacteria are selected from the group consisting of staphylococcus spp., streptococcus spp. (including pneumoniae cocci), enterococcus spp., bacillus spp., corynebacterium spp., clostridia spp., peptostreptococcus spp., listeria spp. and legionella spp.

11. A method of treating or preventing aerobic and anaerobic bacterial infections in a mammal comprising administering to said mammal, a therapeutically effective amount of a compound having the structure of Formula I 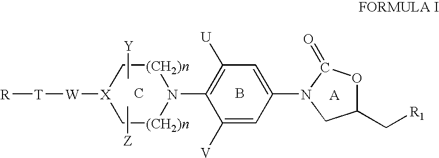
or its pharmaceutically acceptable salts, enantiomers, diastearomers, N-oxides, prodrugs or metabolites, wherein
T is five to seven membered heterocyclic ring, aryl, substituted aryl, bound to the ring C with a linker W and the heterocyclic and aryl rings are further substituted by a group represented by R,
wherein R is selected from the group consisting of alkyl (C₁-C₆), halogen, —CN, COR₅,COOR₅, N(R₆, R₇), CON (R₆, R₇), CH₂NO₂, NO₂, CH₂R₈, CHR₉, —CH═N—OR₁₀, —C═CH—R₅, wherein R₅ is selected from the group consisting of H, optionally substituted C₁-C₁₂, alkyl, C₃₋₁₂, cycloalkyl, aryl, heteroaryl; R₆ and R₇ are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl, F, Cl, Br, C₁₋₁₂ alkyl substituted with one or more of F, Cl, Br, I, OR₄, SR₄, N(R₆,R₇) wherein R₄ is selected from the group consisting of H, C₁₋₁₂ alkyl, C₃₋₁₂ cycloalkyl, C₁₋₆ alkoxy, C₁₋₆ alkyl substituted with one or more F, Cl, Br, I or OH and R₆ and R₇ are the same as defined earlier, R₁₀ is selected from the group consisting of H, optionally substituted C₁₋₁₂ alkyl, C₃₋₁₂ cycloalkyl, C₁₋₆, alkoxy, C₁₋₆ alkyl, aryl, heteroaryl;
n is an integer in the range from 0 to 3;
X is CH, CH—S, CH—O and N;
Y and Z are independently selected from the group consisting of hydrogen, C₁₋₆ alkyl, C₃₋₁₂cycloalkyl, C₀₋₃ bridging group;
U and V are independently selected from the group consisting of optionally substituted C₁₋₆ alkyl , F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁₁)CH₂—, —CO—CO—, CH₂ (R₁₁)N—, CH (R₁₁), S, CH₂(CO), N(R₁₁) wherein R₁₁ is optionally substituted with C₁₋₁₂ alkyl, C₃₋₁₂ cycloalkyl, C₁₋₆ alkoxy, C₁₋₆ alkyl , aryl , heteroaryl; and
R₁ is selected from the group consisting of —NHC(═O)R₂ wherein R₂ is hydrogen, C₁₋₁₂ alkyl, C₃₋₁₂ cycloalkyl, C₁₋₆ alkoxy, C₁₋₆ alkyl substituted with one or more of F, Cl, Br, I or OH; N(R₃, R₄); —NR₂C(═S) R₃: —NR₂C(═S)SR₃ wherein R₂ is the same as defined above and R₃ and R₄ are independently selected from the group consisting of H, C₁₋₁₂ alkyl, C₃₋₁₂ cycloalkyl, C₁₋₆ alkoxy, C₁₋₆ alkyl substituted with one or more of F, Cl, Br, I or OH.

12. A method of treating or preventing aerobic and anaerobic bacterial infections in a mammal comprising administering to said mammal, a therapeutically effective amount of a compound having the structure of Formula II 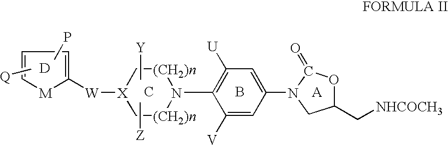
or its pharmaceutically acceptable salts, enantiomers, diastearomers, N-oxides, prodrugs or metabolites wherein
M=O, S, NH or N—CH₃,
X is CH, CH—S, CH—O and N;
Y and Z are independently selected from the group consisting of hydrogen, C₁₋₆ alkyl, C₃₋₁₂ cycloalkyl, C₀₋₃ bridging group;
U and V are independently selected from the group consisting of optionally substituted C₁₋₆ alkyl, F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁₁)CH₂—, CH₂ (R₁₁)N—, —CO—CO—, CH (R₁₁), S, CH₂(CO), N(R₁₁) wherein R₁₁ is hydrogen, optionally substituted with C₁₋₁₂ alkyl, C₃₋₁₂ cycloalkyl, C₁₋₆ alkoxy, C₁₋₆ alkyl , aryl , heteroaryl except when M=S, Q=P=H, W=(C═O);
n is an integer in the range from 0 to 3; and,
Q and P are independently selected from the group consisting of hydrogen, —CN, COR₅, COOR₅, N (R₆, R₇), CON (R₆,R₇), CH₂NO₂, NO₂,CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, heteroaryl; R₆ and R₇ are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl ,F, Cl, Br, C₁₋₁₂ alkyl substituted with one or more of F, Cl, Br, I, OR₄, SR₄, wherein R₄ is selected from the group consisting of H, C₁₋₁₂ alkyl, C₃₋₁₂ cycloalkyl, C₁₋₆ alkoxy, C₁₋₆ alkyl substituted with one or more F, Cl, Br, I or OH, N(R₆, R₇), R₁₀ is selected from the group consisting of H, optionally substituted C₁₋₁₂ alkyl, C₃₋₁₂ cycloalkyl, C₁₋₆ alkoxy, C₁₋₆ alkyl , aryl , heteroaryl except W=(CO), Q and P=H and M=S, ring C in Formula II is 6-8 membered or of larger size and the larger rings have either two or three carbons between each nitrogen atom, comprising of 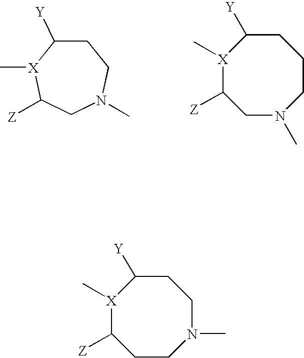
and may be bridged to form a bicyclic system as shown below, 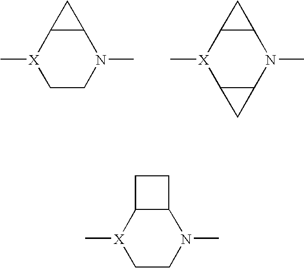
ring C is optionally substituted by Y and Z with alkyl groups, cycloalkyl groups, fluoro group, carboxylic and corresponding esters, amides, substituted alkyls or bridging alkyl groups are as shown below: 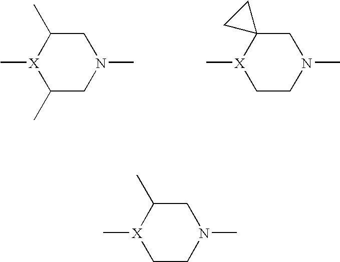
six membered ring C with X=—CH—(NR₁₁), (wherein R₁₁ is the same as defined earlier) is selected from the group consisting of the following rings;

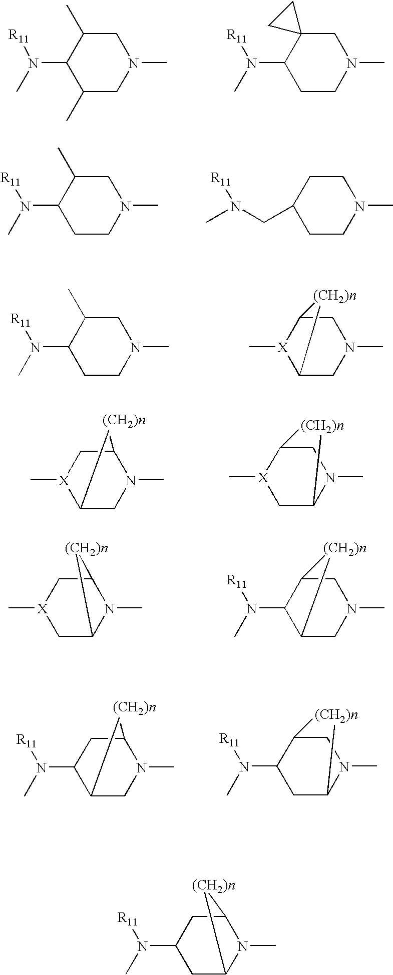

13. A method of treating or preventing aerobic and anaerobic bacterial infections in a mammal comprising administering to said mammal, a therapeutically effective amount of a compound namely, (S)-N-[[3-Fluoro-4-[N-1[4-{2-furyl(5-nitro)methyl}]piperazinyl]phenyl]-2-oxo-5-oxazolidinyl]methyl]acetamide hydrochloride.

14. A method of treating or preventing catheter infections and foreign body or prosthesis infections in a mammal comprising administering to said mammal, a therapeutically effective amount of a compound having the structure of Formula I 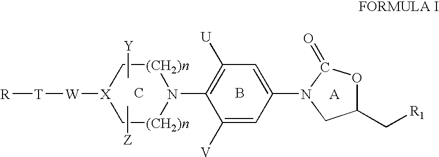
or its pharmaceutically acceptable salts, enantiomers, diastearomers, N-oxides, prodrugs or metabolites, wherein
T is five to seven membered heterocyclic ring, aryl, substituted aryl, bound to the ring C with a linker W and the heterocyclic and aryl rings are further substituted by a group represented by R,
wherein R is selected from the group consisting of alkyl (C₁-C₆), halogen, —CN, COR₅COOR₅, N(R₆,R₇), CON (R₆, R₇), CH₂NO₂, NO₂, CH₂R₈, CHR₉, —CH═N—OR₁₀, —═CH—R₅, wherein R₅ is selected from the group consisting of H, optionally substituted C₁₋₁₂, alkyl, C₃₋₁₂, cycloalkyl, aryl, heteroaryl; R₆ and R₇ are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl, F, Cl, Br, C₁₋₁₂ alkyl substituted with one or more of F, Cl, Br, I, OR₄, SR₄, N(R₆,R₇) wherein R₄ is selected from the group consisting of H, C₁₋₁₂ alkyl, C₃₋₁₂ cycloalkyl, C₁₋₆,alkoxy, C₁₋₆ alkyl substituted with one or more F, Cl, Br, I or OH and R₆ and R₇ are the same as defined earlier, Rio is selected from the group consisting of H, optionally substituted C₁₋₁₂ alkyl, C₃₋₁₂ cycloalkyl, C₁₋₆, alkoxy, C₁₋₆ alkyl, aryl, heteroaryl;
n is an integer in the range from 0 to 3;
X is CH, CH—S, CH—O and N;
Y and Z are independently selected from the group consisting of hydrogen C₁₋₆ alkyl, C₃₋₁₂ cycloalkyl, C₀₋₃ bridging group;
U and V are independently selected from the group consisting of optionally substituted C₁₋₆ alkyl , F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁₁)CH₂—, —CO—CO—, CH₂ (R₁₁)N—, CH (R₁₁), S, CH₂(CO), N(R₁₁) wherein R₁₁ is optionally substituted with C₁₋₁₂ alkyl, C₃₋₁₂ cycloalkyl, C₁₋₆ alkoxy, C₁₋₆ alkyl , aryl , heteroaryl; and
R₁ is selected from the group consisting of —NHC(═O)R₂ wherein R₂ is hydrogen, C₁₋₁₂ alkyl, C₃₋₁₂ cycloalkyl, C₁₋₆ alkoxy, C₁-₆ alkyl substituted with one or more of F, Cl, Br, I or OH; N(R₃, R₄) ; —NR₂C(═S)R₃: —NR₂C(═S)SR₃ wherein R₂ is the same as defined above and R₃ and R₄ are independently selected from the group consisting of H, C₁₋₁₂ alkyl, C₃₋₁₂ cycloalkyl, C₁₋₆ alkoxy, C₁₋₆ alkyl substituted with one or more of F, Cl, Br, I or OH.

15. A method of treating or preventing catheter infections and foreign body or prosthesis infections in a mammal comprising administering to said mammal, a therapeutically effective amount of a compound having the structure of Formula II 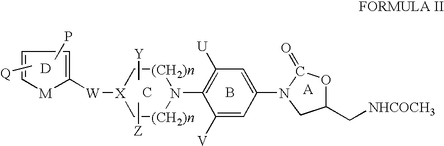
or its pharmaceutically acceptable salts, enantiomers, diastearomers, N-oxides, prodrugs or metabolites wherein
M=O, S, NH or N—CH₃;
X is CH, CH—S, CH—O and N;
Y and Z are independently selected from the group consisting of hydrogen, C₁₋₆ alkyl, C₃₋₁₂ cycloalkyl, C₀₋₃ bridging group;
U and V are independently selected from the group consisting of optionally substituted C₁₋₆ alkyl, F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁)CH₂—, —CO—CO—, CH₂ (R₁₁)N—, CH (R₁₁) , S, CH₂(CO), N(R₁₁) wherein R₁₁ is hydrogen, optionally substituted with C₁₋₁₂ alkyl, C₃₋₁₂ cycloalkyl, C₁₋₆ alkoxy, C₁₋₆ alkyl, aryl, heteroaryl except when M=S, Q=P=H, W=(C═O);
n is an integer in the range from 0 to 3; and,
Q and P are independently selected from the group consisting of hydrogen, —CN, COR₅, COOR₅, N (R₆, R₇), CON (R₆,R₇), CH₂NO₂, NO₂, CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, heteroaryl; R₆ and R₇ are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl ,F, Cl, Br, C₁₋₁₂ alkyl substituted with one or more of F, Cl, Br, I, OR₄, SR₄, wherein R₄ is selected from the group consisting of H, C₁₋₁₂ alkyl, C₃₋₁₂ cycloalkyl, C₁₋₆ alkoxy, C₁₋₆ alkyl substituted with one or more F, Cl, Br, I or OH, N(R₆, R₇), R₁₀ is selected from the group consisting of H, optionally substituted C₁₋₁₂ alkyl, C₃₋₁₂ cycloalkyl, C₁₋₆ alkoxy, C₁₋₆ alkyl , aryl , heteroaryl except W=(CO), Q, P=H and M=S ring C in Formula II is 6-8 membered or of larger size and the larger rings have either two or three carbons between each nitrogen atom, comprising of 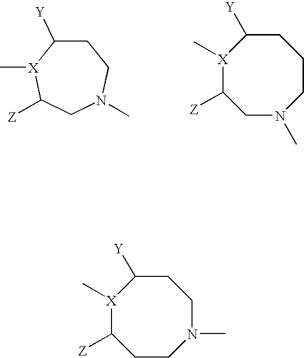
and may be bridged to form a bicyclic system as shown below, 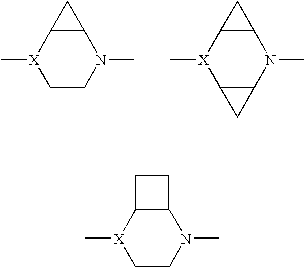
ring C is optionally substituted by Y and Z with alkyl groups, cycloalkyl groups, fluoro group, carboxylic and corresponding esters, amides, substituted alkyls or bridging alkyl groups are a shown below: 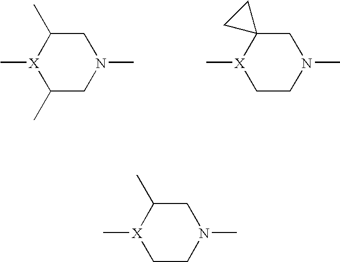
six membered ring C with X=—CH—(NR₁₁), (wherein R₁₁ is the same as defined earlier) is selected from the group consisting of the following rings;

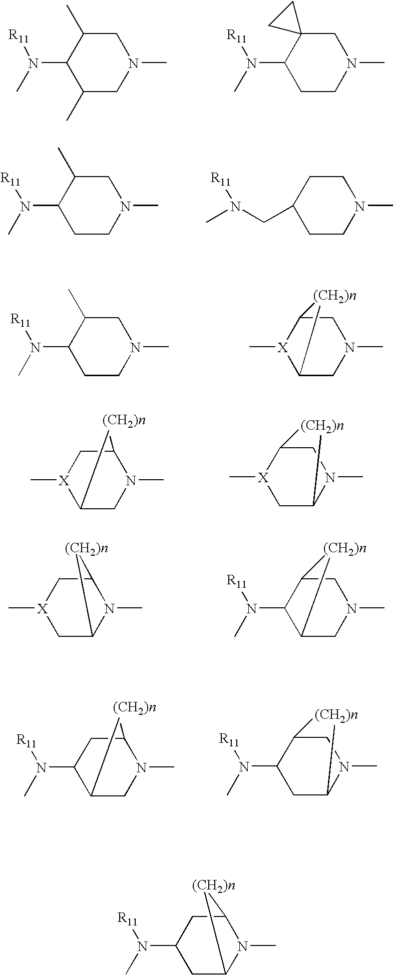

16. A method of treating or preventing catheter infections and foreign body or prosthesis infections in a mammal comprising administering to said mammal, a therapeutically effective amount of a compound namely, (S)-N-[[3-Fluoro-4-[N-1[4-{ 2-furyl(5-nitro)methyl }]piperazinyl]phenyl]-2-oxo-5-oxazolidinyl]methyl]acetamide hydrochloride.

17. A process for preparing a compound of Formula I 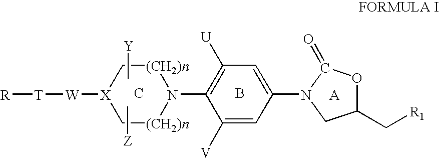
and its pharmaceutically acceptable salts, enantiomers, diastearomers, N-oxides, prodrugs or metabolites, wherein
T is five to seven membered heterocyclic ring, aryl, substituted aryl, bound to the ring C with a linker W and the heterocyclic and aryl rings are further substituted by a group represented by R,
wherein R is selected from the group consisting of alkyl (C₁-C₆), halogen, —CN, COR₅,COOR₅, N(R₆,R₇), CON (R₆, R₇), CH₂NO₂, NO₂, CH₂R₈, CHR₉, —CH═N—OR₁₀, —C═CH—R₅, wherein R₅ is selected from the group consisting of H, optionally substituted C₁-C₁₂, alkyl, C₃₋₁₂, cycloalkyl, aryl, heteroaryl, R₆ and R₇, are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl, F, Cl, Br, C₁₋₁₂ alkyl substituted with one or more of F, Cl, Br, I, OR₄, SR₄, N(R₆,R₇) wherein R₄ is selected from the group consisting of H, C₁₋₁₂ alkyl, C₃₋₁₂ cycloalkyl, C₁₋₆ alkoxy, C₁₋₆alkyl substituted with one or more F, Cl, Br, I or OH and R₆ and R₇ are the same as defined earlier, R₁₀ is selected from the group consisting of H, optionally substituted C₁₋₁₂ alkyl, C₃₋₁₂ cycloalkyl, C₁₋₆, alkoxy, C₁₋₆ alkyl, aryl, heteroaryl;
n is an integer in the range from 0 to 3;
X is CH, CH—S, CH—O and N;
Y and Z are independently selected from the group consisting of hydrogen, C₁₋₆ alkyl, C₃₋₁₂ cycloalkyl, C₀₋₃ bridging group;
U and V are independently selected from the group consisting of optionally substituted C₁₋₆ alkyl, F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁₁)CH₂—, —CO—CO—, CH₂ (R₁₁)N—, CH (R₁₁), S, CH₂(CO), N(R₁₁) wherein R₁₁ is optionally substituted with C₁₋₁₂ alkyl, C₃₋₁₂ cycloalkyl, C₁₋₆ alkoxy, C₁₋₆ alkyl, aryl, heteroaryl; and
R₁ is selected from the group consisting of —NHC(═O)R₂ wherein R₂ is hydrogen, C₁₋₁₂ alkyl, C₃₋₁₂ cycloalkyl, C₁₋₆ alkoxy, C₁₋₆ alkyl substituted with one or more of F, Cl, Br, I or OH; N(R₃, R₄) ; —NR₂C(═S) R₃: —NR₂C(═S)SR₃ wherein R₂ is the same as defined above and R₃ and R₄ are independently selected from the group consisting of H, C₁₋₁₂ alkyl, C₃₋₁₂ cycloalkyl, C₁₋₆ alkoxy, C₁₋₆ alkyl substituted with one or more of F, Cl, Br, I or OH, which comprises reacting an amine compound of Formula V 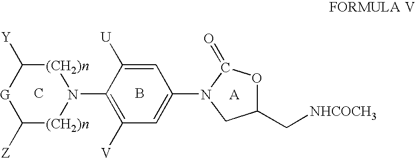
with a heterocyclic compound of Formula R-T-W-R₁₂ wherein G in amines of Formula V is defined as NH, CH(NHR₁₃), —CH—CH₂NHR₁₃ wherein R₁₃ is H, ethyl, methyl, isopropyl,acetyl, cyclopropyl, alkoxy or acetyl and Y, Z, U, V, R₁, n, R, T and W are the same as defined earlier and R₁₂ is a suitable leaving group selected from the group comprising of fluoro, chloro, bromo, SCH₃, —SO₂CH₃, —SO₂CF₃ or OC₆H₅.

18. A process for preparing a compound of Formula I as claimed in claim 17, wherein W=CH₂ and R-T-W—R₁₂ is a five membered heterocyclic ring with aldehyde group and the compound of Formula I is produced by reductive amination.

19. A process for preparing a compound of Formula I as claimed in claim 17, wherein W=CO and R-T-W-R₁₂ is a five membered heterocyclic ring with carboxylic acid, and amino compound of Formula V is acylated with activated esters in presence of condensing agents comprising 1,3-dicyclohexylcarbodiimide (DCC) and 1-(3-dimethylaminopropyl)-3-ethylcarbodiimide (EDC).

20. A process for the preparation of compound of Formula II 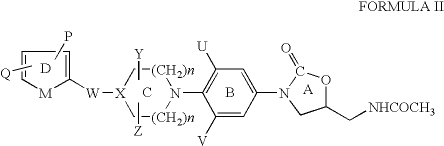
wherein
M=O, S, NH and N—CH₃;
n is an integer in the range from 0 to 3;
X is CH, CH—S, CH—O and N;
Y and Z are independently selected from the group consisting of hydrogen, C₁₋₆ alkyl, C₃₋₁₂ cycloalkyl, C₀₋₃ bridging group;
U and V are independently selected from the group consisting of optionally substituted C₁₋₆ alkyl, F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁₁)CH₂—, —CO—CO—, CH₂ (R₁₁)N—, CH (R₁₁), S, CH₂(CO), N(R₁₁) wherein R₁₁ is hydrogen, optionally substituted with C₁₋₁₂ alkyl, C₃₋₁₂ cycloalkyl, C₁₋₆ alkoxy, C₁₋₆ alkyl, aryl, heteroaryl; and
Q and P are independently selected from the group consisting of —CN, COR₅, COOR₅, N (R₆, R₇), CON (R₆,R₇), CH₂NO₂, NO₂, CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, heteroaryl; R₆ and R₇ are independently selected from the group consisting of H, optionally substituted C₁₋₁₂ alkyl, C₃₋₁₂ cycloalkyl, C₁₋₆ alkoxy; R₈ and R₉ are independently selected from the group consisting of H, C₁₋₆ alkyl, F, Cl, Br, C₁₋₁₂ alkyl substituted with one or more of F, Cl, Br, I, OR₄, SR₄, wherein R₄ is the same as defined before, N(R₆, R₇), R₁₀ is selected from the group consisting of H, optionally substituted C₁₋₁₂ alkyl, C₃₋₁₂ cycloalkyl, C₁₋₆ alkoxy, C₁₋₆ alkyl, aryl, heteroaryl except when M=S, W=(CO), Q and P=H.
Ring C in Formula II is 6-8 membered or of larger size and the larger rings have either two or three carbons between each nitrogen atom, comprising of 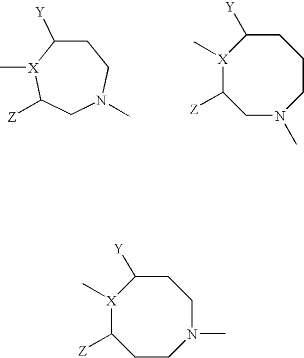
and may be bridged to form a bicyclic system as shown below, 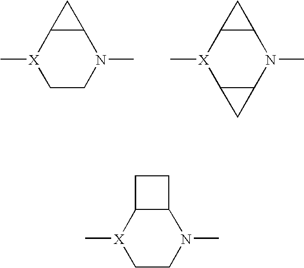
ring C is optionally substituted by Y and Z with alkyl groups, cycloalkyl groups, fluoro group, carboxylic and corresponding esters, amides, substituted alkyls or bridging alkyl groups are as shown below: 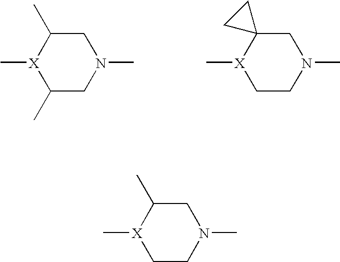
six membered ring C with X=—CH—(NR₁₁), (wherein R₁₁ is the same as defined earlier) is selected from the group consisting of the following rings; 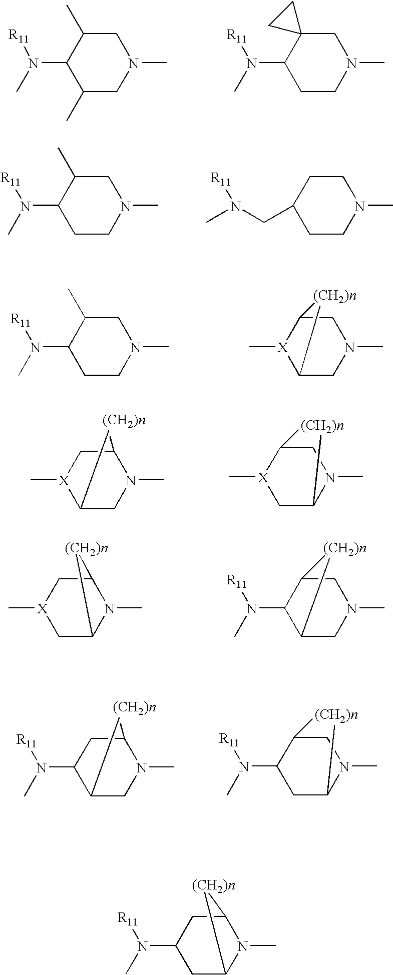
wherein M=Sulphur is shown by compounds of Formula III, 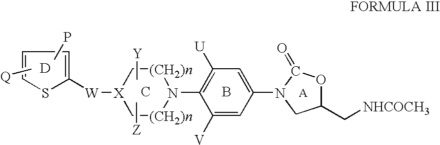
wherein P, Q, U, V, X, Y, Z, W and n in Formula III are the same as previously defined, wherein the process comprising reacting a compound of Formula V 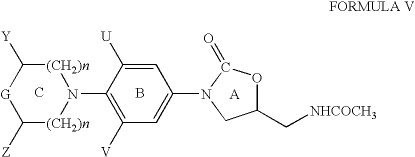
with a compound of Formula VI 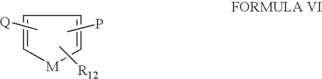
wherein M, P, Q, R₁₂, Y, Z, G, n, U and V are the same as defined earlier.

21. A process for preparing a compound of Formula II as claimed in claim 20, in a suitable solvent selected from the group consisting of dimethylformamide, dimethylacetamide, ethanol or ethylene glycol at a suitable temperature in the range of —70° C. to 180° C. in the presence of a suitable base selected from the group consisting of triethyl amine, diisopropyl amine, potassium carbonate and sodium bicarbonate.

22. A process of preparing a compound of Formula II as claimed in claim 20 wherein Formula VI is furaldehyde and reductive alkylation of the amine of Formula V is performed with a reducing agent.

23. A process for preparing a compound of Formula II as claimed in claim 20 wherein Formula VI is furoic acid.

24. A process for preparing a compound of Formula II as claimed in claim 20 wherein the compounds of Formula II having carbonyl link are prepared by reacting heteroaromatic compound of the Formula VI including N-methyl pyrrole with the intermediate amine of Formula V in the presence of triphosgene or phosgene and carbonyl linkers are introduced between heteroaromatic compound comprising reacting 3-bromothiophene and amine of Formula V with carbon monoxide and the catalyst is selected from the group consisting of Pd(PPh₃)₂Cl₂ and extended chain pyrroles having dicarbonyl linkers are obtained by treatment of oxalyl chloride and amine of the Formula V.

25. A process for preparing a compound of Formula VIII 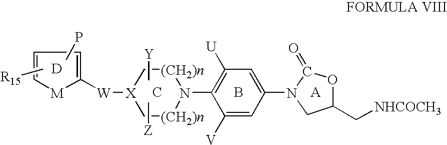
wherein
M=O, S, NH and NCH₃;
n is an integer in the range from 0 to 3;
X is CH, CH—S, CH—O and N;
Y and Z are independently selected from the group consisting of hydrogen, C₁₋₆ alkyl, C₃₋₁₂ cycloalkyl, C₀₋₃ bridging group;
U and V are independently selected from the group consisting of optionally substituted C₁₋₆ alkyl, F, Cl, Br, C₁₋₁₂ alkyl substituted with one or more of F, Cl, Br, I, preferably U and V are hydrogen or fluoro;
W is selected from the group consisting of CH₂, CO, CH₂NH, —NHCH₂, —CH₂NHCH₂, —CH₂—N (R₁₁)CH₂—, —CO—CO—, CH₂(R₁₁)N—, CH(R₁₁), S, CH₂(CO), N(R₁₁) wherein R₁₁ is hydrogen, optionally substituted with C₁₋₁₂ alkyl, C₃₋₁₂ cycloalkyl, C₁₋₆ alkoxy, C₁₋₆ alkyl, aryl, heteroaryl;
Q and P are independently selected from the group consisting of (C₁-C₆) alkyl halogen, —CN, COR₅, COOR₅, N (R₆, R₇) CON(R₆,R₇), CH₂NO₂, NO₂, CH₂R₈, CHR₉, —CH═N—OR₁₀, C═CH—R₅, wherein R₅ is selected from the group consisting of H, optionally substituted C₁₋₁₂alkyl, C₃₋₁₂ cycloalkyl, aryl, heteroaryl; R₆ and R₇ are independently selected from the group consisting of H, optionally substituted C₁₋₁₂ alkyl, C₃₋₁₂ cycloalkyl, C ₁₋₆ alkoxy; R₈ and R₉ are independently selected from the group consisting of H, C₁₋₆ alkyl, F, Cl, Br, C₁₋₁₂ alkyl substituted with one or more of F, Cl, Br, I, OR₄, SR₄,wherein R₄ is the same as defined before, N(R₆, R₇), R₁₀ is selected from the group consisting of H, optionally substituted C₁₋₁₂ alkyl, C₃₋₁₂ cycloalkyl, C₁₋₆ alkoxy, C₁₋₆ alkyl, aryl, heteroaryl except when W=(CO), Q and P=H and M=S.
M=Sulphur is shown by compounds of Formula III 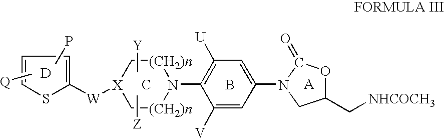
and R₁₅ is the same as Q defined earlier, comprising converting a compound of Formula VII 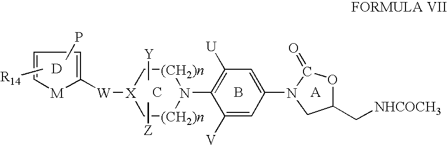
wherein in U, V, Y, Z, X, W, P, n and M are the same as defined earlier and are R₁₄ is any group which can be converted to group R₁₅ in one to five steps.

26. A process for preparing a compound of Formula XI 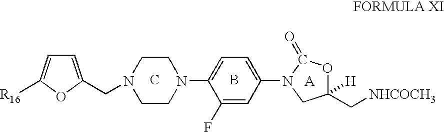
(R₁₆=—CH₂F or —CH₂F₂) by reacting a compound of Formula IX 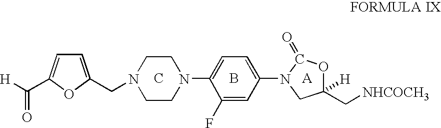
with sodium borohydride to produce a compound of Formula X 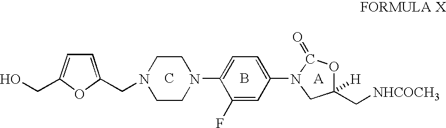
and further reacting this compound with diethylamino sulfurtrifluoride to produce compound of Formula XI.

27. A process for preparing a compound of Formula XII 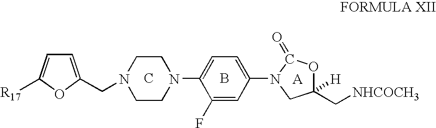
wherein R₁₇= 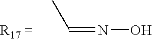
which comprises reacting (S)-N-[[3-Fluoro-4-[N-1[4-{2-furyl(5-formyl)methyl }]piperazinyl]phenyl]-2-oxo-5-oxazolidinyl]-methyl]acetamide of Formula IX 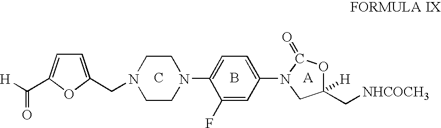
with hydroxylamine.

28. A process for preparing a compound of Formula XII 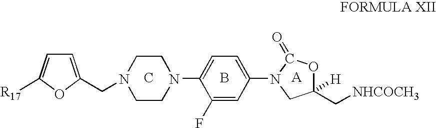
wherein R₁₇= 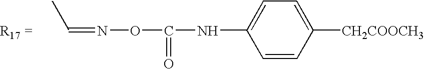
which comprises reacting (S)-N-[[3-[3-Fluoro-4[N-1-[4-{2-furyl-(5-hydrazone)-methyl}]-piperazinyl]-phenyl]-2-oxo-5-oxazolidinyl]-methyl]acetamide with hydrazine hydrate.

29. A process for preparing a compound of Formula XII 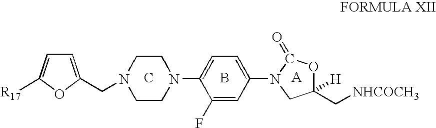
wherein R₁₇= 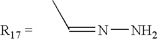
which comprises reacting (S)-N-[[3-[3-Fluoro-4-[N-1-[4-(2-furyl-(5-aldoxime)methyl)]piperazinyl]phenyl]-2-oxo-5-oxazolidinyl]methyl]acetamide with isocyanate.

30. A process for preparing a compound of Formula XII 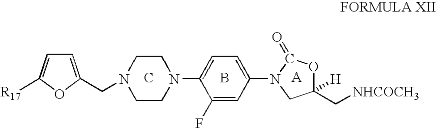
wherein R₁₇=CN which comprises reacting (S)-N-[[3-[3-Fluoro-4-[N-1[4-[2-furyl(5-cyano)methyl}]piperazinyl]phenyl]-2-oxo-5-oxazolidinyl]methyl]acetamide with trifilic anhydride and triethylamine.

31. A process for preparing a compound of Formula XII 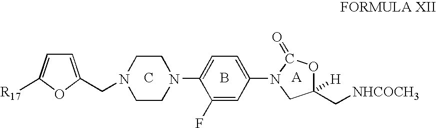
wherein 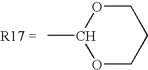
which comprises reacting (S)-N-[[3-Fluoro-4-[N-1[5-(1,3-dioxane)-2-furylmethyl]piperazinyl]phenyl]-2-oxo-5-oxazolidinyl]methyl]acetamide with 1,3-propane diol and BF₃ etherate.

32. A process for the preparation of the compound of Formula XIV 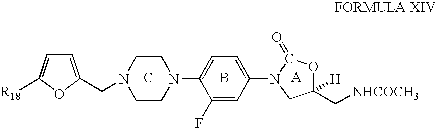
wherein 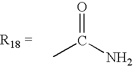
which comprises reacting (S)-N-[[3-Fluoro-4-[N-1[4-{2-furyl(5-formyl)methyl}]piperazinyl]phenyl]-2-oxo-5-oxazolidinyl]methyl]acetamide of Formula IX 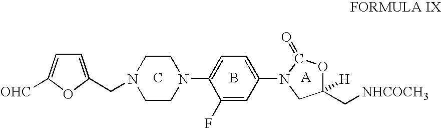
with Ag₂O to produce (S)-N-[[3-Fluoro-4-[N-1[4-{2-furyl(5-carboxy)methyl}]piperazinyl]phenyl]-2-oxo-5-oxazolidinyl]methyl]acetamide of Formula XIII followed by reacting (S)-N-[[3-Fluoro-4-[N-1[4-(2-furyl-(5-carboxy-ethyl)methyl)piperazinyl]phenyl]-2-oxo-5-oxazolidinyl]methyl]acetamide of Formula XIII 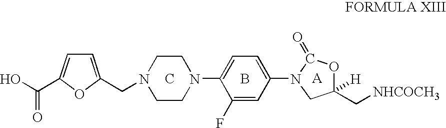
with aqueous ammonia to produce Formula XIV.

33. A process for the preparation of the compound of Formula XIV 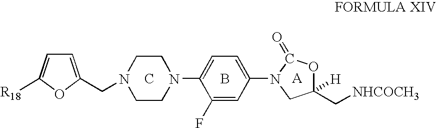
wherein 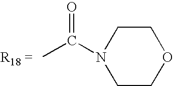
which comprises reacting (S)-N-[[3-Fluoro-4-[N-1[4-{2-furyl(5-formyl)methyl}]piperazinyl]phenyl]-2-oxo-5-oxazolidinyl]methyl]acetamide of Formula IX 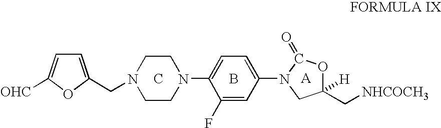
with Ag₂O to produce (S)-N-[[3-Fluoro-4-[N-1[4-{2-furyl(5-carboxy)methyl}]piperazinyl]phenyl]-2-oxo-5-oxazolidinyl]methyl]acetamide of Formula XIII followed by reacting (S)-N-[[3-Fluoro-4-[N-1[4-(2-furyl-(5-carboxy-ethyl)methyl)piperazinyl]phenyl]-2-oxo-5-oxazolidinyl]methyl]acetamide of Formula XIII 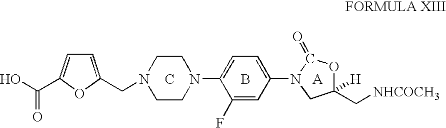
with thionyl chloride to produce Formula XIV.

34. A process for the preparation of the compound of Formula XIV 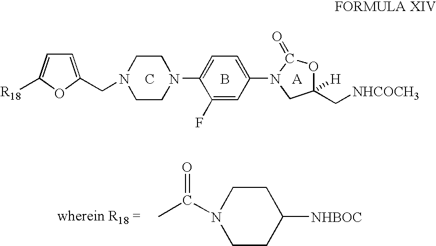
which comprises reacting (S)-N-[[3-Fluoro-4-[N-1[4-{2-furyl(5-formyl)methyl}]piperazinyl]phenyl]-2-oxo-5-oxazolidinyl]methyl]acetamide of Formula IX 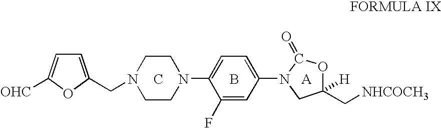
with Ag₂O to produce (S)-N-[[3-Fluoro-4-[N-1[4-(2-furyl(5-carboxy)meth-yl-3]piperazinyl]phenyl]-2-oxo-5-oxazolidinyl]methyl] acetamide of Formula XIII followed by reacting (S)-N-[[3-Fluoro-4-[N-1[4-(2-furyl-(5-carboxy-ethyl)methyl)piperazinyl]phenyl]-2-oxo-5-oxazolidinyl]methyl]acetamide of Formula XIII 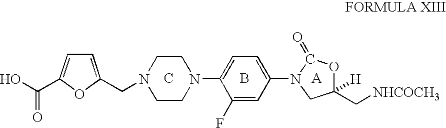
with morpholine in the presence of oxalyl chloride to produce Formula XIV.
